(19) United States
(12) Patent Application Publication
(10) Pub. No.: US 2016/0002273 A1
(43) Pub. Date: Jan. 7, 2016

(54) THIENO[3,2-D]PYRIMIDINE-6-CARBOXAMIDES AND ANALOGUES AS SIRTUIN MODULATORS

(71) Applicant: GLAXOSMITHKLINE LLC, Wilmington,, DE (US)

(72) Inventors: Charles A. BLUM, Waltham, MA (US); Jeremy S. DISCH, Natick, MA (US); Ghotas EVINDAR, Waltham, MA (US); Robert B. PERNI, Marlborough, MA (US)

(21) Appl. No.: 14/771,334

(22) PCT Filed: Mar. 7, 2014
(86) PCT No.: PCT/US14/21683

§ 371 Date: Aug. 28, 2015

Related U.S. Application Data
(60) Provisional application No. 61/774,101, filed on Mar. 7, 2013.

(51) Int. Cl. C07F 9/6561; C12Q 1/34; C07D 519/00; C07D 495/04; C07D 491/048

(52) U.S. Cl. CPC C07F 9/65616; C07D 495/04; C07D 491/048; C07D 519/00; C12Q 1/34

(57) ABSTRACT

Provided herein are novel substituted thieno[3,2-d]pyrimidine-6-carboxamide sirtuin inhibitors and methods of use thereof. The sirtuin inhibitors may be used for inhibiting a sirtuin-mediated biological process, and, e.g. for treating and/or preventing diseases and disorders including, but not limited to cancer, neurodegenerative disease and inflammation. Also provided herein are pharmaceutical compositions comprising these sirtuin inhibitors and compositions comprising a sirtuin inhibitor in combination with another therapeutic agent.

### BACKGROUND

The Silent Information Regulator (SIR) family of genes represents a highly conserved group of genes present in the genomes of organisms ranging from archaebacteria to eukaryotes. The encoded SIR proteins are involved in diverse processes from regulation of gene silencing to DNA repair. A well-characterized gene in this family is S. cerevisiae SIR2, which is involved in silencing HM loci that contain information specifying yeast mating type, telomere position effects and cell aging. The yeast Sir2 protein belongs to a family of histone deacetylases. The proteins encoded by members of the SIR gene family show high sequence conservation in a 250 amino acid core domain. The Sir2 homolog, CobB, in Salmonella typhimurium, functions as an NAD (nicotinamide adenine dinucleotide)-dependent ADP-ribosyl transferase.

The Sir2 protein is a class III deacetylase which uses NAD as a cosubstrate. Unlike other deacetylases, many of which are involved in gene silencing, Sir2 is insensitive to class I and II histone deacetylase inhibitors like trichostatin A (TSA).

Deacetylation of acetyl-lysine by Sir2 is tightly coupled to NAD hydrolysis, producing nicotinamide and a novel acetyl-ADP ribose compound (i.e., 2′/3′-O-acetyl-ADP-ribose (OAADPR)). The NAD-dependent deacetylase activity of Sir2 is essential for its functions, which can connect its biological role with cellular metabolism in yeast. Mammalian Sir2 homologs have NAD-dependent histone deacetylase activity.

Biochemical studies have shown that Sir2 can readily deacetylate the amino-terminal tails of histones H3 and H4, resulting in the formation of OAADPR and nicotinamide. Strains with additional copies of SIR2 display increased rDNA silencing and a 30% longer life span. It has also been shown that additional copies of the C. elegans SIR2 homolog (sir-2.1) and the D. melanogaster (dSir2) gene extend life span in those organisms. This implies that the SIR2-dependent regulatory pathway for aging arose early in evolution and has been conserved throughout eukaryotic evolution. Today, Sir2 genes are believed to have evolved to enhance an organism's health and stress resistance to increase its chance of surviving adversity.

In humans, there are seven Sir2-like genes (SIRT1-SIRT7) that share the conserved catalytic domain of Sir2. SIRT1 is a nuclear protein with the highest degree of sequence similarity to Sir2. SIRT1 regulates multiple cellular targets by deacetylation including the tumor suppressor p53, the cellular signaling factor NF-κB, and the FOXO transcription factor.

SIRT2 and SIRT3 are homologs of SIRT1, and possess NAD⁺-dependent protein deacetylase activity (Baur et al. 2012 Nature Reviews, 11, 443-461). In addition, SIRT 2 and 3 are ubiquitously expressed (Botta et al. 2012 Curr. Med. Chem, 19, 5871-5884.). SIRT2 is a tubulin deacetylase located predominately a cytoplasmic protein, where it regulates normal mitotic progression (Botta et al. 2012 Curr. Med. Chem, 19, 5871-5884). The SIRT3 protein is targeted to the mitochondrial cristae by a unique domain located at the N-terminus, and is ubiquitously expressed, particularly in metabolically active tissues. Upon transfer to the mitochondria, SIRT3 is believed to be cleaved into a smaller, active form by a mitochondrial matrix processing peptidase (MPP) (Shi et al. 2005 JBC, 14, 13560-13567).

Modulation of sirtuin activity, either through activation or inhibition has been reported to be beneficial in numerous disease states including metabolic (Banks, A. S. et al. (2008) Cell Metab 8, 333-341), cancer (Peck, B. et al. (2010) Mol Cancer Ther 9, 844-855 and Wang et al. (2008) Cancer Cell 14, 312-323), neurodegeneration (Liu, L. et al. (2012) J Biol Chem 287, 32307-32311; Tang, B. L. et al. (2009) Cell Mol Neurobiol 29, 1093-1103 and Outeiro, T. F. et al. (2007) Science 317, 516-519), inflammation (Yoshizaki, T. et al. (2009) Mol Cell Biol 29, 1363-1374 and Yoshizaki, T. et al. (2010) Am J Physiol Endocrinol Metab 298, E419-E428) and ischaemic injury (Narayan, N. et al. (2012) Nature 492, 199-204.

Recently, it has been reported that the function of these enzymes is dependent on their cellular localization and type of tissue where the cells reside (Bauer, J. A. et al. (2012) Nat Rev Drug Disc 11, 443-461), however our understanding of sirtuin function is far from complete.

There is evidence that the pharmacological modulation of sirtuin function could find significant clinical applications. For example, simultaneous inhibition of SIRT1 and SIRT2 may be beneficial against cancers by inhibiting the sirtuin mediated deacetylation of p53 leading to cell death, though inhibiting SIRT1 or SIRT2 individually was insufficient for inhibition of the deacetylation of p53 in vivo (Peck, B. et al. (2010) Mol Cancer Ther 9, 844-855). In a neurodegenerative setting, evidence suggests that SIRT2 mediated deacetylation promotes neuronal damage via FOXO3a deacetylation, and it was demonstrated that the genetic deletion of SIRT2 leads to a reduction of apotosis in mice (Liu, L. et al. (2012) J Biol Chem 287, 32307-32311). A recent review reports that SIRT3 may play a role in reglating central pathways of mitochondrial metabolism and mitochondrial respiration (Botta et al. (2012) Curr Med Chem 19, 5871-5884).

Due to the largerly conserved catalytic core of SIRT1-SIRT7, one area of interest is the inhibition of multiple sirtuin isoforms, specifically SIRT1, SIRT2 and SIRT3.

To date, there have been several reports identifying sirtuin inhibitors, primarily SIRT1 and SIRT2 inhibitors. Among the earliest SIRT1/SIRT2 inhibitors identified are sirtinol (Bauer, J. A. et al. (2012) Nat Rev Drug Disc 11, 443-461), and the closely related salermide (Finkel, T. et al. (2009) Nature 460, 587-591). Suramin (Banks, A. S. et al. (2008) Cell Metab 8, 333-341), inhibits both SIRT1 and SIRT2, but exhibits poor selectivity (Trapp, J. et al. (2007) Chem Med Chem 2, 1419-1431), whereas EX-527 (Peck, B. et al. (2010) 9, 844-855) exhibits a high degree of selectivity for SIRT1 over SIRT2 and SIRT3 (Napper, A. D. et al. (2005) 48, 8045-8054). EX-527 is among the most studied of the published inhibitors and has been used as both a standard inhibitor in biological studies and as a screening tool for identifying novel inhibitor scaffolds. To date, a broad spectrum of compound classes have demonstrated sirtuin inhibition (Sanders, B. D. et al. (2009) Bioorg Med Chem 17, 7031-7041) ranging from peptide substrate mimetics (Kiviranta, P. H. et al. (2009) J Med Chem 52, 2153-2156 and Tervo, A. J. et al. (2006) J Med Chem 49, 7239-7241) to heterocyclic small molecules such as cambinol (Heltweg, B. et al. (2006) Cancer Res 66, 4368-4377). These inhibitors generally exhibit micromolar to high nanomolar IC₅₀ values and are moderately SIRT1 selective, except for the equipotent SIRT1/SIRT2 inhibitor Cambinol.

Recently, a number of novel selective SIRT2 inhibitors have been reported. For example, Suzuki, T. et al. (2012 J Med Chem 55, 5760-5773) reported a selective 2-anilinobenzamide inhibitor that exhibits >500:1 preference for SIRT2 over SIRT1 and SIRT3 and Friden-Saxin, M. et al. (2012 J Med Chem 55, 7104-7113) disclosed a selective chromenone inhibitor that shows a >500:1 preference for SIRT2 over SIRT1 and SIRT3 and exhibits less than 10% inhibition at 200 mM against SIRT1/3. In addition, Galli, et al. ((2012) Eur J Med Chem 55, 58-66) reported 3-(1H-1,2,3-triazol-4-yl)pyridine, a nicotinamide analogue, that exhibited modest selective for SIRT3 (IC₅₀=38 μM) over SIRT1 and SIRT2 (16%, 88% and 92% activity remaining at 1 mM respectively) and it demonstrated modest antiproliferative effects against several cancer cell lines. In general these inhibitors exhibit micromolar or high nanomolar potencies and tend to be at least moderately SIRT1 selective.

In addition to therapeutic potential, new and potent sirtuin inhibitors would be useful to advance understanding of the biological function of sirtuins, to further the understanding of the mechanism of action of sirtuin inhibition and to aid in the development of assays that identify novel sirtuin modulators.

### SUMMARY

One aspect of the present invention relates to novel thieno[3,2-d]pyrimidine-6-carboxamide analogues, including compounds of Structural Formulas (I) (e.g., Ia, Ib, and Ic), as are described in detail below. A second aspect of the present invention relates to the use of the novel thieno[3,2-d]pyrimidine-6-carboxamide analogues as sirtuin modulators, or compositions comprising sirtuin-modulating compounds. A third aspect of the invention relates to the use of the novel thieno[3,2-d]pyrimidine-6-carboxamide analogues as sirtuin inhibitors, or compositions comprising sirtuin inhibitors. A fourth aspect of the present invention relates to the use of the novel thieno[3,2-d]pyrimidine-6-carboxamide analogues as inhibitors of SIRT1, SIRT2 and SIRT3, or compositions comprising inhibitors of SIRT1, SIRT2 and SIRT3. Another aspect of the present invention provides methods for using compounds of the present invention, or compositions comprising compounds of the present invention, for treating numerous mammalian disorders and diseases.

In certain embodiments, compounds of the present invention, or compositions comprising compounds of the present invention that decrease the level and/or activity of a sirtuin protein may be used for numerous therapeutic applications, including but not limited to treating and/or preventing diseases related to metabolic diseases, inflammation, treatment of cancer, neurodegenerative diseases, ischaemic injury, or complications thereof, etc.

As described further below, the methods comprise administering to a mammalian subject in need thereof a pharmaceutically effective amount of a compound of the present invention, or compositions compounds of the present invention.

In certain aspects, the compounds of the present invention may be administered alone or in combination with other compounds, including other sirtuin-modulating compounds, or other therapeutic agents.

### BRIEF DESCRIPTION OF THE FIGURES

FIG. 1 depicts the chemical structures of sirtuin inhibitors reported in the literature.

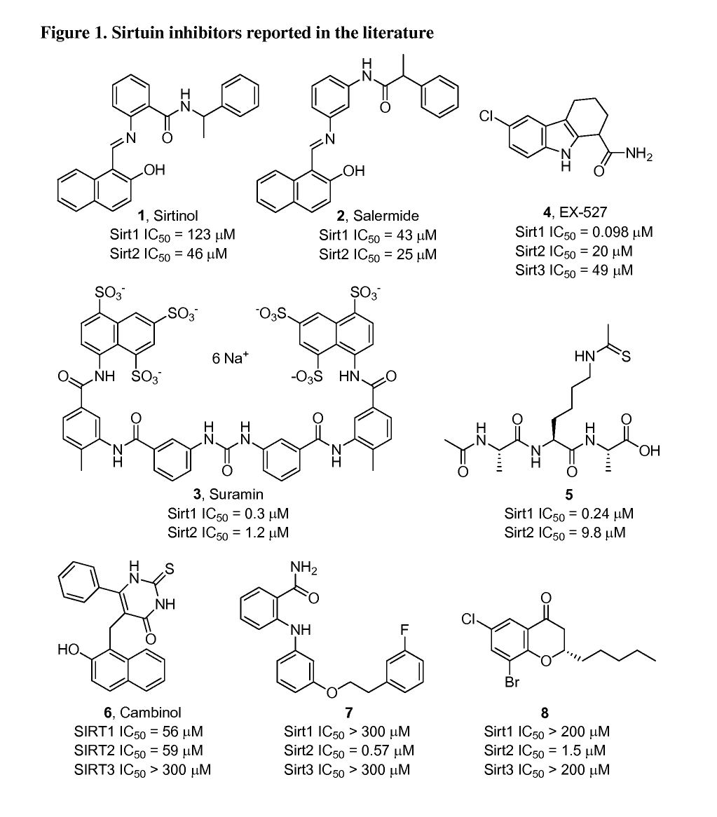

FIG. 2 shows a generalized structure of thieno[3,2-d]pyrimidin-6-carboxamide SIRT1/2/3 inhibitor.

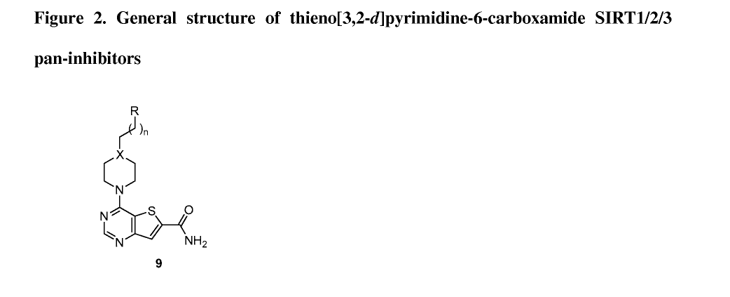

FIG. 3 depicts the general structure for the 3-cycle linear ELT screening library.

FIG. 4 shows the Spotfire™ cube data analysis from the SIRT3 ELT affinity screen.

FIG. 5 shows the synthetic scheme for the preparation of Compounds 11a, 11b, 11c and 11d.

FIG. 6 shows sirtuin mediated deacetylation of acetyl-p65 with Compounds 25, 28 and EX-527.

### DETAILED DESCRIPTION

### 1. Definitions

As used herein, the following terms and phrases shall have the meanings set forth below. Unless defined otherwise, all technical and scientific terms used herein have the same meaning as commonly understood to one of ordinary skill in the art.

The term “ED₅₀” refers to the art-recognized measure of effective dose. In certain embodiments, ED₅₀ means the dose of a drug which produces 50% of its maximum response or effect, or alternatively, the dose which produces a pre-determined response in 50% of test subjects or preparations, such as isolated tissue or cells. The term “LD₅₀” refers to the art-recognized measure of lethal dose. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The term “IC₅₀” is art-recognized and refers to the dose of a drug which produces 50% of its maximum response or effect. In other words, it is the half maximal inhibitory concentration of a drug.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues.

The term “bioavailable”, when referring to a compound, is art-recognized and refers to a form of a compound that allows for all or a portion of the amount of compound administered to be absorbed by, incorporated into, or otherwise physiologically available to a subject or patient to whom it is administered.

“Biologically active portion of a sirtuin” refers to a portion of a sirtuin protein having a biological activity, such as the ability to deacetylate (“catalytically active”). Catalytically active portions of a sirtuin may contain, but are not limited to, the core domain of sirtuins. Catalytically active portions of SIRT1 having GenBank Accession No. NP_{—}036370 that encompass the NAD⁺ binding domain and the substrate binding domain, for example, may include without limitation, amino acids 240-664 or 240-505 of GenBank Accession No. NP_{—}036370, which are encoded by the polynucleotide of GenBank Accession No. NM_{—}012238. Therefore, this region is sometimes referred to as the core domain. Other catalytically active portions of SIRT1, also sometimes referred to as core domains, include about amino acids 242 to 493 of GenBank Accession No. NP_{—}036370, which are encoded by nucleotides 777 to 1532 of GenBank Accession No. NM_{—}012238, or about amino acids 240 to 505 of GenBank Accession No. NP_{—}036370, which are encoded by the polynucleotide of GenBank Accession No. NM_{—}012238. Another “biologically active” portion of SIRT1 is amino acids 183-225 of GenBank Accession No. NP_{—}036370, which comprise a domain N-terminal to the core domain that is important to the compound binding site.

Catalytically active portions of SIRT2 having GenBank Accession No. NP_{—}036369.2 that encompass the NAD⁺ binding domain and the substrate binding domain, for example, may include without limitation, amino acids 57-356 of GenBank Accession No. NP_{—}036369.2, which are encoded by the polynucleotide of GenBank Accession No. NM_{—}012237.3. Therefore, this region is sometimes referred to as the core domain.

Catalytically active portions of SIRT3 having GenBank Accession No. NP_{—}036371.1 that encompass the NAD⁺ binding domain and the substrate binding domain, for example, may include without limitation, amino acids 118-399 of GenBank Accession No. NP_{—}036371.1, which are encoded by the polynucleotide of GenBank Accession No. NM_{—}012239.5. Therefore, this region is sometimes referred to as the core domain.

The term “mammal” is known in the art, and exemplary mammals include humans, primates, livestock animals (including bovines, porcines, etc.), companion animals (e.g., canines, felines, etc.) and rodents (e.g., mice and rats).

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal, and intrasternal injection and infusion.

A “patient”, “subject”, “individual” or “host” refers to either a human or a non-human animal.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

“Sirtuin-modulating compound” refers to a compound that is either a sirtuin inhibitor compound or a sirtuin activator compound.

“Sirtuin-activating compound” or “sirtuin activator compound” refers to a compound that increases the level of a sirtuin protein and/or increases at least one activity of a sirtuin protein. In an exemplary embodiment, a sirtuin-activating compound may increase at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; mitotic regulation and controlling the segregation of oxidized proteins between mother and daughter cells.

“Sirtuin-inhibiting compound” or “sirtuin inhibitor compound” refers to a compound that decreases the level of a sirtuin protein and/or decreases at least one activity of a sirtuin protein. In an exemplary embodiment, a sirtuin-inhibiting compound may decrease at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells.

“SIRT1/2/3 inhibitor” refers to a sirtuin inhibitor that decreases at least one biological activity of SIRT1, SIRT2, and SIRT3 proteins by at least about 10%, 25%, 50%, 75%, 100%, or more. Exemplary biological activities of SIRT1, SIRT2, and SIRT3 proteins include deacetylation, e.g., of an acetylated peptide substrate.

“Sirtuin pan-inhibitor” refers to a sirtuin inhibitor that decreases at least one biological activity of two or more sirtuin deacetylase proteins (e.g., SIRT1 and SIRT2) by at least about 10%, 25%, 50%, 75%, 100%, or more. Exemplary biological activities of sirtuin proteins include deacetylation, e.g., of an acetylated peptide substrate.

“Sirtuin protein” refers to a member of the sirtuin deacetylase protein family, or preferably to the sir2 family, which include yeast Sir2 (GenBank Accession No. P53685), C. elegans Sir-2.1 (GenBank Accession No. NP_{—}501912), and human SIRT1 (GenBank Accession No. NM_{—}012238 and NP_{—}036370 (or AF083106)) and SIRT2 (GenBank Accession No. NM_{—}012237, NM_{—}030593, NP_{—}036369, NP_{—}085096, and AF083107) proteins. Other family members include the four additional yeast Sir2-like genes termed “HST genes” (homologues of Sir two) HST1, HST2, HST3 and HST4, and the five other human homologues hSIRT3, hSIRT4, hSIRT5, hSIRT6 and hSIRT7 (Brachmann et al. (1995) Genes Dev. 9:2888 and Frye et al. (1999) BBRC 260:273).

“SIRT1 protein” refers to a member of the sir2 family of sirtuin deacetylases. In certain embodiments, a SIRT1 protein includes yeast Sir2 (GenBank Accession No. P53685), C. elegans Sir-2.1 (GenBank Accession No. NP_{—}501912), human SIRT1 (GenBank Accession No. NM_{—}012238 or NP_{—}036370 (or AF083106)), mouse SIRT1 (GenBank Accession No. NM_{—}019812 or NP_{—}062786), and equivalents and fragments thereof. In another embodiment, a SIRT1 protein includes a polypeptide comprising a sequence consisting of, or consisting essentially of, the amino acid sequence set forth in GenBank Accession Nos. NP_{—}036370, NP_{—}501912, NP_{—}085096, NP_{—}036369, or P53685. SIRT1 proteins include polypeptides comprising all or a portion of the amino acid sequence set forth in GenBank Accession Nos. NP_{—}036370, NP_{—}501912, NP_{—}085096, NP_{—}036369, or P53685; the amino acid sequence set forth in GenBank Accession Nos. NP_{—}036370, NP_{—}501912, NP_{—}085096, NP_{—}036369, or P53685 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. NP_{—}036370, NP_{—}501912, NP_{—}085096, NP_{—}036369, or P53685, and functional fragments thereof. Polypeptides of the invention also include homologs (e.g., orthologs and paralogs), variants, or fragments, of GenBank Accession Nos. NP_{—}036370, NP_{—}501912, NP_{—}085096, NP_{—}036369, or P53685.

As used herein “SIRT2 protein”, “SIRT3 protein”, “SIRT4 protein”, SIRT5 protein”, “SIRT6 protein”, and “SIRT7 protein” refer to other mammalian, e.g. human, sirtuin deacetylase proteins that are homologous to SIRT1 protein, particularly in the approximately 275 amino acid conserved catalytic domain. For example, “SIRT3 protein” refers to a member of the sirtuin deacetylase protein family that is homologous to SIRT1 protein. In certain embodiments, a SIRT3 protein includes human SIRT3 (GenBank Accession No. AAH01042, NP_{—}036371, or NP_{—}001017524) and mouse SIRT3 (GenBank Accession No. NP_{—}071878) proteins, and equivalents and fragments thereof. In certain embodiments, a SIRT4 protein includes human SIRT4 (GenBank Accession No. NM_{—}012240 or NP_{—}036372). In certain embodiments, a SIRT5 protein includes human SIRT5 (GenBank Accession No. NM_{—}012241 or NP_{—}036373). In certain embodiments, a SIRT6 protein includes human SIRT6 (GenBank Accession No. NM_{—}016539 or NP_{—}057623). In another embodiment, a SIRT3 protein includes a polypeptide comprising a sequence consisting of, or consisting essentially of, the amino acid sequence set forth in GenBank Accession Nos. AAH01042, NP_{—}036371, NP_{—}001017524, or NP_{—}071878. SIRT3 proteins include polypeptides comprising all or a portion of the amino acid sequence set forth in GenBank Accession AAH01042, NP_{—}036371, NP_{—}001017524, or NP_{—}071878; the amino acid sequence set forth in GenBank Accession Nos. AAH01042, NP_{—}036371, NP_{—}001017524, or NP_{—}071878 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. AAH01042, NP_{—}036371, NP_{—}001017524, or NP_{—}071878, and functional fragments thereof. Polypeptides of the invention also include homologs (e.g., orthologs and paralogs), variants, or fragments, of GenBank Accession Nos. AAH01042, NP_{—}036371, NP_{—}001017524, or NP_{—}071878. In certain embodiments, a SIRT3 protein includes a fragment of SIRT3 protein that is produced by cleavage with a mitochondrial matrix processing peptidase (MPP) and/or a mitochondrial intermediate peptidase (MIP).

The terms “systemic administration” and “administered systemically,” are art-recognized and refer to the administration of a subject composition, therapeutic or other material enterally or parenterally.

The term “therapeutic agent” is art-recognized and refers to any biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human.

The term “therapeutic effect” is art-recognized and refers to a beneficial local or systemic effect in animals, particularly mammals, and more particularly humans, caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of skill in the art. For example, certain compositions described herein may be administered in a sufficient amount to produce a desired effect at a reasonable benefit/risk ratio applicable to such treatment.

“Treating” a condition or disease refers to curing as well as ameliorating at least one symptom of the condition or disease.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₄ straight chained or branched alkyl group is also referred to as a “lower alkyl” group.

The terms “alkenyl” (“alkene”) and “alkynyl” (“alkane”) refer to unsaturated aliphatic groups analogous in length and possible substitution to the alkyl groups described above, but that contain at least one double or triple bond respectively.

The term “aromatic carbocycle” refers to an aromatic hydrocarbon ring system containing at least one aromatic ring. The ring may be fused or otherwise attached to other aromatic carbocyclic rings or non-aromatic carbocyclic rings. Examples of aromatic carbocyclegroups include carbocyclic aromatic groups such as phenyl, naphthyl, and anthracyl.

“Azabicyclo” refers to a bicyclic molecule that contains a nitrogen atom in the ring skeleton. The two rings of the bicycle may be fused at two mutually bonded atoms, e.g., indole, across a sequence of atoms, e.g., azabicyclo[2.2.1]heptane, or joined at a single atom, e.g., spirocycle.

“Bicycle” or “bicyclic” refers to a two-ring system in which one, two or three or more atoms are shared between the two rings. Bicycle includes fused bicycles in which two adjacent atoms are shared by each of the two rings, e.g., decalin, indole. Bicycle also includes spiro bicycles in which two rings share a single atom, e.g., spiro[2.2]pentane, 1-oxa-6-azaspiro[3.4]octane. Bicycle further includes bridged bicycles in which at least three atoms are shared between two rings, e.g., norbornane.

“Bridged bicycle” compounds are bicyclic ring systems, in which at least three atoms are shared by both rings of the system, i.e., they include at least one bridge of one or more atoms connecting two bridgehead atoms. Bridged azabicyclo refers to a bridged bicyclic molecule that contains a nitrogen atom in at least one of the rings.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from non-aromatic and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from non-aromaticaromatic rings. In an exemplary embodiment, an aromatic ring, e.g., phenyl, may be fused to a non-aromatic or aromatic ring, e.g., cyclohexane, cyclopentane, or cyclohexene. Any combination of non-aromtatic and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated (non-aromatic). Typically, a cycloalkyl group has from 3 to about 10 carbon atoms, more typically 3 to 8 carbon atoms unless otherwise defined. A “cycloalkenyl” group is a cyclic hydrocarbon containing one or more double bonds.

A “halogen” designates F, Cl, Br or I.

A “halogen-substitution” or “halo” substitution designates replacement of one or more hydrogens with F, Cl, Br or I.

The term “heteroaryl” or “aromatic heterocycle” includes substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes ring systems having one or two rings wherein at least one of the rings is heteroaromatic, e.g., the other cyclic rings can be cycloalkyl, cycloalkenyl, cycloalkynyl, aromatic carbocycle, heteroaryl, and/or heterocyclyl. Heteroaryl groups include, for example, pyrrole, furan, thiophene, imidazole, oxazole, thiazole, pyrazole, pyridine, pyrazine, pyridazine, and pyrimidine.

The terms “heterocycle”, and “heterocyclic”, as used herein, refers to a non-aromatic or aromatic ring comprising one or more heteroatoms selected from, for example, N, O, B and S atoms, preferably N, O, or S. The term “heterocycle” includes both “aromatic heterocycles” and “non-aromatic heterocycles.” Heterocycles include 4-7 membered monocyclic and 8-12 membered bicyclic rings. Heterocycle includes bicyclic molecules in which one, two or three or more atoms are shared between the two rings. Each ring of a bicyclic heterocycle may be selected from non-aromatic and aromatic rings. The term “fused heterocycle” refers to a bicyclic heterocycle in which each of the rings shares two adjacent atoms with the other ring. Each ring of a fused heterocycle may be selected from non-aromatic and aromatic rings. In an exemplary embodiment, an aromatic ring, e.g., pyridyl, may be fused to a non-aromatic or aromatic ring, e.g., cyclohexane, cyclopentane, pyrrolidine, 2,3-dihydrofuran or cyclohexene. “Heterocycle” groups include, for example, piperidine, piperazine, pyrrolidine, morpholine, pyrimidine, benzofuran, indole, quinoline, lactones, and lactams. Exemplary “fused heterocycles” include benzodiazepine, indole, quinoline, purine, and 4,5,6,7-tetrahydrobenzo[d]thiazole. “Heterocycles” may be substituted at any one or more positions capable of bearing a hydrogen atom.

“Monocyclic rings” include 5-7 membered aromatic carbocycle or heteroaryl, 3-7 membered cycloalkyl or cycloalkenyl, and 5-7 membered non-aromatic heterocyclyl. Exemplary monocyclic groups include substituted or unsubstituted heterocycles or carbocycles such as thiazolyl, oxazolyl, oxazinyl, thiazinyl, dithianyl, dioxanyl, isoxazolyl, isothiazolyl, triazolyl, furanyl, tetrahydrofuranyl, dihydrofuranyl, pyranyl, tetrazolyl, pyrazolyl, pyrazinyl, pyridazinyl, imidazolyl, pyridinyl, pyrrolyl, dihydropyrrolyl, pyrrolidinyl, piperidinyl, piperazinyl, pyrimidinyl, morpholinyl, tetrahydrothiophenyl, thiophenyl, cyclohexyl, cyclopentyl, cyclopropyl, cyclobutyl, cycloheptanyl, azetidinyl, oxetanyl, thiiranyl, oxiranyl, aziridinyl, and thiomorpholinyl.

As used herein, “substituted” means substituting a hydrogen atom in a structure with an atom or molecule other than hydrogen. A substitutable atom such as a “substitutable nitrogen” is an atom that bears a hydrogen atom in at least one resonance form. The hydrogen atom may be substituted for another atom or group such as a CH₃ or an OH group. For example, the nitrogen in a piperidine molecule is substitutable if the nitrogen is bound to a hydrogen atom. If, for example, the nitrogen of a piperidine is bound to an atom other than hydrogen, the nitrogen is not substitutable. An atom that is not capable of bearing a hydrogen atom in any resonance form is not substitutable.

Combinations of substituents and variables envisioned by this invention are only those that result in the formation of stable compounds. As used herein, the term “stable” refers to compounds that possess stability sufficient to allow manufacture and that maintain the integrity of the compound for a sufficient period of time to be useful for the purposes detailed herein.

The compounds disclosed herein also include partially and fully deuterated variants. In certain embodiments, deuterated variants may be used for kinetic studies. One of skill in the art can select the sites at which such deuterium atoms are present.

Also included in the present invention are salts, particularly pharmaceutically acceptable salts, of the compounds described herein. The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quarternary nitrogen, can form a salt with an appropriate counterion (e.g., a halide such as bromide, chloride, or fluoride, particularly bromide).

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

Certain compounds of the present invention may exist in particular geometric or stereoisomeric forms. The present invention contemplates all such compounds, including cis- and trans-isomers, (R)- and (S)-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

The term “steroisomer” as used herein is art-recognized and refers to any of two or more isomers that have the same molecular constitution and differ only in the three-dimensional arrangement of their atomic groupings in space. When used herein to describe a compounds or genus of compounds, stereoisomer includes any portion of the compound or the compound in its entirety. For example, diastereomers and enantiomers are stereoisomers.

The term “tautomer” as used herein is art-recognized and refers to any one of the possible alternative structures that may exist as a result of tautomerism, which refers to a form of constitutional isomerism in which a structure may exist in two or more constitutional arrangements, particularly with respect to the position of hydrogens bonded to oxygen. When used herein to describe a compound or genus of compounds, it is further understood that a “tautomer” is readily interconvertible and exists in equilibrium. For example, keto and enol tautomers exist in proportions determined by the equilibrium position for any given condition, or set of conditions:

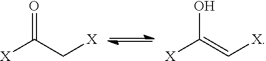

Compounds of the invention, including novel compounds of the invention, can also be used in the methods described herein.

The compounds and salts thereof described herein can also be present as the corresponding hydrates (e.g., hemihydrate, monohydrate, dihydrate, trihydrate, tetrahydrate) or solvates. Suitable solvents for preparation of solvates and hydrates can generally be selected by a skilled artisan.

The compounds and salts thereof can be present in amorphous or crystalline (including co-crystalline and polymorph) forms.

Sirtuin-modulating compounds of the invention advantageously modulate the level and/or activity of a sirtuin protein, particularly the deacetylase activity of the sirtuin protein.

According to another embodiment, the present invention provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### 2. Compounds

In one aspect, compounds of the present invention, or compositions comprising compounds of the present invention that decrease the level and/or activity of a sirtuin protein may be used for treating and/or preventing diseases and disorders including cancers, neurodegenerative diseases, and inflammatory disorders and conditions. Compounds disclosed herein may be suitable for use in pharmaceutical compositions and/or one or more methods disclosed herein.

In one embodiment, sirtuin-modulating compounds of the invention are represented by Structural Formula (I):

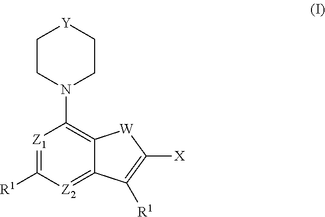

or a salt thereof wherein:

each of Z₁ and Z₂ is independently selected from N and CR¹, wherein:

at least one of Z₁ and Z₂ is N;

each R¹ is independently selected from hydrogen, halo, C₁-C₄ straight chain or branched alkyl, halo substituted C₁-C₄ straight chain or branched alkyl, —O—C₁-C₄ straight chain or branched alkyl, —O-halo-substituted C₁-C₄ straight chain or branched alkyl, C₁-C₄ alkoxy-substituted C₁-C₄ straight chain or branched alkyl, and hydroxy-substituted C₁-C₄ straight chain or branched alkyl;

W is selected from S and O;

X is selected from —C(═O)—NH₂, —S(═O)₂—NH₂, —C(═NH)—NH₂, —C(═O)NHOH, —C(═S)—NH₂, —S(═O)—NH₂ and —SO₃H;

Y is selected from CHR², CR²—(C₁-C₄ straight chain or branched alkyl)-NR³R³, CH—(C₁-C₄ straight chain or branched alkyl)-R², CH—(C₁-C₄ straight chain or branched alkyl)-NR³R³, CH—(C₁-C₄ straight chain or branched alkyl)-NH—C(═O)—R², CH—(C₁-C₄ straight chain or branched alkyl)-NH—C(═S)—R², CH—(C₁-C₄ straight chain or branched alkyl)-C(═O)—NR³R³, N—(C₁-C₄ straight chain or branched alkyl)-NH—C(═O)—R², N—(C₁-C₄ straight chain or branched alkyl)-NH—C(═S)—R², N—(C₁-C₄ straight chain or branched alkyl)-NR³R³, N—(C₁-C₄ straight chain or branched alkyl)-R², and C-linked 5-6 membered saturated heterocycle;

R² is selected from 5- to 6-membered saturated or unsaturated carbocycle or heterocycle, —OH, —O—(C₁-C₄ straight chain or branched alkyl), —C₁-C₄ straight chain or branched alkyl, —S(═O)₂—CH₃, —C(═O)—O—(C₁-C₄ straight chain or branched alkyl), —C(═O)—(C₁-C₄ straight chain or branched alkyl), and when R² is a 5- to 6-membered saturated or unsaturated carbocycle or heterocycle, R² is also optionally substituted with one or more substituents independently selected from halo, —C₁-C₄ straight chain or branched alkyl, —C(═O)—NH—(C₁-C₄ straight chain or branched alkyl), —C(═O)—O—(C₁-C₄ straight chain or branched alkyl), —C(═O)—O—(C₁-C₄ straight chain or branched alkyl), —C(═O)—OH, —O—PO₃H₂ and —C(═O)—NH—(C₁-C₄ straight chain or branched alkyl)-NH₂; and

R³ is independently selected from hydrogen, —C₁-C₄ straight chain or branched alkyl, —C(═O)-(5- to 6-membered saturated carbocycle or heterocycle) and —S(═O)₂—CH₃; or

two R³ bound to the same nitrogen are taken together with the nitrogen atom to form a 5- to 6-membered saturated heterocycle optionally comprising one or two additional heteroatoms selected from N, S, S(═O), S(═O)₂, and O, wherein the heterocycle is optionally substituted at any carbon atom with one or more of —OH, ═O, halo, —C₁-C₄ straight chain or branched alkyl, fluoro-substituted C₁-C₄ straight chain or branched alkyl, hydroxy-substituted C₁-C₄ straight chain or branched alkyl, alkoxy-substituted C₁-C₄ straight chain or branched alkyl, —C(═O)—C₁-C₄ straight chain or branched alkyl, and optionally substituted at any substitutable nitrogen atom with —C₁-C₄ straight chain or branched alkyl, —C(═O)—C₁-C₄ straight chain or branched alkyl,hydroxy-substituted C₁-C₄ straight chain or branched alkyl, alkoxy-substituted C₁-C₄ straight chain or branched alkyl, or halo-substituted C₁-C₄ straight chain or branched alkyl; wherein

when Y is a C-linked 5- to 6-membered heterocycle, it is further optionally substituted at any carbon atom with one or more of —C(═O)—R², —OH, ═O, halo, —C₁-C₄ straight chain or branched alkyl, fluoro-substituted C₁-C₄ straight chain or branched alkyl, hydroxy-substituted C₁-C₄ straight chain or branched alkyl, alkoxy-substituted C₁-C₄ straight chain or branched alkyl, and optionally substituted at any substitutable nitrogen atom with —C₁-C₄ straight chain or branched alkyl, —C(═O)—R², hydroxy-substituted C₁-C₄ straight chain or branched alkyl, alkoxy-substituted C₁-C₄ straight chain or branched alkyl, or halo-substituted C₁-C₄ straight chain or branched alkyl.

In certain embodiments, the compounds of Structural Formula (I) is represented by Structural Formula (Ia):

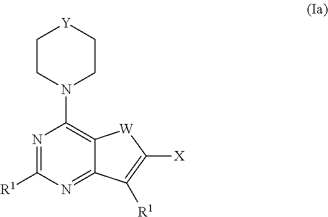

or salt thereof.

In certain embodiments, the compounds of Structural Formula (I) is represented by Structural Formula (Ib):

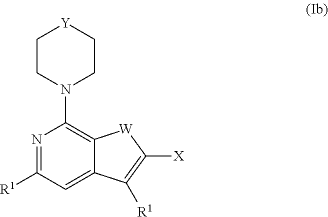

or salt thereof.

In certain embodiments, the compounds of Structural Formula (I) is represented by Structural Formula (Ic):

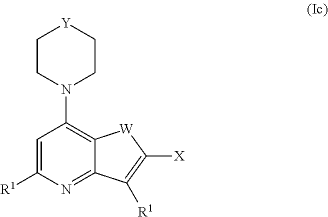

or salt thereof.

In certain embodiments, the compounds of Structural Formula (I) (including all of (Ia), (Ib), and (Ic)) are characterized by W being S.

In certain embodiments, the compounds of Structural Formula (I) (including all of (Ia), (Ib), and (Ic)) are characterized by W being O.

In certain embodiments, the compounds of Structural Formula (I) (including all of (Ia), (Ib), and (Ic)) are characterized by X being —C(═O)—NH₂.

In certain embodiments, the compounds of Structural Formula (I) (including all of (Ia), (Ib), and (Ic)) are characterized by having Y selected from CH—(C₁-C₄ straight chain or branched alkyl)-NH—C(═O)—R², CH—(C₁-C₄ straight chain or branched alkyl)-NR³R³, N—(C₁-C₄ straight chain or branched alkyl)-NH—C(═O)—R², N—(C₁-C₄ straight chain or branched alkyl)-NR³R³, CH—(C₁-C₄ straight chain or branched alkyl)-R², and CH—(C₁-C₄ straight chain or branched alkyl)-NH—C(═S)—R².

In particular embodiments, the compounds of Structural Formula (I) (including all of (Ia), (Ib), and (Ic)), Y is CH—(C₁-C₄ straight chain or branched alkyl)-NH—C(═O)—R². Examples of these embodiments include:

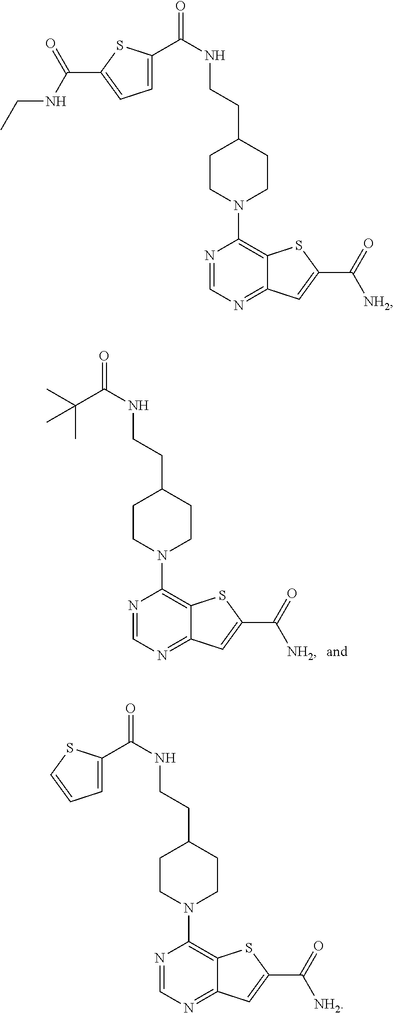 [SMILES: CCNC(=O)c1ccc(C(=O)NCCC2CCN(c3ncnc4cc(C(N)=O)sc34)CC2)s1 ; CC(C)(C)C(=O)NCCC1CCN(c2ncnc3cc(C(N)=O)sc23)CC1 ; NC(=O)c1cc2ncnc(N3CCC(CCNC(=O)c4cccs4)CC3)c2s1]

In further embodiments, the compounds of Structural Formula (I) (including all of (Ia), (Ib), and (Ic)), Y is CH—(C₁-C₄ straight chain or branched alkyl)-NR³R³. Examples of these embodiments include:

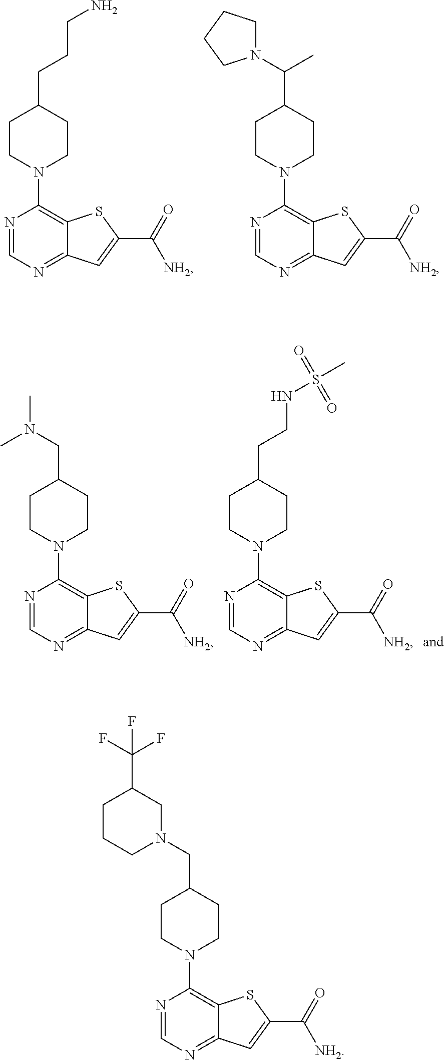

In further embodiments, the compounds of Structural Formula (I) (including all of (Ia), (Ib), and (Ic)), Y is N—(C₁-C₄ straight chain or branched alkyl)-NH—C(═O)—R². Examples of these embodiments include:

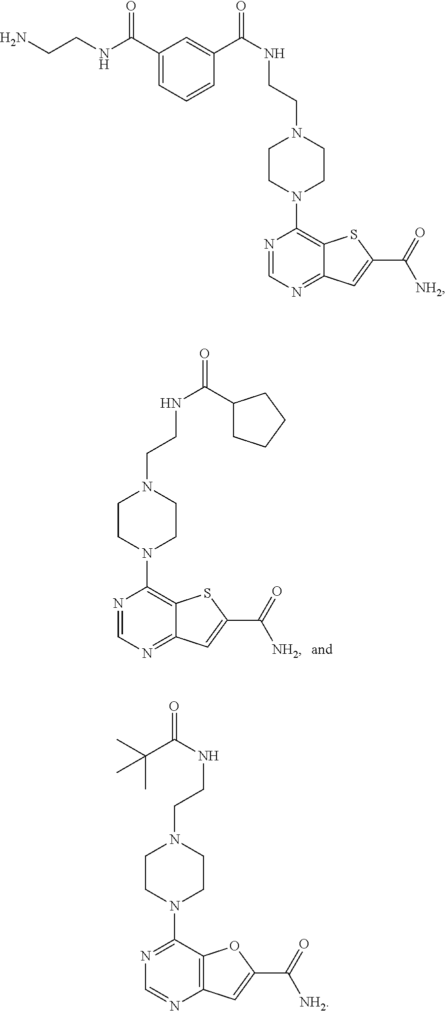 [SMILES: NCCNC(=O)c1cccc(C(=O)NCCN2CCN(c3ncnc4cc(C(N)=O)sc34)CC2)c1 ; NC(=O)c1cc2ncnc(N3CCN(CCNC(=O)C4CCCC4)CC3)c2s1 ; CC(C)(C)C(=O)NCCN1CCN(c2ncnc3cc(C(N)=O)oc23)CC1]

In further embodiments, the compounds of Structural Formula (I) (including all of (Ia), (Ib), and (Ic)), Y is N—(C₁-C₄ straight chain or branched alkyl)-NR³R³. One example of these embodiments is:

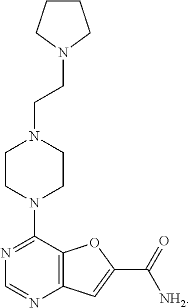 [SMILES: NC(=O)c1cc2ncnc(N3CCN(CCN4CCCC4)CC3)c2o1]

In further embodiments, the compounds of Structural Formula (I) (including all of (Ia), (Ib), and (Ic)), Y is CH—(C₁-C₄ straight chain or branched alkyl)-R². Examples of these embodiments include:

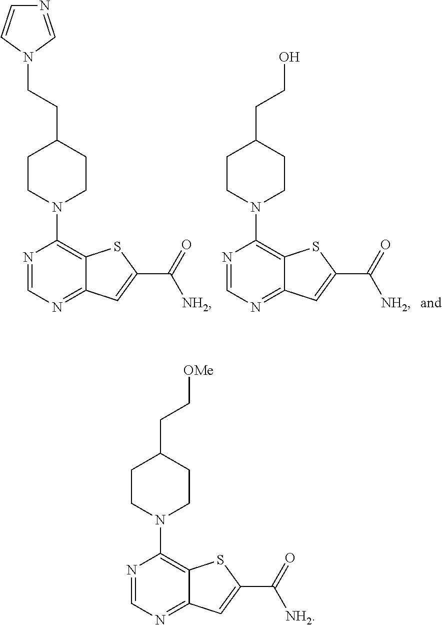

In further embodiments, the compounds of Structural Formula (I) (including all of (Ia), (Ib), and (Ic)), Y is CH—(C₁-C₄ straight chain or branched alkyl)-NH—C(═S)—R². One example of these embodiments is:

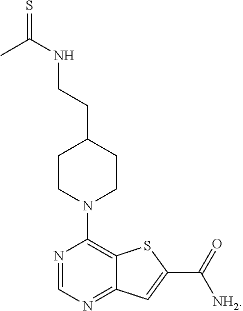 [SMILES: CC(=S)NCCC1CCN(c2ncnc3cc(C(N)=O)sc23)CC1]

In still further embodiments, the compounds of Structural Formula (I) (including all of (Ia), (Ib), and (Ic)), Y is CHR². One example of these embodiments is:

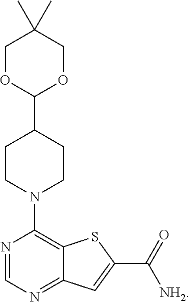 [SMILES: CC1(C)COC(C2CCN(c3ncnc4cc(C(N)=O)sc34)CC2)OC1]

In further embodiments, the compounds of Structural Formula (I) (including all of (Ia), (Ib), and (Ic)), Y is a C-linked heterocycle. Examples of these embodiments include:

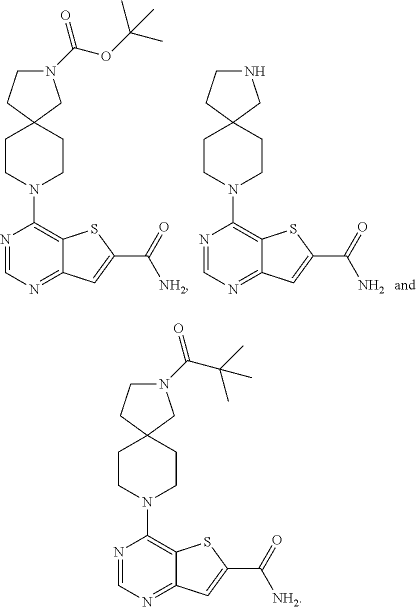

In particular embodiments of the above, Y is CH—(C₁-C₄ straight chain or branched alkyl)-NH—C(═O)—R² or N—(C₁-C₄ straight chain or branched alkyl)-NH—C(═O)—R².

In further embodiments of the above, R² is selected from a 5- to 6-membered saturated or unsaturated carbocycle or heterocycle, —C₁-C₄ straight chain or branched alkyl, —O—(C₁-C₄ straight chain or branched alkyl), and —OH.

In certain embodiments of the above, R³ is selected from —C₁-C₄ straight chain or branched alkyl and —S(═O)₂—CH₃.

In further embodiments of the above, two R³ bound to the same nitrogen are taken together with the nitrogen atom to form an optionally substituted 5- to 6-membered saturated heterocycle.

The compounds of the invention, including novel compounds of the invention, can also be used in the methods described herein. The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms. Sirtuin-modulating compounds of the invention advantageously modulate the level and/or activity of a sirtuin protein, particularly the deacetylase activity of the sirtuin protein.

Separately or in addition to the above properties, certain sirtuin-modulating compounds of the invention do not substantially have one or more of the following activities: inhibition of PI3-kinase, inhibition of aldoreductase, inhibition of tyrosine kinase, transactivation of EGFR tyrosine kinase, coronary dilation, or spasmolytic activity, at concentrations of the compound that are effective for modulating the deacetylation activity of a sirtuin protein (e.g., such as a SIRTI and/or a SIRT3 protein).

In further embodiments, the invention provides pharmaceutical compositions comprising any of the above compounds or above-described embodiments and a pharmaceutically acceptable carrier or diluent. In certain embodiments, the pharmaceutical composition further comprises an additional active agent. Examples of additional active agents include anti-inflammatory agents, chemotherapeutic agents, analgesics, antimicrobial agents, antifungal agents, antibiotics, vitamins, antioxidants, and sunblock agents commonly found in sunscreen formulations including, but not limited to, anthranilates, benzophenones particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In certain embodiments, the invention provides methods for treating a subject suffering from a neurodegenerative disorder, or cancer comprising administering to the subject in need thereof a pharmaceutical composition of the invention, i.e., a pharmaceutical compositions comprising any of the above compounds or above-described embodiments and a pharmaceutically acceptable carrier or diluent

In further embodiments, the invention provides any of the above-described compounds or embodiments for use as a pharmaceutical.

In certain embodiments, the invention provides methods for inhibiting sirtuin activity in a cell or lysate. In particular embodiments, the sirtuin activity inhibited is a SIRT1, a SIRT2, and/or a SIRT3 sirtuin activity.

In further embodiments, the invention provides methods of determining whether a process, signal, or effect detected in a cell or cell lysate is sirtuin-dependent. The methods comprise the step of comparing the presence, level, or amount of the process, signal, or effect in the presence of a compound of the invention to the presence, level, or amount of process, signal, or effect in the absence of the compound of the invention, wherein a change in the presence, level, or amount of the process, signal, or effect in the presence of the compound as compared to in the absence of the compound indicates that the process, signal, or effect is sirtuin-dependent.

In another embodiment, the invention provides methods of detecting sirtuin-dependence in a biological signal. The methods comprise the step of comparing the biological signal in the presence of a sirtuin inhibitor compound of the invention to the biological signal in the absence of the sirtuin inhibitory compound, wherein an increase or decrease in the biological signal in the presence of the sirtuin inhibitor compound of the invention as compared to the biological signal in the absence of the sirtuin inhibitor compound of the invention indicates that the biological signal is sirtuin-dependent.

Any of the above-described compounds or embodiments may be used in these methods of the invention.

In certain embodiments of these methods of the invention, the sirtuin dependence is selected from one or more of SIRT1-dependent, SIRT2-dependent, and SIRT3-dependent.

The invention includes pharmaceutical compositions comprising of any of the compounds of Structural Formulas (I), (Ia), (Ib) or (Ic), or as otherwise set forth above. The pharmaceutical composition of the compound of Structural Formulas (I), (Ia), (Ib), or (Ic) may comprise one or more pharmaceutically acceptable carriers or diluents. The pharmaceutical composition of the compound of Structural Formulas (I), (Ia), (Ib), or (Ic) may comprise a second/additional active agent.

Compounds of the present invention can also be used in the methods described herein. In particular embodiments, the compounds of the present invention may be used for treating a subject suffering from or susceptible to a metabolic syndrome, neurodegenerative disorder, inflammatory disorder, or complications thereof, comprising administering to the subject in need thereof a composition comprising a compound of Structural Formulas (I), (Ia), (Ib), or (Ic). In particular embodiments the compounds of the present invention may be used for treating a subject suffering from or susceptible to a metabolic syndrome, neurodegenerative disorder, inflammatory disorder, or complications thereof, comprising administering to the subject in need thereof a composition comprising a compound of Structural Formulas (I), (Ia), (Ib), or (Ic), further comprising administering a second/additional active agent.

In any of the preceding embodiments, a C₁-C₄ alkoxy-substituted group may include one or more alkoxy substituents such as one, two or three methoxy groups or a methoxy group and an ethoxy group, for example. Exemplary C₁-C₄ alkoxy substituents include methoxy, ethoxy, isopropoxy, and tert-butoxy.

In any of the preceding embodiments, a hydroxy-substituted group may include one or more hydroxy substituents, such as two or three hydroxy groups.

In any of the preceding embodiments, a “halo-substituted” group includes from one halo substituent up to perhalo substitution. Exemplary halo-substituted C₁-C₄ alkyl includes CFH₂, CClH₂, CBrH₂, CF₂H, CCl₂H, CBr₂H, CF₃, CCl₃, CBr₃, CH₂CH₂F, CH₂CH₂Cl, CH₂CH₂Br, CH₂CHF₂, CHFCH₃, CHClCH₃, CHBrCH₃, CF₂CHF₂, CF₂CHCl₂, CF₂CHBr₂, CH(CF₃)₂, and C(CF₃)₃. Perhalo-substituted C₁-C₄ alkyl, for example, includes CF₃, CCl₃, CBr₃, CF₂CF₃, CCl₂CF₃ and CBr₂CF₃.

Certain compounds of the present invention may exist in particular geometric or stereoisomeric forms. The present invention contemplates all such compounds, including cis- and trans-isomers, (R)- and (S)-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

The compounds and salts thereof described herein can also be present as the corresponding hydrates (e.g., hemihydrate, monohydrate, dihydrate, trihydrate, tetrahydrate) or solvates. Suitable solvents for preparation of solvates and hydrates can generally be selected by a skilled artisan.

The compounds and salts thereof can be present in amorphous or crystalline (including co-crystalline and polymorph) forms.

### 3. Exemplary Uses

### Cell Permeability and Protein Binding Affinity of Sirtuin Modulating Compounds

In an exemplary embodiment, a therapeutic compound may traverse the cytoplasmic membrane of a cell. For example, a compound may have a cell-permeability of at least about 20%, 50%, 75%, 80%, 90% or 95%.

In certain embodiments, a sirtuin-modulating compound may have a binding affinity for a sirtuin protein of about 10⁻⁹M, 10⁻¹⁰M, 10⁻¹¹ M, 10⁻¹²M or less. A sirtuin-modulating compound may reduce (activator) or increase (inhibitor) the apparent Km of a sirtuin protein for its substrate or NAD⁺ (or other cofactor) by a factor of at least about 2, 3, 4, 5, 10, 20, 30, 50 or 100. In certain embodiments, Km values are determined using the mass spectrometry assay described herein. A sirtuin-modulating compound may increase or decrease the Vmax of a sirtuin protein by a factor of at least about 2, 3, 4, 5, 10, 20, 30, 50 or 100. A sirtuin-modulating compound may have an IC₅₀ for modulating the deacetylase activity of a SIRT1 and/or SIRT3 protein of less than about 1 nM, less than about 10 nM, less than about 100 nM, less than about 1 μM, less than about 10 μM, less than about 100 μM, or from about 1-10 nM, from about 10-100 nM, from about 0.1-1 μM, from about 1-10 μM or from about 10-100 μM. A sirtuin-modulating compound may modulate the deacetylase activity of a SIRT1, SIRT2 and SIRT3 protein by a factor of at least about 5, 10, 20, 30, 50, or 100, as measured in a cellular assay or in a cell based assay.

### Sirtuin Modulation

In certain aspects, the invention provides methods for modulating the level and/or activity of a sirtuin protein and methods of use thereof.

In certain embodiments, the invention provides methods for using sirtuin-modulating compounds wherein the sirtuin-modulating compounds inhibit a sirtuin protein, e.g., decreases the activity of a sirtuin protein. Sirtuin-inhibiting compounds that decrease the activity of a sirtuin protein may be useful for a variety of therapeutic applications including, for example, decreasing the lifespan of a cell, and treating and/or preventing a wide variety of diseases and disorders including, for example, diseases or disorders related to aging or stress, diabetes, obesity, neurodegenerative diseases, cardiovascular disease, blood clotting disorders, inflammation, and cancer. The methods comprise administering to a subject in need thereof a pharmaceutically effective amount of a sirtuin-modulating compound, e.g., a sirtuin-modulating compound.

In certain embodiments, the sirtuin-modulating compounds described herein may be taken alone or in combination with other compounds. In certain embodiments, a mixture of two or more sirtuin-modulating compounds may be administered to a subject in need thereof. In another embodiment, a sirtuin-modulating compound that decreases the level and/or activity of a sirtuin protein may be administered with one or more of the following compounds: sirtinol; salermide; EX-527; suramin; cambinol; splitomicin; NF023 (a G-protein antagonist); NF279 (a purinergic receptor antagonist); Trolox (6-hydroxy-2,5,7,8,tetramethylchroman-2-carboxylic acid); (−)-epigallocatechin (hydroxy on sites 3,5,7,3′,4′,5′); (−)-epigallocatechin gallate (Hydroxy sites 5,7,3′,4′,5′ and gallate ester on 3); cyanidin chloride (3,5,7,3′,4′-pentahydroxyflavylium chloride); delphinidin chloride (3,5,7,3′,4′,5′-hexahydroxyflavylium chloride); myricetin (cannabiscetin; 3,5,7,3′,4′,5′-hexahydroxyflavone); 3,7,3′,4′,5′-pentahydroxyflavone; gossypetin (3,5,7,8,3′,4′-hexahydroxyflavone); (S)-2-((S)-2-((S)-2-acetamidopropanamido)-6-ethanethioamidohexanamido)propanoic acid (Compound 5); 2-((3-(3-fluorophenethoxyl)phenyl)amino)benzamide (Compound 7); (S)-8-bromo-6-chloro-2-pentylchroman-4-one (Compound 8).

In an exemplary embodiment, a sirtuin-modulating compound that decreases the level and/or activity of a sirtuin protein may be administered in combination with nicotinic acid or nicotinamide riboside. In another embodiment, a sirtuin-modulating compound that decreases the level and/or activity of a sirtuin protein may be administered with one or more of the following compounds: nicotinamide (NAM), resveratrol, butein, fisetin, piceatannol, quercetin; niacinamide, valproic acid, sodium butyrate, vorinostat, belinostat, panobinostat, entinostat, mocetinostat, romidepsin, abexinostat, resminostat, givinostat, quisinostat, SB939, CUDC-101, AR-42, CHR-2845, CHR-3996, 4SC-202, CG200745, ACY-1215, ME-344, kevetrin, sulforaphane, and trichostatin A. In yet another embodiment, one or more sirtuin-modulating compounds may be administered with one or more therapeutic agents for the treatment or prevention of various diseases, including, for example, cancer, diabetes, neurodegenerative diseases, cardiovascular disease, blood clotting, inflammation, flushing, obesity, aging, stress, etc. In various embodiments, combination therapies comprising a sirtuin-modulating compound may refer to (1) pharmaceutical compositions that comprise one or more sirtuin-modulating compounds in combination with one or more therapeutic agents (e.g., one or more therapeutic agents described herein); and (2) co-administration of one or more sirtuin-modulating compounds with one or more therapeutic agents wherein the sirtuin-modulating compound and therapeutic agent have not been formulated in the same compositions (but may be present within the same kit or package, such as a blister pack or other multi-chamber package; connected, separately sealed containers (e.g., foil pouches) that can be separated by the user; or a kit where the compound(s) and other therapeutic agent(s) are in separate vessels). When using separate formulations, the sirtuin-modulating compound may be administered simultaneous with, intermittent with, staggered with, prior to, subsequent to, or combinations thereof, the administration of another therapeutic agent.

In certain embodiments, methods for reducing, preventing or treating diseases or disorders using a sirtuin-modulating compound may also comprise increasing the protein level of a sirtuin, such as human SIRT1, SIRT2 and SIRT3, or homologs thereof. Increasing a sirtuin protein level can be achieved according to methods known in the art.

Methods for modulating sirtuin protein levels also include methods for modulating the transcription of genes encoding sirtuins, methods for stabilizing/destabilizing the corresponding mRNAs, and other methods known in the art.

### Cell Death/Cancer and Viral Infections

Sirtuin-modulating compounds may also be used for treating and/or preventing cancer. In certain embodiments, sirtuin-inhibiting compounds that decrease the level and/or activity of a sirtuin protein may be used for treating and/or preventing cancer. Exemplary cancers that may be treated using a sirtuin-modulating compound are those of the brain and kidney; hormone-dependent cancers including breast, prostate, testicular, and ovarian cancers; lymphomas, and leukemias. In cancers associated with solid tumors, a modulating compound may be administered directly into the tumor. Cancer of blood cells, e.g., leukemia, can be treated by administering a modulating compound into the blood stream or into the bone marrow. Benign cell growth, e.g., warts, can also be treated.

Chemotherapeutic agents may be co-administered with modulating compounds described herein as having anti-cancer activity, e.g., compounds that induce apoptosis or compounds that render cells sensitive to stress. Chemotherapeutic agents may be used by themselves with a sirtuin-modulating compound described herein as inducing cell death or reducing lifespan or increasing sensitivity to stress and/or in combination with other chemotherapeutics agents. In addition to conventional chemotherapeutics, the sirtuin-modulating compounds described herein may also be used with antisense RNA, RNAi or other polynucleotides to inhibit the expression of the cellular components that contribute to unwanted cellular proliferation.

Combination therapies comprising sirtuin-modulating compounds and a conventional chemotherapeutic agent may be advantageous over combination therapies known in the art because the combination allows the conventional chemotherapeutic agent to exert greater effect at lower dosage. In a preferred embodiment, the inhibitory concentration (IC₅₀) for a chemotherapeutic agent, or combination of conventional chemotherapeutic agents, when used in combination with a sirtuin-modulating compound is at least 2 fold less than the IC₅₀ for the chemotherapeutic agent alone, and even more preferably at 5 fold, 10 fold or even 25 fold less. Conversely, the therapeutic index (TI) for such chemotherapeutic agent or combination of such chemotherapeutic agent when used in combination with a sirtuin-modulating compound described herein can be at least 2 fold greater than the TI for conventional chemotherapeutic regimen alone, and even more preferably at 5 fold, 10 fold or even 25 fold greater.

Sirtuin-inhibiting compounds that decrease the level and/or activity of a sirtuin protein may be administered to subjects who have recently received or are likely to receive a dose of radiation or toxin. In certain embodiments, the dose of radiation or toxin is received as part of a work-related or medical procedure, e.g., administered as a prophylactic measure. In another embodiment, the radiation or toxin exposure is received unintentionally. In such a case, the compound is preferably administered as soon as possible after the exposure to inhibit apoptosis and the subsequent development of acute radiation syndrome.

Methods of treating cancers with sirtuin-inhibiting agents have been described. For example: US 2011/0092695 describes the use of SIRT1 inhibitors to treat cancer, in particular for preventing chemoresistance or treating chronic myelogenous leukemia (CML); WO 2012/135149 describes the use of SIRT1 inhibitor to effectively reactivate p53 and thereby treat abnormal cell growth such as cancers; WO 2008/082646 describes the use sirtuin inhibitors to activate methylation silenced genes, including tumor suppressor genes (e.g., frizzled related proteins, p53, E-cadherin, mismatch repair genes, and cellular retinol binding protein-I) for the purpose of treating diseases including cancer; and US 20110178153 describes the use of sirtuin inhibitors to treat relapsing and chemoresistant cancers.

Other diseases that can be treated by administration of sirtuin-modulating compound include viral infections such as herpes, HIV, adenovirus, and HTLV-1 associated malignant and benign disorders. Alternatively, cells can be obtained from a subject, treated ex vivo to remove certain undesirable cells, e.g., cancer cells, and administered back to the same or a different subject. WO 2012/106509 describes the use of inhibitors of two or more sirtuins to inhibit virus production.

### Neuronal Diseases/Disorders

In certain aspects, sirtuin-inhibiting compounds that decrease the level and/or activity of a sirtuin protein can be used to treat patients suffering from neurodegenerative diseases, and traumatic or mechanical injury to the central nervous system (CNS), spinal cord or peripheral nervous system (PNS). Neurodegenerative disease typically involves reductions in the mass and volume of the human brain, which may be due to the atrophy and/or death of brain cells, which are far more profound than those in a healthy person that are attributable to aging. Neurodegenerative diseases can evolve gradually, after a long period of normal brain function, due to progressive degeneration (e.g., nerve cell dysfunction and death) of specific brain regions. Alternatively, neurodegenerative diseases can have a quick onset, such as those associated with trauma or toxins. The actual onset of brain degeneration may precede clinical expression by many years. Examples of neurodegenerative diseases include, but are not limited to, Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, ocular diseases (ocular neuritis), chemotherapy-induced neuropathies (e.g., from vincristine, paclitaxel, bortezomib), diabetes-induced neuropathies and Friedreich's ataxia. Sirtuin-modulating compounds that increase the level and/or activity of a sirtuin protein can be used to treat these disorders and others as described below.

AD is a CNS disorder that results in memory loss, unusual behavior, personality changes, and a decline in thinking abilities. These losses are related to the death of specific types of brain cells and the breakdown of connections and their supporting network (e.g. glial cells) between them. The earliest symptoms include loss of recent memory, faulty judgment, and changes in personality. PD is a CNS disorder that results in uncontrolled body movements, rigidity, tremor, and dyskinesia, and is associated with the death of brain cells in an area of the brain that produces dopamine. ALS (motor neuron disease) is a CNS disorder that attacks the motor neurons, components of the CNS that connect the brain to the skeletal muscles.

HD is another neurodegenerative disease that causes uncontrolled movements, loss of intellectual faculties, and emotional disturbance. Tay-Sachs disease and Sandhoff disease are glycolipid storage diseases where GM2 ganglioside and related glycolipids substrates for β-hexosaminidase accumulate in the nervous system and trigger acute neurodegeneration.

It is well-known that apoptosis plays a role in AIDS pathogenesis in the immune system. However, HIV-1 also induces neurological disease, which can be treated with sirtuin-modulating compounds of the invention.

Neuronal loss is also a salient feature of prion diseases, such as Creutzfeldt-Jakob disease in human, BSE in cattle (mad cow disease), Scrapie Disease in sheep and goats, and feline spongiform encephalopathy (FSE) in cats. Sirtuin-modulating compounds that decrease the level and/or activity of a sirtuin protein may be useful for treating or preventing neuronal loss due to these prior diseases.

In another embodiment, a sirtuin-modulating compound that decreases the level and/or activity of a sirtuin protein may be used to treat or prevent any disease or disorder involving axonopathy. Distal axonopathy is a type of peripheral neuropathy that results from some metabolic or toxic derangement of peripheral nervous system (PNS) neurons. It is the most common response of nerves to metabolic or toxic disturbances, and as such may be caused by metabolic diseases such as diabetes, renal failure, deficiency syndromes such as malnutrition and alcoholism, or the effects of toxins or drugs. Those with distal axonopathies usually present with symmetrical glove-stocking sensori-motor disturbances. Deep tendon reflexes and autonomic nervous system (ANS) functions are also lost or diminished in affected areas.

Diabetic neuropathies are neuropathic disorders that are associated with diabetes mellitus. Relatively common conditions which may be associated with diabetic neuropathy include third nerve palsy; mononeuropathy; mononeuritis multiplex; diabetic amyotrophy; a painful polyneuropathy; autonomic neuropathy; and thoracoabdominal neuropathy.

Peripheral neuropathy is the medical term for damage to nerves of the peripheral nervous system, which may be caused either by diseases of the nerve or from the side-effects of systemic illness. Major causes of peripheral neuropathy include seizures, nutritional deficiencies, and HIV, though diabetes is the most likely cause. In an exemplary embodiment, a sirtuin-modulating compound that decreases the level and/or activity of a sirtuin protein may be used to treat or prevent multiple sclerosis (MS), including relapsing MS and monosymptomatic MS, and other demyelinating conditions, such as, for example, chronic inflammatory demyelinating polyneuropathy (CIDP), or symptoms associated therewith.

In yet another embodiment, a sirtuin-modulating compound that decreases the level and/or activity of a sirtuin protein may be used to treat trauma to the nerves, including, trauma due to disease, injury (including surgical intervention), or environmental trauma (e.g., neurotoxins, alcoholism, etc.).

Sirtuin-modulating compounds that decrease the level and/or activity of a sirtuin protein may also be useful to prevent, treat, and alleviate symptoms of various PNS disorders. The term “peripheral neuropathy” encompasses a wide range of disorders in which the nerves outside of the brain and spinal cord—peripheral nerves—have been damaged. Peripheral neuropathy may also be referred to as peripheral neuritis, or if many nerves are involved, the terms polyneuropathy or polyneuritis may be used.

PNS diseases treatable with sirtuin-modulating compounds that decrease the level and/or activity of a sirtuin protein include: diabetes, leprosy, Charcot-Marie-Tooth disease, Guillain-Barré syndrome and Brachial Plexus Neuropathies (diseases of the cervical and first thoracic roots, nerve trunks, cords, and peripheral nerve components of the brachial plexus.

In another embodiment, a sirtuin-modulating compound may be used to treat or prevent a polyglutamine disease. Exemplary polyglutamine diseases include Spinobulbar muscular atrophy (Kennedy disease), Huntington's Disease (HD), Dentatorubral-pallidoluysian atrophy (Haw River syndrome), Spinocerebellar ataxia type 1, Spinocerebellar ataxia type 2, Spinocerebellar ataxia type 3 (Machado-Joseph disease), Spinocerebellar ataxia type 6, Spinocerebellar ataxia type 7, and Spinocerebellar ataxia type 17.

In certain embodiments, the invention provides a method to treat a central nervous system cell to prevent damage in response to a decrease in blood flow to the cell. Typically the severity of damage that may be prevented will depend in large part on the degree of reduction in blood flow to the cell and the duration of the reduction. In certain embodiments, apoptotic or necrotic cell death may be prevented. In still a further embodiment, ischemic-mediated damage, such as cytotoxic edema or central nervous system tissue anoxemia, may be prevented. In each embodiment, the central nervous system cell may be a spinal cell or a brain cell.

Another aspect encompasses administrating a sirtuin-modulating compound to a subject to treat a central nervous system ischemic condition. A number of central nervous system ischemic conditions may be treated by the sirtuin-modulating compounds described herein. In certain embodiments, the ischemic condition is a stroke that results in any type of ischemic central nervous system damage, such as apoptotic or necrotic cell death, cytotoxic edema or central nervous system tissue anoxia. The stroke may impact any area of the brain or be caused by any etiology commonly known to result in the occurrence of a stroke. In one alternative of this embodiment, the stroke is a brain stem stroke. In another alternative of this embodiment, the stroke is a cerebellar stroke. In still another embodiment, the stroke is an embolic stroke. In yet another alternative, the stroke may be a hemorrhagic stroke. In a further embodiment, the stroke is a thrombotic stroke.

In yet another aspect, a sirtuin-modulating compound may be administered to reduce infarct size of the ischemic core following a central nervous system ischemic condition. Moreover, a sirtuin-modulating compound may also be beneficially administered to reduce the size of the ischemic penumbra or transitional zone following a central nervous system ischemic condition.

The use of HDAC inhibitors, including sirtuin inhibitors, to reprogram cells to generate pluripotent cells, e.g., for use in regenerative medicine has been described (WO 2010/56831).

In certain embodiments, a combination drug regimen may include drugs or compounds for the treatment or prevention of neurodegenerative disorders or secondary conditions associated with these conditions. Thus, a combination drug regimen may include one or more sirtuin activators and one or more anti-neurodegeneration agents.

### Inflammatory Diseases

In other aspects, sirtuin-modulating compounds that decrease the level and/or activity of a sirtuin protein can be used to treat or prevent a disease or disorder associated with inflammation. Sirtuin-modulating compounds that decrease the level and/or activity of a sirtuin protein may be administered prior to the onset of, at, or after the initiation of inflammation. When used prophylactically, the compounds are preferably provided in advance of any inflammatory response or symptom. Administration of the compounds may prevent or attenuate inflammatory responses or symptoms.

In another embodiment, sirtuin-modulating compounds that decrease the level and/or activity of a sirtuin protein may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The compounds may be used to treat chronic hepatitis infection, including hepatitis B and hepatitis C.

Additionally, sirtuin-modulating compounds that decrease the level and/or activity of a sirtuin protein may be used to treat autoimmune diseases, and/or inflammation associated with autoimmune diseases, such as arthritis, including rheumatoid arthritis, psoriatic arthritis, and ankylosing spondylitis, as well as organ-tissue autoimmune diseases (e.g., Raynaud's syndrome), ulcerative colitis, Crohn's disease, oral mucositis, scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

In certain embodiments, one or more sirtuin-modulating compounds that decrease the level and/or activity of a sirtuin protein may be taken alone or in combination with other compounds useful for treating or preventing inflammation.

### 4. Assays

Yet other methods contemplated herein include screening methods for identifying compounds or agents that modulate sirtuins. An agent may be a nucleic acid, such as an aptamer. Assays may be conducted in a cell based or cell free format. For example, an assay may comprise incubating (or contacting) a sirtuin with a test agent under conditions in which a sirtuin can be modulated by an agent known to modulate the sirtuin, and monitoring or determining the level of modulation of the sirtuin in the presence of the test agent relative to the absence of the test agent. The level of modulation of a sirtuin can be determined by determining its ability to deacetylate a substrate. Other substrates are peptides from human histones H3 and H4 or an acetylated amino acid. Substrates may be fluorogenic. The sirtuin may be SIRT1, SIRT2, SIRT3, or a portion thereof. The level of modulation of the sirtuin in an assay may be compared to the level of modulation of the sirtuin in the presence of one or more (separately or simultaneously) compounds described herein, which may serve as positive or negative controls.

In certain embodiments, the deacetylation of a Trp 5-mer peptide (Ac-RHKK_{Ac}W-NH, Biopeptide, San Diego, Calif.) by His-SIRT1(1-747), His-SIRT2(1-389) and His-SIRT3(102-399) was measured by a discontinuous OAADPr Mass Spec assay which measures OAADPr (2′-O-acetyl-ADP-ribose) production. All assays were performed at room temperature in reaction buffer (50 mM HEPES, pH 7.5, 150 mM NaCl, 1 mM DTT, 0.05% BSA). Test compounds (1 μL in DMSO) were pre-incubated with either SIRT1 (5 nM), SIRT2 (10 nM) or SIRT3 (5 nM) in reaction buffer (50 μL) for 20 minutes. For IC₅₀ determination, Trp 5-mer peptide was added at K_{M} conditions (2 μM for SIRT1, 10 μM for SIRT2 or 2.2 μM for SIRT3) along with NAD at K_{M} (80 μM for SIRT1, 50 μM for SIRT2 and 130 μM SIRT3) for a final volume of 100 μL. The reaction was quenched after 30 minutes with 10 μL of stop buffer (50 mM nicotinamide in 10% formic acid) to give a final concentration of 0.9% formic acid and 4.5 mM nicotinamide. To prepare the assays for analysis, 20 μL of reaction volume was mixed in 80 μL of 50:50 acetonitrile:methanol mixture. The plates were analyzed on an Agilent RapidFire 200 High-Throughput Mass Spectrometry System (Agilent, Wakefield) coupled to an AB Sciex API 4000 mass spectrometer fitted with an electrospray ionization source in negative MRM mode monitoring the transition 600.1/345.9 for the parent/daughter ion under low resolution conditions. Peak data was integrated using RapidFire Integrator software (Agilent, Santa Clara, Calif.).

### 5. Pharmaceutical Compositions

The compounds described herein may be formulated in a conventional manner using one or more physiologically or pharmaceutically acceptable carriers or excipients. For example, compounds and their pharmaceutically acceptable salts and solvates may be formulated for administration by, for example, injection (e.g. SubQ, IM, IP), inhalation or insufflation (either through the mouth or the nose) or oral, buccal, sublingual, transdermal, nasal, parenteral or rectal administration. In certain embodiments, a compound may be administered locally, at the site where the target cells are present, i.e., in a specific tissue, organ, or fluid (e.g., blood, cerebrospinal fluid, etc.).

The compounds can be formulated for a variety of modes of administration, including systemic and topical or localized administration. Techniques and formulations generally may be found in Remington's Pharmaceutical Sciences, Meade Publishing Co., Easton, Pa. For parenteral administration, injection is preferred, including intramuscular, intravenous, intraperitoneal, and subcutaneous. For injection, the compounds can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets, lozenges, or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For administration by inhalation (e.g., pulmonary delivery), the compounds may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, compounds may also be formulated as a depot preparation. Such long acting formulations may be administered by implantation (for example subcutaneously or intramuscularly) or by intramuscular injection. Thus, for example, compounds may be formulated with suitable polymeric or hydrophobic materials (for example as an emulsion in an acceptable oil) or ion exchange resins, or as sparingly soluble derivatives, for example, as a sparingly soluble salt. Controlled release formula also includes patches.

In certain embodiments, the compounds described herein can be formulated for delivery to the central nervous system (CNS) (reviewed in Begley, et al. (2004) Pharmacology & Therapeutics 104, 29-45). Conventional approaches for drug delivery to the CNS include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide).

Liposomes are a further drug delivery system which is easily injectable. Accordingly, in the method of invention the active compounds can also be administered in the form of a liposome delivery system. Liposomes are well known by those skilled in the art. Liposomes can be formed from a variety of phospholipids, such as cholesterol, stearylamine of phosphatidylcholines. Liposomes usable for the method of invention encompass all types of liposomes including, but not limited to, small unilamellar vesicles, large unilamellar vesicles and multilamellar vesicles.

Another way to produce a formulation, particularly a solution, of a compound described herein, is through the use of cyclodextrin. By cyclodextrin is meant α-, β-, or γ-cyclodextrin. Cyclodextrins are described in detail in Pitha et al., U.S. Pat. No. 4,727,064, which is incorporated herein by reference. Cyclodextrins are cyclic oligomers of glucose; these compounds form inclusion complexes with any drug whose molecule can fit into the lipophile-seeking cavities of the cyclodextrin molecule.

Rapidly disintegrating or dissolving dosage forms are useful for the rapid absorption, particularly buccal and sublingual absorption, of pharmaceutically active agents. Fast melt dosage forms are beneficial to patients, such as aged and pediatric patients, who have difficulty in swallowing typical solid dosage forms, such as caplets and tablets. Additionally, fast melt dosage forms circumvent drawbacks associated with, for example, chewable dosage forms, wherein the length of time an active agent remains in a patient's mouth plays an important role in determining the amount of taste masking and the extent to which a patient may object to throat grittiness of the active agent.

Pharmaceutical compositions (including cosmetic preparations) may comprise from about 0.00001 to 100% such as from 0.001 to 10% or from 0.1% to 5% by weight of one or more compounds described herein. In other embodiments, the pharmaceutical composition comprises: (i) 0.05 to 1000 mg of the compounds of the invention, or a pharmaceutically acceptable salt thereof, and (ii) 0.1 to 2 grams of one or more pharmaceutically acceptable excipients.

In some embodiments, a compound described herein is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Formulations may be colorless, odorless ointments, lotions, creams, microemulsions and gels.

The compounds may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing.

The compounds may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type.

The compounds may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

The compounds may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9).

The compounds may be incorporated into gel formulations, which generally are semisolid systems consisting of either suspensions made up of small inorganic particles (two-phase systems) or large organic molecules distributed substantially uniformly throughout a carrier liquid (single phase gels). Although gels commonly employ aqueous carrier liquid, alcohols and oils can be used as the carrier liquid as well.

Additional active agents may also be included in formulations, e.g., other anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Conditions of the eye can be treated or prevented by, e.g., systemic, topical, intraocular injection of a compound, or by insertion of a sustained release device that releases a compound. A compound may be delivered in a pharmaceutically acceptable ophthalmic vehicle, such that the compound is maintained in contact with the ocular surface for a sufficient time period to allow the compound to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically acceptable ophthalmic vehicle may, for example, be an ointment, vegetable oil or an encapsulating material. Alternatively, the compounds of the invention may be injected directly into the vitreous and aqueous humour. In a further alternative, the compounds may be administered systemically, such as by intravenous infusion or injection, for treatment of the eye.

The compounds described herein may be stored in oxygen free environment. For example, a composition can be prepared in an airtight capsule for oral administration, such as Capsugel from Pfizer, Inc.

Cells, e.g., treated ex vivo with a compound as described herein, can be administered according to methods for administering a graft to a subject, which may be accompanied, e.g., by administration of an immunosuppressant drug, e.g., cyclosporin A. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000.

Toxicity and therapeutic efficacy of compounds can be determined by standard pharmaceutical procedures in cell cultures or experimental animals. The LD₅₀ is the dose lethal to 50% of the population. The ED₅₀ is the dose therapeutically effective in 50% of the population. The dose ratio between toxic and therapeutic effects (LD₅₀/ED₅₀) is the therapeutic index. Compounds that exhibit large therapeutic indexes are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may lie within a range of circulating concentrations that include the ED50 with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### 6. Kits

Also provided herein are kits, e.g., kits for therapeutic purposes or kits for modulating the lifespan of cells or modulating apoptosis. A kit may comprise one or more compounds as described herein, e.g., in premeasured doses. A kit may optionally comprise devices for contacting cells with the compounds and instructions for use. Devices include syringes, stents and other devices for introducing a compound into a subject (e.g., the blood vessel of a subject) or applying it to the skin of a subject.

In yet another embodiment, the invention provides a composition of matter comprising a compound of this invention and another therapeutic agent (the same ones used in combination therapies and combination compositions) in separate dosage forms, but associated with one another. The term “associated with one another” as used herein means that the separate dosage forms are packaged together or otherwise attached to one another such that it is readily apparent that the separate dosage forms are intended to be sold and administered as part of the same regimen. The compound and the other agent are preferably packaged together in a blister pack or other multi-chamber package, or as connected, separately sealed containers (such as foil pouches or the like) that can be separated by the user (e.g., by tearing on score lines between the two containers).

In still another embodiment, the invention provides a kit comprising in separate vessels, a) a compound of this invention; and b) another therapeutic agent such as those described elsewhere in the specification.

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N. Y., 1986).

### EXEMPLIFICATION

The invention now being generally described, it will be more readily understood by reference to the following examples which are included merely for purposes of illustration of certain aspects and embodiments of the present invention, and are not intended to limit the invention in any way.

### Example 1

### Encoded Library Technology (ELT) Screen

An in vitro SIRT3 affinity selection from an Encoded Library Technology (ELT) screen (Clark, M. A. et al. (2009) Nat Chem Biol 5, 647-654 and Deng, H. et al. (2012) J Med Chem 55, 7061-7079) was utilized to identify the initial SIRT1/2/3 pan inhibitors. ELT is a robust hit identification platform that employs large collections of chemotypically diverse DNA-encoded small molecule libraries which are screened for their affinity towards a desired protein target. The technology provides access to a broad set of chemotypes with structural diversity in an evolving library collection. It is also an attractive platform because it uses negligible amounts of target protein to carry out selection experiments, and it identifies ligands regardless of their functional activity. ELT has been successfully used to identify hits against a number of soluble targets over the past few years (Evindar, G. et al. (2009) 238th National Meeting of the American Chemical Society, Washington, D.C., August 16-20, pp MEDI-126; Graybill, T. L. (2009) 237th National Meeting of the American Chemical Society, Salt Lake City, Utah, March 22-26, pp MEDI-297; Davie, C. P. et al. (2010) 240th National Meeting of the American Chemical Society, Boston, Mass., United States, August 22-26, pp MEDI-150; Ding, Y. et al. (2010) 240th National Meeting of the American Chemical Society, Boston, Mass., United States, August 22-26, pp MEDI-150; and Gentile, G. et al. (2012) Bioorg Med Chem Lett 22, 1989-1994).

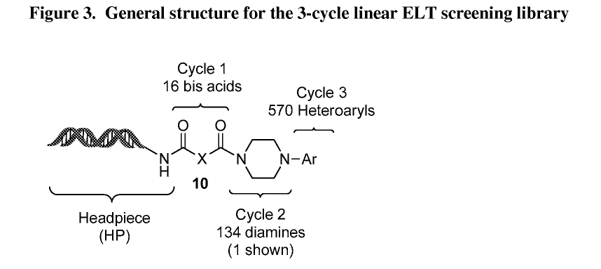

An ELT selection campaign was carried out against a Flag-SBP-tagged SIRT3 construct. Flag-SIRT3-SBP was immobilized on streptavidin matrix tips, and selections were performed under three different conditions: SIRT3 alone; SIRT3 plus β-Nicotinamide Adenine Dinucleotide (NAD⁺); and SIRT3 plus thioacetyl-peptide AceCS2 substrate (TRSGK_{s-Ac}VMRRLLR) (Jin, L. et al. (2009) J Biol Chem 284, 24394-24405). The SIRT3 selection conditions were used to screen a 3-cycle linear library capped with heteroaryl moieties (Compound 10, FIG. 3). The library was established by coupling 16 bis-acid building blocks (cycle 1) to the ELT headpiece (HP) which allowed for further elaboration of the second carboxylate with 134 diamines (cycle 2). The second amine from cycle 2 was then functionalized with 570 heteroaryl building blocks (cycle 3) to afford an ELT library with 1.2 million enumerated compounds.

ELT affinity selections were carried out by capturing 2 μg of Flag-hSIRT3(118-399)-SBP on streptavidin matrix tips (Phynexus) in the presence of 1) no β-NAD/no peptide substrate, 2) 100 μM β-NAD (Sigma), or 3) 20 μM TRSGK_{thioacetyl}VMRRLLR for three rounds. A no target control selection with buffer was carried out concurrently in the absence of SIRT3 protein. Streptavidin tips were pre-washed in selection buffer: 50 mM Tris (pH 7.5), 150 mM NaCl, 0.1% Tween-20, and 0.1 mg/mL sheared salmon sperm DNA (sssDNA, Ambion), 0.1 mg/mL BSA (Ambion) and 5 mM β-mercaptoethanol (BME). In the first round of selection, 2 μg of Flag-hSIRT3(118-399)-SBP protein was immobilized on pre-washed tips in the presence of 1) no β-NAD/no peptide substrate, 2) 100 μM β-NAD or 3) 20 μM thioacetylated peptide. The tips were washed two times with buffer containing the corresponding cofactor and substrate when necessary. Pooled ELT libraries (5 nmoles) were passed over the immobilized SIRT3 in the presence of the corresponding cofactor and substrate for 1 hour at room temperature. The tips were washed 8 times with selection buffer containing the corresponding cofactor and substrate and two times with BSA free selection buffer containing the corresponding cofactor and substrate. Bound molecules were heat eluted by passing BSA free selection buffer containing no cofactors and substrates over the tip at 72° C. for 10 minutes. The cooled heat elution was post-cleared twice by passing the elution over streptavidin tips for 15 min to remove any denatured SIRT3 and matrix binders. Fresh BSA and sssDNA were added to all samples and corresponding cofactor and substrate was added to the elutions as needed. Round 2 was performed as described for Round 1 using freshly immobilized SIRT3 on streptavidin tips in the presence of corresponding cofactor and substrate and post-cleared Round 1 output. Round 3 was performed as described for Round 1 using freshly immobilized SIRT3 on streptavidin tips in the presence of corresponding cofactor and substrate and post-cleared Round 2 output with the exceptions that the last two washes and elution were with BSA-free and ssDNA-free selection buffer and the round 3 output was not post-cleared. Quantitative PCR was used to quantitate the outputs from each round of selection. The round 3 output was sequenced using an Illumina sequencing platform.

Human SIRT3-(118-399) was cloned into a modified pET21b vector (Novagen). The protein was expressed in E. coli BL21-Gold(DE3) cells (Stratagene) as an N-terminal fusion to a hexahistidine affinity tag with an integrated TEV protease site. A single colony was inoculated in LB media containing 100 μg/ml ampicillin at 37° C., swirled at 250 rpm until the A₆₀₀ reached 0.3. The culture was then cooled to 18° C., swirled at 250 rpm until the A₆₀₀ reached 0.6-0.8. 1-(2-Isopropylthio)-β-D-galactopyranoside (IPGT) was added to a final concentration of 0.3 mM, and expression was continued at 18° C., swirled at 160 rpm overnight. Cells were collected by centrifugation, and the pellet was re-suspended in lysis buffer (200 mM NaCl, 5% glycerol, 5 mM 2-mercaptoethanol, and 25 mM HEPES-NaOH, pH 7.5) and sonicated to break the cells. The supernatant was separated from the cell debris by centrifugation at 10,000×g for 40 min at 4° C. and loaded onto a Ni-NTA column (Qiagen) that was equilibrated with a buffer containing 200 mM NaCl, 5% glycerol, 5 mM 2-mercaptoethanol, 20 mM imidazole, and 25 mM HEPES-NaOH, pH 7.5. The column was washed with 5 column volumes of a buffer containing 200 mM NaCl, 5% glycerol, 5 mM 2-mercaptoethanol, 50 mM imidazole, and 25 mM HEPES-NaOH, pH 7.5, then eluted with a buffer containing 200 mM NaCl, 5% glycerol, 5 mM 2-mercaptoethanol, 250 mM imidazole, and 25 mM HEPES-NaOH, pH 7.5. The eluted protein was dialyzed in lysis buffer and digested with TEV protease (Invitrogen) at 4° C. overnight to remove the N-terminal His tag. The protein was loaded on a second Ni-NTA column equilibrated with lysis buffer. The untagged protein was eluted with a buffer containing 200 mM NaCl, 5% glycerol, 5 mM 2-mercaptoethanol, 5 mM imidazole, and 25 mM HEPES-NaOH, pH 7.5. The purified protein was dialyzed against a buffer containing 200 mM NaCl, 5 mM 2-mercaptoethanol, and 20 mM Tris-HCl, pH 8.0, and concentrated. The protein was further purified by elution with dialyzing buffer over a S200 column (GE Healthcare) to 95% purity as assessed by SDS-PAGE analysis stained by Coomassie Brilliant Blue R-250, and concentrated to 10-15 mg/ml in the dialyzing buffer.

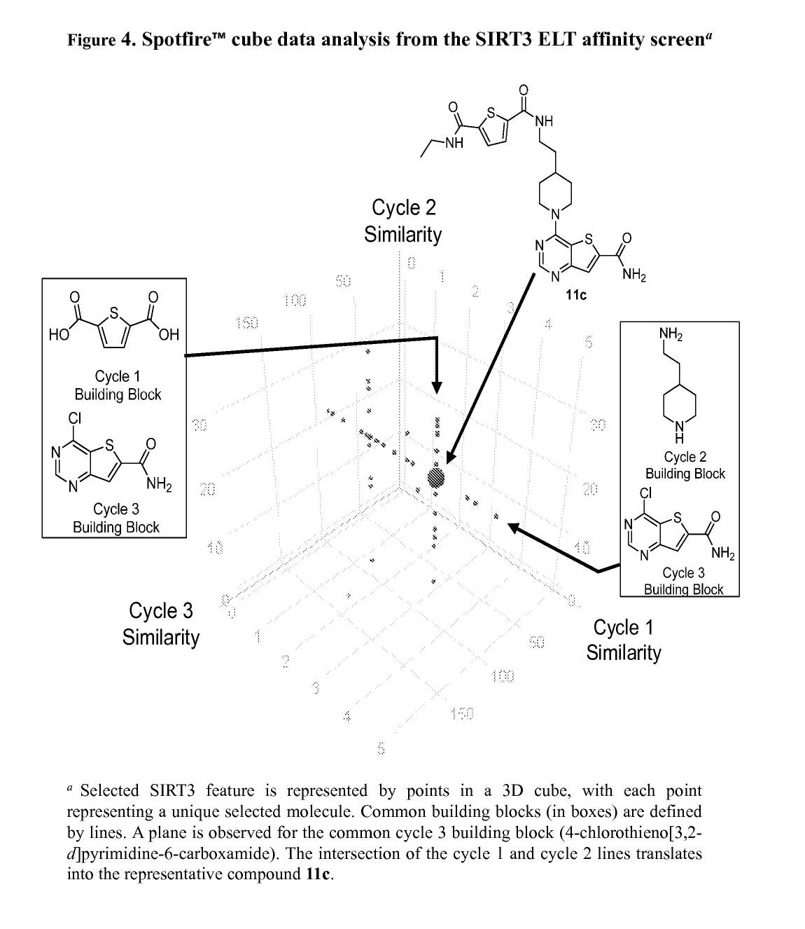

The sequencing data obtained from the ELT screen was transferred into a cubic scatter plot for visualization and analysis within Spotfire™, where each axis represents a cycle of diversity in the library (see FIG. 4). The background noise, single hits, and low copy number molecules were removed to simplify the data analysis and allow for closer observation of the more highly enriched families and features within the cube.

The primary chemotype was represented by a horizontal and a vertical line intersecting at a single point in the cube. These lines define a plane in cycle 3 originated from the 4-chlorothieno[3,2-d]pyrimidine-6-carboxamide building block connected to cycle 2 through an amine displacement of the chloride. The horizontal and vertical lines selected within the plane originated from combination of the selected cycle 3 building block and a specific cycle 1 or cycle 2 building block, thiophene-2,5-dicarboxylic acid and 2-(piperidin-4-yl)ethanamine, respectively.

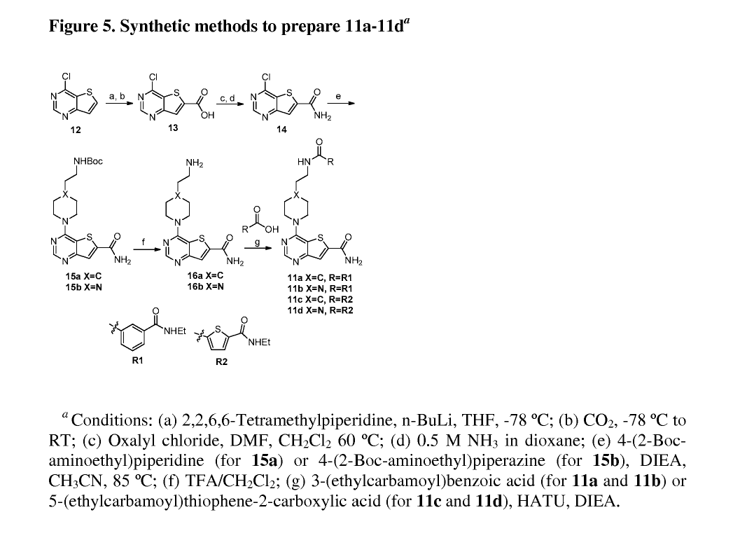

Due to the greater variety of cycle 1 and cycle 2 building blocks, depicted as the two blue lines, the pharmacophore most frequently observed is represented by the intersection product (Compound 11c) represented as the large dot. For simplicity the attachment point to DNA has been substituted by an ethylamide. Given the larger variability observed for the selection output of both cycle 1 and cycle 2 residues, depending on which line is analyzed, an additional cycle 1 and cycle 2 building blocks (isophthalic acid and 2-(piperazin-1-yl)ethanamine) were selected and a simple 2×2 library was synthesized to confirm off-DNA biochemical activity (see FIG. 5). This produced a sufficient number of off-DNA compounds to confirm activity of the chemotype and allowed for potential off-DNA preliminary SAR studies.

A novel class of potent SIRT1/2/3 pan inhibitors was identified by utilizing encoded library technology to enrich for molecules that interact with SIRT3 from a collection of diverse ELT libraries. Based on the analysis of the ELT sequencing data, SAR studies were carried out and revealed that the selected cycle 3 thieno[3,2-d]pyrimidine-6-carboxamide was the core scaffold and critical for the chemotype inhibitory function, and cycle 1 and cycle 2 could be more variable.

### Example 2

### Synthesis of 2×2 Library Structures

Assembly of the 2×2 library structures from the selected chemotype was accomplished by carboxylating commercially available 4-chlorothieno[3,2-d]pyrimidine (Compound 12, see FIG. 5) to obtain carboxylic acid Compound 13. Treating Compound 13 with oxalyl chloride, and quenching the intermediate acid chloride with ammonia in dioxane afforded the versatile 4-chlorothieno[3,2-d]pyrimidine-6-carboxamide intermediate Compound 14. The chloride on Compound 14 was subsequently displaced with 4-(2-Boc-aminoethyl)piperidine or 4-(2-Boc-aminoethyl)piperazine to afford the Boc-protected precursors (Compounds 15a and 15b). The Boc groups were removed by treatment with trifluoroacetic acid in CH₂Cl₂, and the resulting amines (Compounds 16a and 16b) were reacted under standard amide coupling conditions (HATU, DIEA) with (3-(ethylcarbamoyl)benzoic acid or 5-(ethylcarbamoyl)thiophene-2-carboxylic acid to afford Compounds 11a-d. The majority of the other disclosed analogs were synthesized by a similar route (i.e. substitution, followed by deprotection and amide coupling).

### Example 3

### Evaluation of Representative Off-DNA Compounds in a SIRT1/2/3 Biochemical Assay

The four off-DNA synthesized representative compounds (see Table 1, Compounds 11a-d) were evaluated for their ability to inhibit the sirtuin mediated deacetylation of a minimal peptide substrate (Ac-RHKK^{Ac}W-NH₂) (Dai, H. et al. (2010) J Biol Chem 285, 32695-32703) in a biochemical assay with SIRT1, SIRT2 and SIRT3 (see detailed description for specific assay conditions). The activity data was evaluated, along with physicochemical and calculated properties (kinetic solubility, CHI LogD (Stepanic, V. et al. (2012) Eur J Med Chem 47, 462-472), tPSA, CLogP) to determine drug-like properties.

TABLE 1
Sirtuin inhibition activity of off-tag ELT screening hits

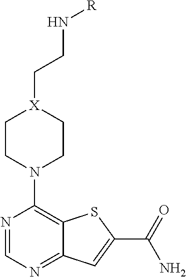

| | | | IC₅₀ (μM)ⁱ | | | | Solubilityⁱⁱⁱ | | | |
|---|---|---|---|---|---|---|---|---|---|---|
| Cmpd | R | X | SIRT1 | SIRT2 | SIRT3 | LogDⁱⁱ | (μM) | MW | CLogP | tPSA |
| 11a 11b | 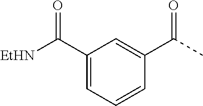 | CH N | 0.014 0.067 | 0.005 0.010 | 0.001 0.024 | 2.09 1.45 | 19 220 | 481 482 | 2.16 1.74 | 129 132 |
| 11c 11d | 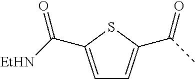 | CH N | 0.004 0.031 | 0.003 0.005 | 0.004 0.029 | 2.09 1.32 | 9 65 | 487 488 | 1.92 1.50 | 129 132 |

ⁱIC₅₀ values were determined from three separate titration curves. Each of the IC₅₀ values shown represents the mean of at least three determinations, with variation in individual values of <50%.
ⁱⁱLogD was determined by a HPLC based lipophilicity assay³⁴ by measuring Chromatographic Hydrophobicity Index (CHI) values by reverse phase HPLC and transforming them to a LogD scale based on known standards.
ⁱⁱⁱKinetic solubility was determined by a Chemi-luminescent nitrogen detection (CLND) solubility assay.³⁴ DMSO stock solutions were incubated (1 hr) in phosphate buffered saline (pH 7.4), filtered and measured by CLND.

The representative common chemotype Compound 11c, displayed excellent SIRT3 potency with an IC₅₀ of 4 nM and confirmed that the off-DNA chemotype was a functional inhibitor for SIRT3, and not merely a ligand with strong affinity. It was also observed to have analogous potency against SIRT1 and SIRT2. Collectively all the compounds in the 2×2 library (Compounds 11a-d) were very potent pan inhibitors of SIRT1, SIRT2 and SIRT3. Replacement of the piperidine of Compound 11c with a piperazine (Compound 11d) only slightly reduced the potency against SIRT2 (≦2-fold) while reducing inhibition of SIRT1 and SIRT3 about 7-8 fold. The corresponding phenyl based analogs (Compounds 11a and 11b) showed a similar trend. Comparing the effect of replacing the thiophene of Compounds 11c and 11d with phenyl (Compounds 11a and 11b) revealed a general decrease in potency for SIRT1 and SIRT2 (2-3 fold), whereas an improvement in SIRT3 activity (Compound 11a, SIRT3 IC₅₀=1 nM) was observed. Further analysis of Compounds 11a-d, revealed that the more basic piperazines imparted a lowering of CLogP, LogD and a concurrent 10-fold improvement in aqueous solubility. The change from thiophene to benzene had little effect on physiochemical properties.

The off-DNA compounds, Compounds 11a, 11b, 11c and 11d, represent a novel and highly potent class of SIRT1/2/3 pan inhibitors. While the goal of identifying novel sirtuin inhibitor scaffolds was achieved, inhibitors Compounds 11a-d tended to have suboptimal drug-like properties (MW, PSA, solubility, aromatic ring count) which limited their progression.

### Example 4

### Structure Activity Relationship (SAR) Studies

To explore the possibility of improving the physiochemical properties of Compound 11c, the most potent analog, while maintaining biochemical potency the pyrimidylthiophene carboxamide core was maintained and was systematically truncated from the DNA tag end of Compound 11c. A series of truncated analogs (see Table 2) were prepared and evaluated in the SIRT1, SIRT2 and SIRT3 biochemical inhibition assays (see detailed description for specific assay conditions).

TABLE 2
SIRT 1/2/3 inhibition of the truncated analogs of 11c

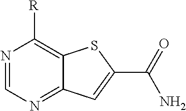

| | | IC₅₀ (μM)ⁱ | | |
|---|---|---|---|---|
| Cmpd | R | SIRT1 | SIRT2 | SIRT3 |
| 11c | 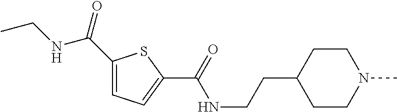 | 0.004 | 0.003 | 0.004 |
| 17 | 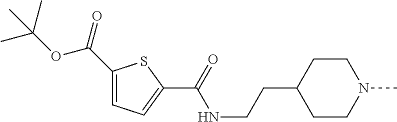 | 0.007 | 0.001 | 0.003 |
| 18 | 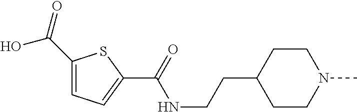 | 0.014 | 0.004 | 0.013 |
| 19 | 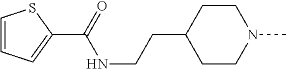 | 0.006 | 0.007 | 0.008 |
| 15a | 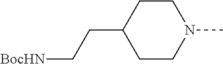 | 0.017 | 0.005 | 0.029 |
| 20 | 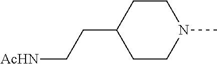 | 0.110 | 0.023 | 0.056 |
| 16a | 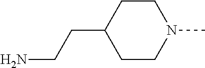 | 1.6 | 0.11 | 0.30 |
| 21 | 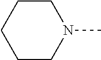 | 15 | 1.4 | 8.6 |
| 22 | EtNH— | >50 | 49 | >50 |
| 14 | Cl— | >50 | >50 | >50 |

ⁱIC₅₀ values were determined from three separate titration curves. Each of the IC₅₀ values shown represent the mean of at least three determinations, with variation in individual values of <50%.

Changing the ethylamide substituent on the thiophene to either a tert-butyl ester (Compound 17, see Table 2), carboxylic acid Compound 18 or hydrogen Compound 19 resulted in modest (ca. 2 fold) reduction of SIRT1, SIRT2 or SIRT3 inhibition compared to Compound 11c, suggesting that the terminal ethylamide is not essential for activity.

Due to these above results, the impact of removing the thiophene ring was evaluated. Replacing the thiophene in Compound 19, with a t-butyl carbamate (Compound 15a) resulted in a 3 to 4 fold reduction of SIRT1 and SIRT3 activity, while SIRT2 inhibition remained unchanged. When the thiophene in Compound 19 was exchanged for an acetamide (Compound 20), we observed more significant reductions in SIRT1 (18 fold), SIRT2 (3 fold) and SIRT3 (7 fold) activity. This trend continued with the removal of the acetamide in the amine analog Compound 16a (SIRT1, SIRT2 and SIRT3 were 15, 5 and 5 fold less potent than Compound 20 respectively). Replacing the aminoethylpiperidine with a piperidine Compound 21 dramatically reduced the sirtuin inhibitory activity to micromolar levels. Lastly, SIRT1/2/3 inhibitory activity was not conserved in the severely truncated ethylamine Compound 22 or chloride Compound 14, indicating that the thieno[3,2-d]pyrimidine-6-carboxamide core alone was not sufficient to produce observeable SIRT1/2/3 inhibition.

Of the truncated analogs presented, Compound 20 exhibited the most balanced SIRT1/2/3 inhibitory activity (23-110 nM) and a low molecular weight (MW=347). To further optimize the potency, a larger compound set (see Table 3) was prepared wherein variation of three groups was explored: 1) the acetamide functionality on Compound 20 and its replacement with a thioacetyl-, pivaloyl-, sulfonamide or a pyrrolidine group, 2) the linker length (n) was varied to determine the optimal distance between the functional group and the thienopyrimidine core and 3) the effect of changing the piperidine to the more polar piperazine ring system (where X═CH or N) was explored.

TABLE 3
Effect of aliphatic functionality and linker length on SIRT 1/2/3 inhibition

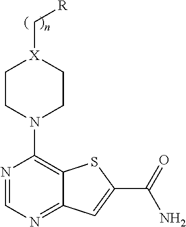

| | | | | IC₅₀ (μM)ⁱ | | |
|---|---|---|---|---|---|---|
| Cmpd | R | X | n | SIRT1 | SIRT2 | SIRT3 |
| 4, EX-527 | — | — | — | 0.26 | 2.9 | >50 |
| 23 | —NH(C═O)Me | CH | 1 | 16 | 2.8 | 4.4 |
| 20 | —NH(C═O)Me | CH | 2 | 0.11 | 0.023 | 0.056 |
| 24 | —NH(C═O)Me | N | 2 | 1.6 | 0.17 | 0.77 |
| 25 | —NH(C═S)Me | CH | 2 | 0.056 | 0.030 | 0.039 |
| 26 | —NH(C═S)Me | N | 2 | 0.49 | 0.13 | 0.28 |
| 27 | —NH(C═O)tBu | CH | 1 | 4.3 | 0.74 | 0.65 |
| 28 | —NH(C═O)tBu | CH | 2 | 0.015 | 0.010 | 0.033 |
| 29 | —NH(C═O)tBu | CH | 3 | 0.042 | 0.016 | 0.067 |
| 30 | —NH(C═O)tBu | N | 2 | 0.12 | 0.017 | 0.092 |
| 31 | —NHSO₂Me | CH | 2 | 0.004 | 0.001 | 0.007 |
| 32 | —NHSO₂Me | N | 2 | 0.38 | 0.053 | 0.37 |
| 33 | 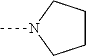 | CH | 1 | 0.73 | 0.22 | 0.061 |
| 34 | 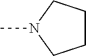 | CH | 2 | 0.11 | 0.067 | 0.028 |
| 35 | 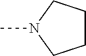 | N | 2 | 0.053 | 0.029 | 0.030 |

ⁱIC₅₀ values were determined from three separate titration curves. Each of the IC₅₀ values shown represents the mean of at least three determinations, with variation in individual values of <50%.

In general, the ethyl piperidines (where X═CH and n=2) were more potent inhibitors than the ethyl piperazines (where X═N and n=2), (compare Compounds 20 vs. 24, 25 vs. 26, 28 vs. 30, 31 vs. 32). However, for the pyrrolidine analogs (Compounds 34 and 35) the piperazine (where X═N) analog displayed similar or greater potency on SIRT1, SIRT2 or SIRT3 than did the corresponding piperidine (where X═CH) analog.

Linker length changes were evaluated on the acetamides (Compounds 20 and 23), pivaloylamides (Compounds 27-29), and the pyrrolidines (Compounds 33 and 34). Taking the case of the pivaloylamides, the ethyl linker (Compound 28, where n=2) is slightly more potent than the longer propyl linker (Compound 29, where n=3). The shorter methylene linker (Compound 27, where n=1) led to a dramatic decrease in SIRT1, SIRT2 and SIRT3 potency. This dramatic decrease in potency with shorter (where n=1) linker length was broadly observed in other analogs (Compounds 23 and 33).

Replacing the acetamide (Compounds 20 and 24) with a thioamide (Compounds 25 and 26) was well tolerated, resulting in modest improvements in potency. Changing the acetyl groups to the more lipophilic pivaloylamide or the polar sulfonamides was advantageous for sirtuin inhibition. Interestingly, the sulfonamide Compound 31 which contains the optimal structural elements for inhibition (where X═CH and n=2) is a single digit nanomolar inhibitor of SIRT1, SIRT2 and SIRT3 and represents one of the most potent pan-inhibitors in the study. While evaluating the pyrrolidine analogs (Compound 33-35) it was observed that the selectivity profile appears to slightly favor SIRT3 inhibition over SIRT1 and SIRT2. In general, there appears to be broad functional group tolerance in this region of the inhibitors (e.g. lipophilic, polar, basic, H-bond donor or H-bond acceptor groups).

The SAR of the heteroaromatic thieno[3,2-d]pyrimidine core was also evaluated. Utilizing the potent pan inhibitor Compound 28 as a comparator, a small series of heteroaromatic carboxamide cores were prepared and their ability to inhibit SIRT1/2/3 (see Table 4) was evaluated. Replacing the core of Compound 28 (where X═S) with furo[3,2-d]pyrimidine-6-carboxamide (Compound 36, where X═O) resulted in a 15-40 fold reduction in SIRT1/2/3 potency. To evaluate the pyrimidine portion of the heteroaromatic core, two thienopyridine carboxamide scaffolds were prepared. The first thienopyridine analog, where N3 was replaced with a CH (Compound 37, where Y═CH and Z═N), resulted in a modest reduction in SIRT1/2/3 inhibition (3 to 4 fold). Whereas the replacement of N1 with CH (Compound 38, where Y═N and Z═CH) resulted in more significant reductions in potency (30-63 fold). To assess the sensitivity of the unsubstituted position of the thiophene ring, a methyl was added at the 7-position (Compound 39), which reduced activity by 4-12 fold across all three enzymes.

TABLE 4
Effect of modification of the thieno[3,2-d]pyrimidine core on
SIRT1/2/3 inhibition

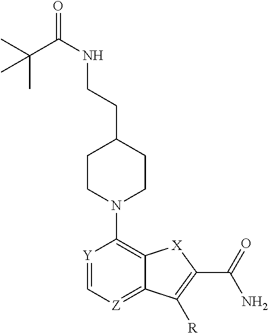

| | | | | | IC₅₀ (μM)ⁱ | | |
|---|---|---|---|---|---|---|---|
| Cmpd | X | Y | Z | R | SIRT1 | SIRT2 | SIRT3 |
| 28 | S | N | N | H | 0.015 | 0.010 | 0.033 |
| 36 | O | N | N | H | 0.47 | 0.15 | 1.3 |
| 37 | S | CH | N | H | 0.059 | 0.028 | 0.11 |
| 38 | S | N | CH | H | 0.49 | 0.30 | 2.1 |
| 39 | S | N | N | Me | 0.18 | 0.13 | 0.15 |

ⁱIC₅₀ values, expressed in μM, were determined from three separate titration curves. Each of the IC₅₀ values shown represents the mean of at least three determinations, with variation in individual values of <50%.

Lastly, the SAR of the thieno[3,2-d]pyrimidine carboxamide was evaluated (see Table 5) by making several small adjustments at the 6-position of the thieno[3,2-d]pyrimidine of (Compound 28, where R═(C═O)NH₂). The mono methylated amide (Compound 41, where R═—(C═O)NHCH₃) displayed a dramatic loss of SIRT2 activity (10 μM), and no measurable SIRT1 or SIRT3 activity. Similarly, changing the carboxamide to a carboxylic acid Compound 40 (where R═COOH) or complete removal of the carboxamide Compound 42 (where R═H) resulted in no measurable SIRT1, SIRT2 or SIRT3 inhibition at the concentrations tested. These results indicate that the carboxamide is important for maintaining SIRT1/2/3 inhibition, and it is likely involved in critical contacts with the protein. The sensitive nature of modifying the carboxamide is similar to SAR observed for carboxamide in EX-527 (Compound 4, Napper, A. D. et al. (2005) 48, 8045-8054).

TABLE 5
Effect of modification of the carboxamide on SIRT1/2/3 inhibition

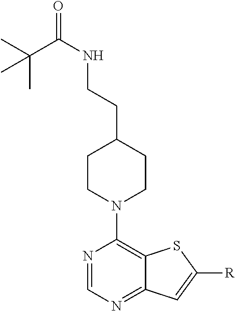

| | | IC50 (μM)ⁱ | | |
|---|---|---|---|---|
| Cmpd | R | SIRT1 | SIRT2 | SIRT3 |
| 28 | —(C═O)NH₂ | 0.015 | 0.010 | 0.033 |
| 40 | —(C═O)OH | >50 | >50 | >50 |
| 41 | —(C═O)NHMe | >50 | 10 | >50 |
| 42 | —H | >50 | >50 | >50 |

ⁱIC₅₀ values were determined from three separate titration curves. Each of the IC₅₀ values shown represents the mean of at least three determinations, with variation in individual values of <50%.

Reduction of molecular weight, to improve physiochemical properties of 11c, resulted in the identification of the acetamide Compound 20 as a good compromise of potency and reduced molecular weight. Further SAR identified that the thioacetyl (Compound 25), the tert-butyl-amide (Compound 28) and the sulfonamide (Compound 31) were particularly potent pan inhibitors. The low molecular weight (MW=383) and single digit nanomolar potency of Compound 31 makes it an exemplary compound.

### Example 5

### SIRT3 X-Ray Structural Studies

There have been several reported crystal structures for the sirtuins (Jin, L. et al. (2009) J Biol Chem 284, 24394-24405; Sanders, B. D. et al. (2010) 1804, 1604-1616; Szczepankiewicz, B. G. et al. (2012) 77, 7319-7329; Avalos, J. L. et al. (2005) 17, 855-868 and Finnin, M. S. et al. (2001) 8, 621-625), including SIRT2, SIRT3 and SIRT5. The sirtuins have variable N- and C-terminal regions, and a commonly conserved catalytic core which contains two lobes; a large Rossmann lobe, and a smaller lobe which contains a structural zinc binding motif. Acetylated substrates bind in a cleft formed at the interface of the two lobes with the acetylated lysine projecting toward the nicotinamide riboside portion of NAD⁺. A flexible loop, on the smaller lobe, closes down during the course of the deacetylation reaction to protect the imidate intermediate from solvent exposure.

Previously, we have reported the crystal structures of human SIRT3 (PDB code: 3GLS), substrate bound AceCS2/SIRT3 (PDB code: 3GLR), the imidate reaction intermediate mimetic SIRT3-AceCS2-K_{s-ac}-ADPR (PDB code: 3GLT) and the ternary carbaNAD/AceCS2/SIRT3 (PDB code: 4FVT). In this study, one of the identified ELT hits (Compound 11c), and the two most potent truncated pan SIRT1/2/3 inhibitors (Compounds 28 and 31) were evaluated by X-ray crystallography in complex with human SIRT3. Crystals of SIRT3 and the inhibitors were obtained by co-crystallization with SIRT3(118-399). Crystals of SIRT3/Compound 11c and SIRT3/Compound 31 diffracted to 1.70 and 2.25 Å, respectively. However, for SIRT3/Compound 28 the diffraction quality of the crystals was poor. Consequently Compound 28 had to be soaked/exchanged into SIRT3/Compound 31 crystals to achieve a suitable diffraction (2.26 Å).

Globally, the overall fold of the binary SIRT3/inhibitor structures is similar to that observed in previously reported structures of human SIRT3 (PDB codes: 3GLR, 3GLS, 3GLT, 3GLU, 4FVT). The Rossmann fold is highly superimposable, and there is a domain closure upon binding to substrates, cofactor intermediates (Jin, L. et al. (2009) J Biol Chem 284, 24394-24405 and Szczepankiewicz, B. G. et al. (2012) J Org Chem 77, 7319-7329) or active-site-targeting inhibitors (presented herein). The largest divergence among SIRT3 structures bound with different ligands occurs at the flexible loop I154-Y175, which closes down on the nicotinamide C-pocket.

To better understand their mode of action, three pan inhibitors (Compounds 11c, 28 and 31) were crystallized with SIRT3. The thieno[3,2-d]pyrimidine-6-carboxamide inhibitors bind in the active site cleft between the large Rossmann fold and the small zinc binding domain, occupying the nicotinamide C-pocket in addition to the substrate channel. Comparisons with other previously described SIRT3 structures reveal similar protein folding, except for the flexible loop region where F157 makes a π-stacking interaction with the thienopyrimidine core. The carboxamide of the inhibitors makes key hydrogen bonding interactions with the residues within the nicotinamide binding pocket, similar to carba-NAD. SAR studies corroborate that to maintain inhibitory activity the carboxamide group is required.

The SIRT3/Compound 31 and SIRT3/Compound 11c crystals were obtained by using a hanging drop vapor diffusion method at 18° C. The drop was comprised of a 1 μl protein/compound mixture and a 1 μl crystallization buffer. For SIRT3/31 the crystallization condition was 0.1 M HEPES pH 7.5 20% w/v PEG 8000. The crystallization buffer for SIRT3/11c was 0.1M Tris pH 8.0, 20% PEG 4000 or 20% PEG 6000. The SIRT3/Compound 31 and SIRT3/Compound 11c crystals were subsequently cryo-protected in the mother liquor, which contained 20% glycerol, prior to being flash-frozen in liquid nitrogen. The SIRT3/Compound 31 crystals soaked in Compound 28 were subsequently cryo-protected in the mother liquor, which contained 20% glycerol and 10 mM of Compound 28. The diffraction data was collected at Shanghai Synchrotron Radiation Facility (SSRF) beamline workstations BL17U1 and APS 21-ID-D and processed using Xia2 and HKL2000. The SIRT3 structures were solved by utilizing molecular replacement, using the substrate bound AceCS2/SIRT3 structure (PDB code: 3GLR) as a search model (see Table 6). In addition, all of the parameters for each diffraction data set were reprocessed using Mosflm and Scala and the refinement statistics were obtained from Refmac, a part of the CCP4 suite.

TABLE 6
Crystal Diffraction and Refinement Parameters

| Crystal | hSIRT3/11c | hSIRT3/28 | hSIRT3/31 |
|---|---|---|---|
| Diffraction Data | | | |
| Site | SSRF BL17U1 | APS 21-ID-D | SSRF BL17U1 |
| Data Processing | HKL2000 | Xia2 | HKL2000 |
| Program | 40.00-1.70 | 38.39-2.26 | 50.00-2.25 |
| Resolution (Å)* | (1.76-1.70) | (2.32-2.26) | (2.33-2.25) |
| Space group | P6₅ | P6₅ | P6₅ |
| Unit-cell parameters | | | |
| a (Å) | 119.32 | 117.28 | 120.46 |
| b (Å) | 119.32 | 117.28 | 120.46 |
| c (Å) | 44.53 | 45.59 | 44.47 |
| α (°) | 90.00 | 90.00 | 90.00 |
| β (°) | 90.00 | 90.00 | 90.00 |
| γ (°) | 120.00 | 120.00 | 120.00 |
| Completemess (%)* | 100.0 (100.0) | 99.7 (98.7) | 98.9 (100.0) |
| Redundancy* | 5.4 (5.4) | 9.1 (9.4) | 10.6 (10.4) |
| Average I/σI* | 29..3 (2.6) | 13.0 (4.1) | 25.6 (7.0) |
| R_{merge} (%)* | 7.9 (51.1) | 10.9 (103.9) | 9.8 (47.7) |
| Refinement Statistics | | | |
| Refinement Program | Refmac5 | Refmac5 | Refmac5 |
| | 103.72-1.80 | 38.39-2.26 | 39.43-2.24 |
| Data (no cutoff) | (1.85-1.80) | (2.32-2.26) | (2.30-2.24) |
| (Å)* | | | |
| R_{working} (%)* | 19.0 (29.8) | 20.2 (25.8) | 19.7 (41.3) |
| R_{free} (%)* | 23.7 (37.4) | 24.1 (34.5) | 24.7 (42.8) |
| R.M.S.D in bond | 0.03 | 0.01 | 0.011 |
| lengths (Å) | | | |
| R.M.S.D in bond | 2.334 | 1.242 | 1.27 |
| angles (°) | | | |
| Mean B factors (Å²) | 25.5 | 42.7 | 41.1 |

*Values in parentheses are for the highest-resolution shell

SRT1/2/3 inhibitor Compounds 11c, 28 and 31 bind identically to the catalytic active site (RMS=0.29 Å), occupying the acetyl lysine substrate channel and the nicotinamide C-pocket. During the course of binding, when the inhibitors are located in the catalytic site, the small lobe shifts slightly onto the larger Rossmann-fold, and the flexible loop (I154-Y175) closes down on the inhibitor. Closer evaluation of the binding interactions of Compounds 11c, 28 and 31 reveals that the arylcarboxamide makes four hydrogen bonds with the protein surface, similar to the analogous nicotinamide portion of carba-NAD⁺. The 6-carboxamide carbonyl of Compounds 11c, 28 and 31 accepts a hydrogen bond from the NH of I230 and D231, which are located on the protein backbone. The carboxamide NH of Compounds 11c, 28 and 31 forms a hydrogen bond with the lone pair of the carboxylic acid oxygen of D231, and the other carboxamide hydrogen of Compounds 11c, 28 and 31 creates a bond to a structural bridging water, which is in turn hydrogen bonded to I154 and A146. The nicotinamide of the SIRT3/AceCS2/carba-NAD⁺ complex makes similar hydrogen bonding contacts to I154, A146 and I230, and with the neighboring structural water. The hydrogen bonding recognition motif of the nicotinamide carboxamide of NAD is mimicked very well by the small molecule sirtuin inhibitors (Compounds 11c, 28 and 31) and explains the observed SAR in Table 5. As a result, a substantial reduction of sirtuin inhibitory activity is observed for compounds that lack the ability to make these critical hydrogen bonds in the nicotinamide C-pocket (Compound 40, where R═CO₂H; Compound 41, where R═CONHCH₃ and Compound 42, where R═H).

With regards to the substrate channel, the thieno[3,2-d]pyrimidine aromatic core lines the top portion of the receptor pocket, along the hydrophobic zinc binding lobe. The thieno[3,2-d]pyrimidine π-stacks with the phenyl ring of F157 and the pyrimidine nitrogen (N1) hydrogen bonds with F157 amide NH donor. The other pyrimidine nitrogen (N3) is sufficiently solvent exposed to facilitate hydrogen bonding with bulk water. The ethyl piperidine of Compound 11c adopts an extended conformation which sits along the top of the hydrophobic cleft of the small structural domain (defined by Y165, F180, I230, I291 and F294), while the arylamide is directed toward the N-acetyllysine substrate channel. The hydrophobic nature of this shelf, explains why the lipophilic piperidines (Compounds 11a, 11e, 20, 28 and 31) are more potent sirtuin inhibitors than the polar piperazine analogs (Compounds 11b, 11d, 24, 30 and 32), where the piperazine nitrogen would be located in the middle of the hydrophobic surface.

Further down the substrate channel, the aryl amide NH hydrogen bonds with V292. In other substrate bound structures (3GLR and 4FVT), V292 forms a hydrogen bond with the N-ε-acetyl lysine from the substrate. For the SIRT1/2/3 inhibitors, a modest improvement in inhibition is observed the more acidic the NH donor that interacts with V292 is. For instance, comparing the SIRT1/2/3 inhibitory activity of the sulfonamide (Compound 25), with acetamide (Compound 20) reveals an 8 to 28 fold improvement in potency. However, the SIRT1/2/3 inhibitors lack of an available NH donor to interact with V292, as exemplified by the pyrrolidine (Compound 34), resulted in only modest changes in sirtuin inhibitory activity.

The X-ray structure also provides an explanation for the SAR of the linker length (n) (see FIG. 3). The ethyl linker (where n=2) for the piperidine (Compounds 20 and 28) optimally aligns the amide NH to hydrogen bond with V292, whereas the methylene (Compounds 23 & 27, where n=1) are presumably too short to optimally make this interaction, resulting in a reduction of potency. The longer propylpiperidine (Compound 29, where n=3) is envisioned to twist to maintain this interaction, but with a slight loss of activity (1.5-3 fold). Lastly, the distal ethylamide substituted on the 2-thiophene on Compound 11c forms a hydrogen bond with Glu296. This ethylamide, extending out of the substrate cavity is solvent exposed, where it is the attachment point to the DNA linker found in the original on-DNA molecules. Removing this portion had little effect on sirtuin inhibitory activity (compare Compounds 19 and 11c) given the sum of the other interactions the small molecule inhibitors make with the catalytic site.

The larger portion of the catalytic site, the space usually occupied by the ribofuranose to the adenine site of NAD⁺, is still largely unoccupied in the SIRT3/Compound 11c, SIRT3/Compound 28 and SIRT3/Compound 31 structures, except for bulk water or crystallization medium. This space may be more efficiently exploited in future designs. The residues that form the NAD⁺ binding pocket are highly conserved between SIRT1/2/3, which likely explains why these compounds are pan inhibitors. Interestingly, several small molecule sirtuin inhibitors that have been described in the literature possess carboxamides. Nicotinamide, EX-527 (Compound 4) and benzamides (e.g. Compound 7) all have a carboxamide which is sensitive to substitution. It would be interesting to determine how these other carboxamide containing sirtuin inhibitors bind and impart their selectivity profiles should they bind similarly in the nicotinamide C-pocket.

Taken together, the observed SAR for thieno[3,2-d]pyrimidine-6-carboxamide based inhibitors of SIRT1/2/3 is in strong agreement with the observed ligand interactions found in the SIRT3/Compound 11c, SIRT3/Compound 28 and SIRT3/Compound 31 structures. Compounds 11c and 28 were generally selective (XC₅₀>10 μM) when broadly profiled against a panel of kinases, nuclear receptors, ion channels, transporters and GPCRs. In addition, they are poor hERG binders (Compounds 11c and 28, >50 μM) and inactive against a variety of CYPs (Compound 28, 1A2, 2C19, 2D6 and 3A4>50 μM, 2C9=7.2 μM). Further, Compound 28 has a low LogD (2.73), high solubility (297 μM) and stability in microsomes (Human CLᵢₙₜ=15.8 μL/min/mg, Mouse CLᵢₙₜ=12.7 μL/min/mg).

The inhibitor Compound 11c and the truncated analogs (Compounds 28 and 31), represent a significant advance over currently available sirtuin inhibitors. Their competitive mode of action has been corroborated by X-ray crystallographic data, and the SAR is in agreement with that structural information. The potency of this novel class of inhibitors make them valuable tools for understanding the biological effects of modulating the deacetylase activity of SIRT1, SIRT2 and SIRT3.

### Example 6

### Acetyl-P65 Assay

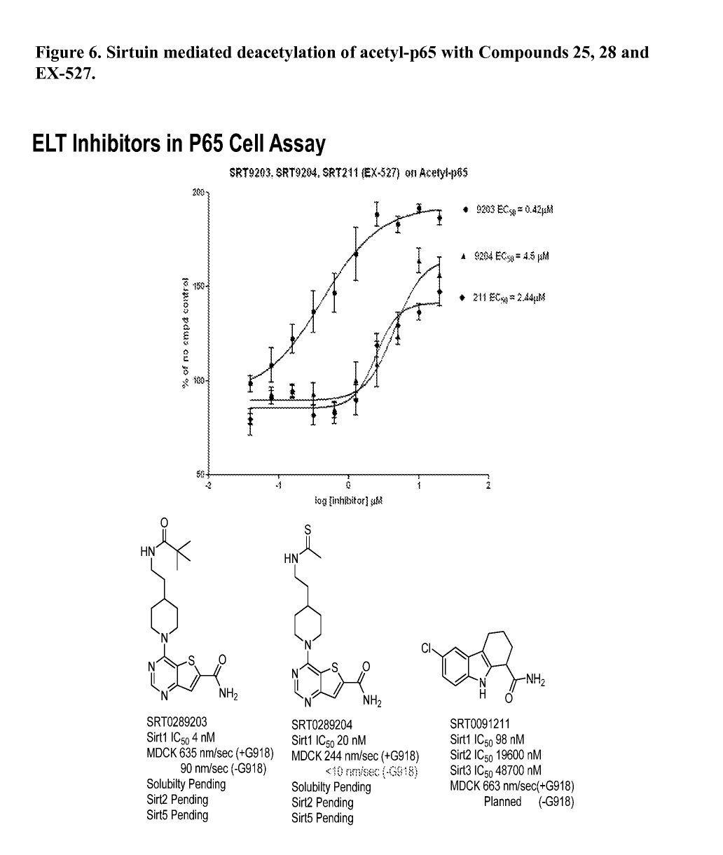

In U2OS cells, Compounds 25 and 28 and Compound 4 (Napper, A. D. et al. (2005) J Med Chem 48, 8045-8054) have been shown to inhibit the sirtuin mediated deacetylation of acetyl-p65 (see FIG. 6).

In certain embodiments, U2OS cells were counted by hemocytometer and diluted to a concentration of 1.5×10⁵ cell/ml. BacMam p65 and BacMam p300-HAT viruses were added to the diluted cells at 1% and 1% (vol/vol). 40 μl aliquots of the cell suspensions containing the viruses were plated onto a 384 well plate with a multi-drop dispenser. After 7 hours, a 2-fold serial dilution of Compounds 25, 28 and Compound 4, hereafter referred to as the test compounds, was carried out in DMSO. The test compounds were subsequently diluted with medium (20-fold) in an intermediate compound plate, and 4 μL of each test compound was transferred by a liquid handler from the intermediate plate to a cell plate. 24 hours post viral transduction, the medium in the cell plate was removed by inverting and flicking the plate, and blotting the plate with paper towels. 30 μl of lysis buffer (25 mM HEPES pH 7.4, 0.5% Triton X-100, 1 mg/ml Dextran 500, 0.1% BSA, 300 mM NaCl, 2 mM MgCl₂, 1×protease inhibitor cocktail) was added to each well to lyse the cells. After incubation of the cells with lysis buffer for 30 minutes at room temp, 10 μl and 3 μl aliquots of the cell lysates were transferred to assay plates, and the assay plate containing 3 μl aliquot of cell lysates was diluted with an additional 7 μl of lysis buffer to obtain a final volume of 10 μl. Acetyl-p65 and total p65 protein in cell lysates were measured using the AlphaScreen assay format (PerkinElmer). The antibodies used to detect acetyl-p65 protein were biotinylated anti-HA antibody (Roche, 12158167001) and anti-acetylated K310-p65 antibody (Abcam, ab19870). The antibodies used to detect total p65 protein were biotinylated anti-HA antibody (Roche, 12158167001) and anti-p65 antibody (Santa Cruz, sc109). 6 μl of the mixtures of diluted antibodies (final concentration at 2 nM each) and protein A coated acceptor beads (final concentration at 20 μg/ml) diluted in detection buffer (25 mM HEPES pH 7.4, 0.5% Triton X-100, 1 mg/ml Dextran 500, 0.1% BSA) were added into the assay plates containing the cell lysates. After incubating the plates at room temp for 2 hrs, 2 μl of streptavidin coated donor beads (diluted in detection buffer to a final concentration 20 μg/ml) were added to the same plates. After incubating the plates for another 2 hrs in the dark, the plates were read by a PHERAstar microplate reader. The data shown in the graph represent the percentage of signals in the test samples relative to the DMSO control samples. In certain embodiments, U205, HEK 293 MSRII cells could be used in the acetyl p65 assays described herein to detect SIRT1/2/3 inhibitors of the present invention.

### Example 7

### Preparation of 4-(4-((dimethylamino)methyl)piperidin-1-yl)thieno[3,2-d]pyrimidine-6-carboxamide (Compound 43)

### Step 1. Synthesis of 4-chlorothieno[3,2-d]pyrimidine-6-carboxylic acid (Compound 13)

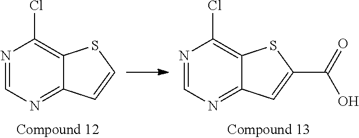 [SMILES: Clc1ncnc2ccsc12 ; O=C(O)c1cc2ncnc(Cl)c2s1]

To a stirring solution of 2,2,6,6-tetramethylpiperidine (1.484 mL, 8.79 mmol) in anhydrous THF (15 mL) at 0° C. under nitrogen was added 2.5 M BuLi in hexanes (3.52 mL, 8.79 mmol) dropwise. The reaction mixture was stirred at 0° C. for 30 minutes, then the mixture was added to a solution of 4-chlorothieno[3,2-d]pyrimidine (Compound 12; 1.00 g, 5.86 mmol) in anhydrous THF (15 mL) at −78° C. dropwise over a period of 30 minutes. The reaction was stirred at −78° C. for 1 hour, and then dry ice (2.58 g, 58.6 mmol) was added to the reaction. The reaction was allowed to warm up to room temperature over a period of 2 hours. The reaction was diluted with EtOAc (100 mL) and washed with 0.1 M HCl. The organic layer was dried over MgSO₄ and evaporated to dryness to obtain 4-chlorothieno[3,2-d]pyrimidine-6-carboxylic acid (Compound 13; 1.1 g, 83%). MS (ESI) calcd for C₇H₃ClN₂O₂S: 213.96. found: 215.0 [M+H].

### Step 2. Synthesis of 4-chlorothieno[3,2-d]pyrimidine-6-carboxamide (Compound 14)

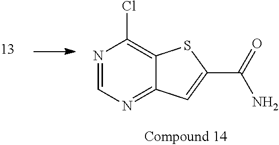 [SMILES: NC(=O)c1cc2ncnc(Cl)c2s1]

To a solution of oxalyl chloride (4.17 mL, 47 mmol) in anhydrous dichloromethane (50 mL) at 0° C. under nitrogen was added DMF (0.8 mL). The solution was stirred at 0° C. for 30 minutes, and then a suspension of 4-chlorothieno[3,2-d]pyrimidine-6-carboxylic acid (13; 5.04 g, 23.5 mmol) in dichloromethane (50 mL) was added dropwise over 10 minutes at 0° C. The reaction mixture was heated to 60° C. for 3.5 hours and concentrated to dryness. The crude acid chloride was dissolved in dioxane (80 mL) and 182 mL (91 mmol) of a solution of 0.5 M ammonia in dioxane was added dropwise over 10 min at 0° C. The reaction mixture was stirred at 0° C. for 30 min, warmed to room temperature, diluted with water (150 mL) and extracted with CH₂Cl₂ (3×). The combined organic layers were washed with water (2×), dilute aq. NaHCO₃, brine and concentrated to dryness. The product was recrystallized from CH₃CN to obtain 4-chlorothieno[3,2-d]pyrimidine-6-carboxamide (Compound 14; 0.842 g). The mother liquor was concentrated to obtain a second crop (Compound 14; 1.499 g) and a third crop (Compound 14; 0.259 g) of 4-chlorothieno[3,2-d]pyrimidine-6-carboxamide for a total of 2.6 g (52%) of Compound 14. MS (ESI) calcd for C₇H₄ClN₃OS: 212.98. found: 214.0 [M+H].

### Step 3. Synthesis of 4-(4-((dimethylamino)methyl)piperidin-1-yl)thieno[3,2-d]pyrimidine-6-carboxamide (Compound 43)

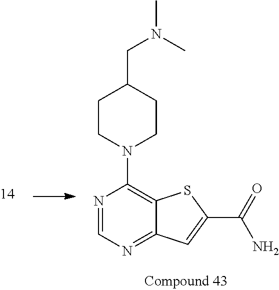 [SMILES: CN(C)CC1CCN(c2ncnc3cc(C(N)=O)sc23)CC1]

A solution of 4-chlorothieno[3,2-d]pyrimidine-6-carboxamide (14; 0.043 g, 0.200 mmol), N,N-dimethyl-1-(piperidin-4-yl)methanamine (0.028 g, 0.200 mmol) and DIEA (42 μL, 0.24 mmol) in NMP (1 mL) was heated to 100° C. for 2 hours. The reaction mixture was concentrated to dryness, and the residue was dissolved in DMSO and purified by mass directed prep-HPLC. The fractions were lyophilized to obtain 4-(4-((dimethylamino)methyl)piperidin-1-yl)thieno[3,2-d]pyrimidine-6-carboxamide as the TFA salt (Compound 43; 0.039 g, 46%). MS (ESI) calcd for C₁₅H₂₁N₅OS: 319.15. found: 320 [M+H].

Compounds 34 and 44-52 of Table 7 were prepared in an analogous manner.

### Example 8

### Preparation of 4-(4-(acetamidomethyl)piperidin-1-yl)thieno[3,2-d]pyrimidine-6-carboxamide (Compound 23)

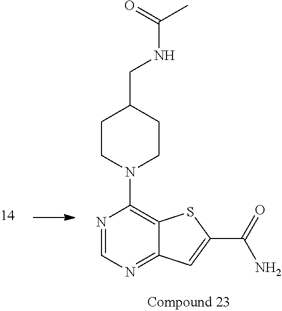 [SMILES: CC(=O)NCC1CCN(c2ncnc3cc(C(N)=O)sc23)CC1]

A solution of 4-chlorothieno[3,2-d]pyrimidine-6-carboxamide (0.043 g, 0.200 mmol) and N-(piperidin-4-ylmethyl)acetamide (14; 0.031 g, 0.200 mmol) in pyridine (1 mL) was heated at 80° C. for 1.5 hours. The reaction mixture was concentrated to dryness, and purified by mass directed prep-HPLC to obtain 4-(4-(acetamidomethyl)piperidin-1-yl)thieno[3,2-d]pyrimidine-6-carboxamide as the TFA salt (Compound 23; 0.052 g, 37%). MS (ESI) calcd for C₁₅H₁₉N₅O₂S: 333.13. found: 334 [M+H].

Compounds 31, 33 and 53 of Table 7 were prepared in an analogous manner.

### Example 9

### Preparation of 4-(4-((methylamino)methyl)piperidin-1-yl)thieno[3,2-d]pyrimidine-6-carboxamide (Compound 55)

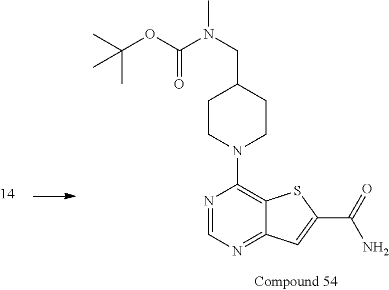 [SMILES: CN(CC1CCN(c2ncnc3cc(C(N)=O)sc23)CC1)C(=O)OC(C)(C)C]

### Step 1. Synthesis of tert-butyl((1-(6-carbamoylthieno[3,2-d]pyrimidin-4-yl)piperidin-4-yl)methyl)(methyl)carbamate (Compound 54)

A solution of 4-chlorothieno[3,2-d]pyrimidine-6-carboxamide (14; 0.043 g, 0.200 mmol) and tert-butyl methylcarbamate (0.200 mmol) in NMP (1 mL) was heated at 100° C. for 2 hours. The reaction mixture was concentrated to dryness, and purified by mass triggered prep HPLC. The fractions were lyophilized to obtain tert-butyl ((1-(6-carbamoylthieno[3,2-d]pyrimidin-4-yl)piperidin-4-yl)methyl)(methyl)carbamate (Compound 54; 0.200 mmol, assumed quantitative) which was used directly in the next step. MS (ESI) calcd for C₁₉H₂₇N₅O₃S: 405.18.

### Step 2. Synthesis of 4-(4-((methylamino)methyl)piperidin-1-yl)thieno[3,2-d]pyrimidine-6-carboxamide (Compound 55)

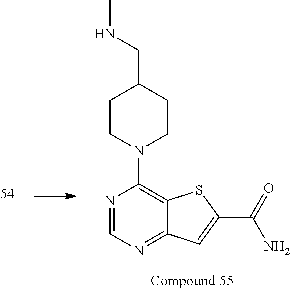 [SMILES: CNCC1CCN(c2ncnc3cc(C(N)=O)sc23)CC1]

Compound 54 (assumed 0.200 mmol) was dissolved CH₂Cl₂ (5 mL) and TFA (0.3 mL) was added. The mixture was stirred at room temperature for 18 hours, concentrated to dryness, dissolved in CH₃CN/H₂O mixture and lyophilized to obtain 4-(4-((methylamino)methyl)piperidin-1-yl)thieno[3,2-d]pyrimidine-6-carboxamide as the TFA salt (Compound 55; 0.082 g, 98%). MS (ESI) calcd for C₁₄H₁₉N₅OS: 305.13. found: 306 [M+H].

Compound 56 of Table 7 was prepared in an analogous manner.

### Example 10

### Preparation of tert-butyl (2-(1-(6-carbamoylthieno[3,2-d]pyrimidin-4-yl)piperidin-4-yl)ethyl)carbamate (Compound 15a)

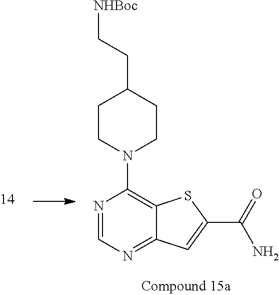 [SMILES: CC(C)(C)OC(=O)NCCC1CCN(c2ncnc3cc(C(N)=O)sc23)CC1]

A solution of 4-chlorothieno[3,2-d]pyrimidine-6-carboxamide (14; 1.30 g, 6.08 mmol), tert-butyl(2-(piperidin-4-yl)ethyl)carbamate (1.39 g, 6.08 mmol) and DIEA (1.05 mL, 6.08 mmol) in CH₃CN (80 mL) was heated at reflux for 1 hour. The reaction mixture was cooled to room temperature and concentrated to dryness. The residue was suspended in MeOH (10 mL) then water (90 mL) was added. The mixture was sonicated and the precipitate was collected by filtration, washed with water and dried under high vacuum to obtain tert-butyl (2-(1-(6-carbamoylthieno[3,2-d]pyrimidin-4-yl)piperidin-4-yl)ethyl)carbamate (Compound 15a; 2.23 g, 90%). MS (ESI) calcd for C₁₉H₂₇N₅O₃S: 405.18. found: 406 [M+H].

Compounds 182, 183 and 184 of Table 7 were prepared in an analagous manner.

### Example 11

### Preparation of 4-(4-(2-aminoethyl)piperidin-1-yl)thieno[3,2-d]pyrimidine-6-carboxamide.HCL salt (Compound 16a)

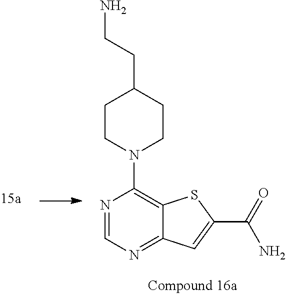 [SMILES: NCCC1CCN(c2ncnc3cc(C(N)=O)sc23)CC1]

To a solution of tert-butyl (2-(1-(6-carbamoylthieno[3,2-d]pyrimidin-4-yl)piperidin-4-yl)ethyl)carbamate (15a; 0.840 g, 2.07 mmol) in CH₂Cl₂ (40 mL) was added trifluoroacetic acid (10 mL). The reaction mixture was stirred for 72 hours, concentrated to dryness and chased with CH₂Cl₂ (2×). The residue was diluted with MeOH, and then a solution of 1.25 M HCl in MeOH (2 mL) was added. To the resulting oil was added diethyl ether (15 mL) and pentane (5 mL). The solution was sonicated to produce solid which was isolated by decantation, and dried under vacuum to obtain 4-(4-(2-aminoethyl)piperidin-1-yl)thieno[3,2-d]pyrimidine-6-carboxamide as the hydrochloride salt (Compound 16a; 1.09 g). MS (ESI) calcd for C₁₄H₁₉N₅OS: 305.13. found: 306 [M+H].

Compound 185 of Table 7 was prepared in an analogous manner.

### Example 12

### Preparation of 4-(4-(2-aminoethyl)piperidin-1-yl)thieno[3,2-d]pyrimidine-6-carboxamide.TFA salt (Compound 16a)

tert-butyl(2-(1-(6-carbamoylthieno[3,2-d]pyrimidin-4-yl)piperidin-4-yl)ethyl)carbamate (15a; 2.23 g, 5.5 mmol) was stirred with 25% TFA in CH2Cl2 (40 mL) for 3 hours. The mixture was concentrated to dryness and triturated with diethyl ether to obtain 4-(4-(2-aminoethyl)piperidin-1-yl)thieno[3,2-d]pyrimidine-6-carboxamide as the TFA salt (16a; 3.74 g, assumed quantitative). MS (ESI) calcd for C₁₄H₁₉N₅OS: 305.13. found: 306 [M+H].

### Example 13

### Preparation of tert-butyl (2-(4-(6-carbamoylthieno[3,2-d]pyrimidin-4-yl)piperazin-1-yl)ethyl)carbamate (Compound 15b)

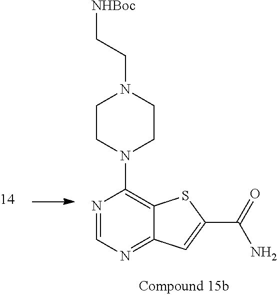 [SMILES: CC(C)(C)OC(=O)NCCN1CCN(c2ncnc3cc(C(N)=O)sc23)CC1]

A solution of 4-chlorothieno[3,2-d]pyrimidine-6-carboxamide (14; 0.250 g, 1.17 mmol), DIEA (245 μL, 1.40 mmol) and tert-butyl (2-(piperazin-1-yl)ethyl)carbamate (0.332 g, 1.40 mmol) in CH₃CN (15 mL) was heated at 60° C. for 18 hours. The reaction mixture was cooled to room temperature and filtered to collect the product as a solid. The solid was washed with CH₃CN (2×10 mL) and dried to obtain tert-butyl (2-(4-(6-carbamoylthieno[3,2-d]pyrimidin-4-yl)piperazin-1-yl)ethyl)carbamate as a white solid (Compound 15b; 0.440 g, 93%). MS (ESI) calcd for C₁₈H₂₆N₆O₃S: 406.18. found: 407 [M+H].

### Example 14

### Preparation of 4-(4-(2-aminoethyl)piperazin-1-yl)thieno[3,2-d]pyrimidine-6-carboxamide (Compound 16b)

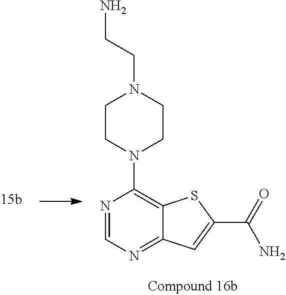 [SMILES: NCCN1CCN(c2ncnc3cc(C(N)=O)sc23)CC1]

A solution of tert-butyl (2-(4-(6-carbamoylthieno[3,2-d]pyrimidin-4-yl)piperazin-1-yl)ethyl)carbamate (15b; 0.440 g, 2.46 mmol) was stirred with 25% TFA in CH₂Cl₂ (8 mL) for 6 hours. The solution was concentrated to dryness and triturated with a mixture of diethyl ether and pentane mixture to obtain 4-(4-(2-aminoethyl)piperazin-1-yl)thieno[3,2-d]pyrimidine-6-carboxamide as the bis-TFA salt (Compound 16b; 0.844 g, 64%), a tan solid. MS (ESI) calcd for C₁₃H₁₈N₆OS: 306.13. found: 307 [M+H].

### Example 15

### Preparation of tert-butyl ((1-(6-carbamoylthieno[3,2-d]pyrimidin-4-yl)piperidin-4-yl)methyl)carbamate (Compound 57)

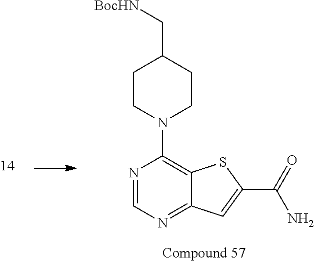 [SMILES: CC(C)(C)OC(=O)NCC1CCN(c2ncnc3cc(C(N)=O)sc23)CC1]

A solution of 4-chlorothieno[3,2-d]pyrimidine-6-carboxamide (14; 0.128 g, 0.6 mmol) and tert-butyl (piperidin-4-ylmethyl)carbamate (0.193 g, 0.9 mmol) in CH₃CN was heated at 80° C. for 18 h. The reaction mixture was concentrated to dryness, suspended in EtOAc, washed with sat. NaHCO₃, water, brine, dried (Na₂SO₄), and concentrated to dryness. The crude product was purified by flash chromatography (0 to 10% MeOH in CH₂Cl₂ gradient) to obtain tert-butyl ((1-(6-carbamoylthieno[3,2-d]pyrimidin-4-yl)piperidin-4-yl)methyl)carbamate (Compound 57; 0.224 g, 95%). MS (ESI) calcd for C₁₈H₂₅N₅O₃S: 391.17. found: 392 [M+H].

Compound 58 of Table 7 was prepared in an analogous manner.

### Example 16

### Preparation of 4-(4-(aminomethyl)piperidin-1-yl)thieno[3,2-d]pyrimidine-6-carboxamide (Compound 59)

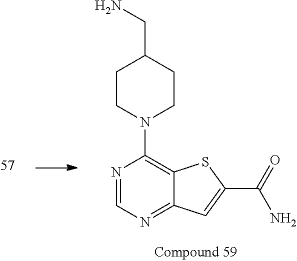 [SMILES: NCC1CCN(c2ncnc3cc(C(N)=O)sc23)CC1]

To a solution of tert-butyl ((1-(6-carbamoylthieno[3,2-d]pyrimidin-4-yl)piperidin-4-yl)methyl)carbamate (57; 0.125 g, 0.32 mmol) in THF (20 mL) and water (5 mL) was added conc. HCl (0.5 mL). The reaction mixture was stirred at room temperature for 3 h and concentrated. The residue was chased with diethyl ether (2×), pentane and dried to obtain 4-(4-(aminomethyl)piperidin-1-yl)thieno[3,2-d]pyrimidine-6-carboxamide (Compound 59; 0.117 g, assumed quantitative). MS (ESI) calcd for C₁₃H₁₇N₅OS: 291.12. found: 292 [M+H].

### Example 17

### Preparation of N¹-(2-(1-(6-carbamoylthieno[3,2-d]pyrimidin-4-yl)piperidin-4-yl)ethyl)-N³-ethylisophthalamide (Compound 11a)

### Step 1. Synthesis of methyl 3-(ethylcarbamoyl)benzoate (Compound 61)

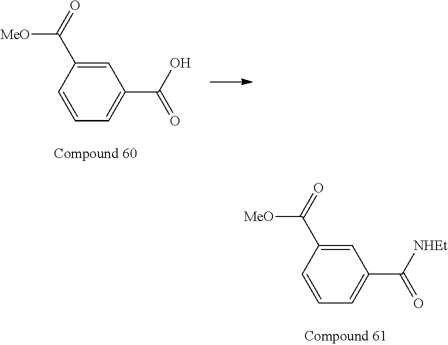

A solution of 3-(methoxycarbonyl)benzoic acid (Compound 60; 1.0 g, 5.55 mmol), HATU (2.53 g, 6.65 mmol), DIEA (1.44 mL, 8.31 mmol) in DMF (15 mL) was stirred at room temperature for 10 min, then ethylamine (70% in water, 8.84 mL) was added and the reaction mixture was stirred at room temperature for 18 hours. The mixture was diluted with ethyl acetate and the organic layer was washed with sat. NaHCO₃, water (3×), dried (Na₂SO₄), concentrated and chased with methanol to obtain methyl 3-(ethylcarbamoyl)benzoate as an orange oil (Compound 61; 1.20 g, assumed quantitative), which was used without further purification in the next step. MS (ESI) calcd for C₁₁H₁₃NO₃: 207.09.

### Step 2. Synthesis of 3-(ethylcarbamoyl)benzoic acid (Compound 62)

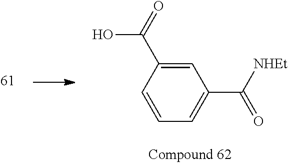

To a solution of methyl 3-(ethylcarbamoyl)benzoate (61; 1.20 g, 5.55 mmol) in methanol (50 mL) was added LiOH (0.666 g, 27.8 mmol) in water (10 mL). The reaction mixture was stirred at room temperature for 72 h, concentrated to dryness, dissolved in water, and acidified with conc. HCl to pH=1-2. The precipitate was collected by filtration, washed with water and dried to obtain 3-(ethylcarbamoyl)benzoic acid (Compound 62; 0.793 g, 74% after 2-steps). MS (ESI) calcd for C₁₀H₁₁NO₃: 193.07. found: 194 [M+H].

### Step 3. Synthesis of N¹-(2-(1-(6-carbamoylthieno[3,2-d]pyrimidin-4-yl)piperidin-4-yl)ethyl)-N³-ethylisophthalamide (Compound 11a)

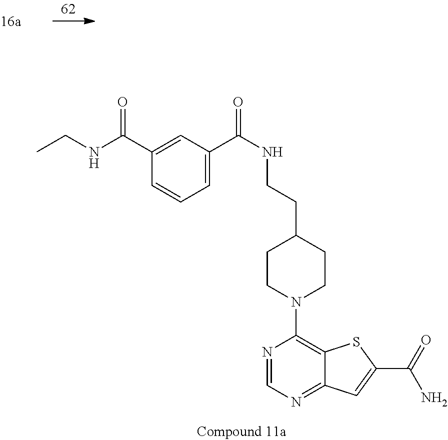 [SMILES: CCNC(=O)c1cccc(C(=O)NCCC2CCN(c3ncnc4cc(C(N)=O)sc34)CC2)c1]

To a mixture of 3-(ethylcarbamoyl)benzoic acid (62; 0.040 g, 0.207 mmol) and HATU (0.076 g, 0.200 mmol) in DMF (3 mL) was added DIEA (0.175 mL, 1.0 mmol). The reaction mixture was stirred for 10 min, and then 4-(4-(2-aminoethyl)piperidin-1-yl)thieno[3,2-d]pyrimidine-6-carboxamide (16a; 0.166 mmol) was added. The reaction mixture was allowed to stir at room temperature overnight, and the product was purified by prep HPLC to obtain N¹-(2-(1-(6-carbamoylthieno[3,2-d]pyrimidin-4-yl)piperidin-4-yl)ethyl)-N³-ethylisophthalamide (Compound 11a; 0.090 g, 77%). MS (ESI) calcd for C₂₄H₂₈N₆O₃S: 480.19. found: 481 [M+H].

Compounds 11b, 11c, 11d and 63 of Table 7 and Compounds 190, 193, 194 of Table 8 were prepared in an analogous manner.

### Example 18

### Preparation of tert-butyl 5-((2-(1-(6-carbamoylthieno[3,2-d]pyrimidin-4-yl)piperidin-4-yl)ethyl)carbamoyl)thiophene-2-carboxylate (Compound 17)

### Step 1. Synthesis of methyl 5-(ethylcarbamoyl)thiophene-2-carboxylate (Compound 65)

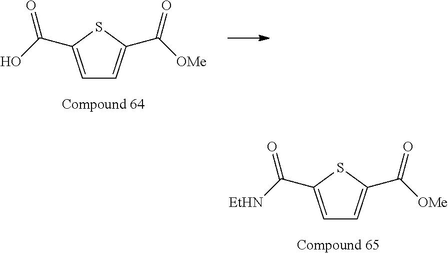

To a solution of 5-(methoxycarbonyl)thiophene-2-carboxylic acid (Compound 64; 0.500 g, 2.68 mmol) and HATU (1.23 g, 3.23 mmol) in DMF (10 mL) was added DIEA (1.16 mL, 6.70 mmol). The reaction mixture was stirred at room temperature for 10 min, and then ethylamine hydrochloride (0.219 g, 2.68 mmol) was added as a solid. The reaction mixture was stirred for 7 h, and another batch of ethylamine hydrochloride (0.219 g, 2.68 mmol) and DIEA (1.16 mL, 6.70 mmol) was added and stirring was continued for a total of 24 h. To the reaction mixture was added sat. NaHCO₃ (30 mL) and water (50 mL). The precipitated product was collect by filtration, and washed with water to obtain methyl 5-(ethylcarbamoyl)thiophene-2-carboxylate as a tan solid (Compound 65; 0.555 g, 97%). MS (ESI) calcd for C₉H₁₁NO₀S: 213.05. found: 214 [M+H].

### Step 2. Synthesis of 5-(ethylcarbamoyl)thiophene-2-carboxylic acid (Compound 66)

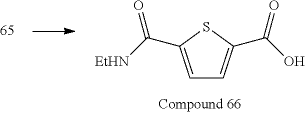

To a solution of methyl 5-(ethylcarbamoyl)thiophene-2-carboxylate (Compound 65; 0.555 g, 2.60 mmol) in THF (5 mL) was added a solution of LiOH (0.124 g, 5.18 mmol) in water (5 mL). The reaction mixture was stirred at room temperature for 72 h, concentrated to dryness, dissolved in water, and acidified with conc. HCl to pH=1. The precipitate was collected by filtration, washed with water and dried under vacuum to obtain 5-(ethylcarbamoyl)thiophene-2-carboxylic acid as an off white solid (Compound 66; 0.221 g, 43%). MS (ESI) calcd for C₈H₉NO₃S: 199.03. found: 200 [M+H].

### Step 3. Synthesis of tert-butyl 5-((2-(1-(6-carbamoylthieno[3,2-d]pyrimidin-4-yl)piperidin-4-yl)ethyl)carbamoyl)thiophene-2-carboxylate (Compound 17)

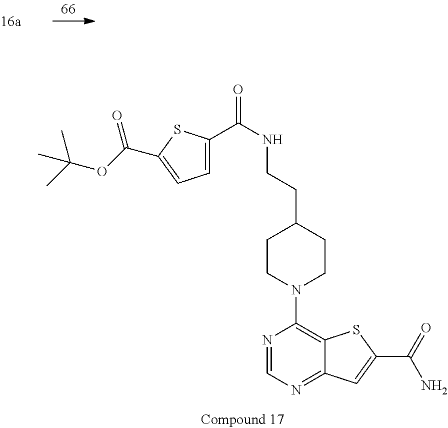 [SMILES: CC(C)(C)OC(=O)c1ccc(C(=O)NCCC2CCN(c3ncnc4cc(C(N)=O)sc34)CC2)s1]

A solution of 5-(tert-butoxycarbonyl)thiophene-2-carboxylic acid (Compound 66; 0.057 g, 0.25 mmol), HATU (0.114 g, 3.0 mmol) and DIEA (248 μL, 2.0 mmol) in DMF (3 mL) was stirred at room temperature for 5 min. 4-(4-(2-Aminoethyl)piperidin-1-yl)thieno[3,2-d]pyrimidine-6-carboxamide 2,2,2-trifluoroacetate (Compound 16a; 0.105 g, 0.25 mmol) was added, and the reaction mixture was stirred at room temperature overnight. The reaction mixture was diluted with water, extracted with CH₂Cl₂ (3×), washed with aq. NaHCO₃ (sat.), brine and concentrated to dryness. The crude material was purified by prep-HPLC, and the fractions were lyophilized to obtain tert-butyl 5-((2-(1-(6-carbamoylthieno[3,2-d]pyrimidin-4-yl)piperidin-4-yl)ethyl)carbamoyl)thiophene-2-carboxylate (Compound 17; 0.048 g, 37%). MS (ESI) calcd for C₂₄H₂₉N₅O₄S₂: 515.17. found: 516 [M+H].

Compound 67 of Table 7 was prepared in an analogous manner.

### Example 19

### Preparation of 5-((2-(1-(6-carbamoylthieno[3,2-d]pyrimidin-4-yl)piperidin-4-yl)ethyl)carbamoyl)thiophene-2-carboxylic acid (Compound 18)

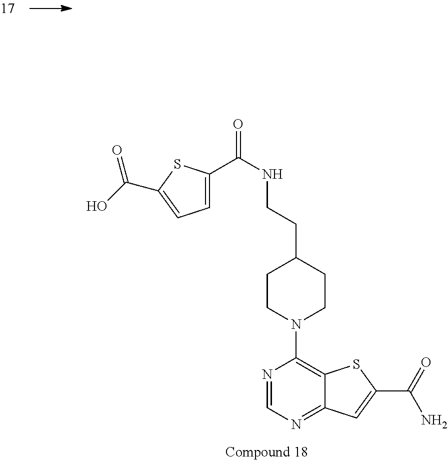 [SMILES: NC(=O)c1cc2ncnc(N3CCC(CCNC(=O)c4ccc(C(=O)O)s4)CC3)c2s1]

A solution of tert-butyl 5-((2-(1-(6-carbamoylthieno[3,2-d]pyrimidin-4-yl)piperidin-4-yl)ethyl)carbamoyl)thiophene-2-carboxylate (Compound 17; 0.0341 g, 0.066 mmol) in 1:1 TFA/CH₂Cl₂ (5 mL) was stirred overnight. The reaction mixture was concentrated to dryness, triturated with a mixture of diethyl ether/pentane and dried under vacuum to obtain the title compound as a solid (Compound 18; 0.0216 g, 71%). MS (ESI) calcd for C₂₀H₂₁N₅O₄S₂: 459.10. found: 460 [M+H].

### Example 20

### Preparation of 4-(4-(2-(thiophene-2-carboxamido)ethyl)piperidin-1-yl)thieno[3,2-d]pyrimidine-6-carboxamide (Compound 19)

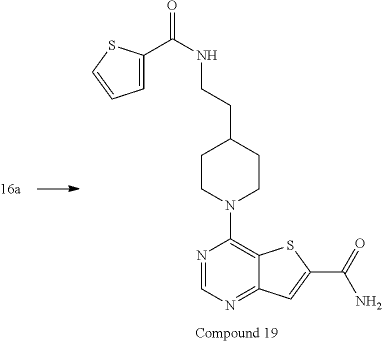 [SMILES: NC(=O)c1cc2ncnc(N3CCC(CCNC(=O)c4cccs4)CC3)c2s1]

To a mixture of 4-[4-(2-aminoethyl)-1-piperidinyl]thieno[3,2-d]pyrimidine-6-carboxamide (Compound 16a; 0.025 g, 0.060 mmol) and HATU (0.0272 g, 0.072 mmol) in N,N-Dimethylformamide (DMF) (3 mL) was added thiophene-2-carboxylic acid (0.0076 g, 0.06 mmol) and DIPEA (0.052 mL, 0.298 mmol). The reaction mixtures were allowed to stir at room temperature overnight. The product was purified by prep HPLC to obtain 4-(4-(2-(thiophene-2-carboxamido)ethyl)piperidin-1-yl)thieno[3,2-d]pyrimidine-6-carboxamide (Compound 19; 0.025 g, 81%). MS (ESI) calcd for C₁₉H₂₁N₅O₂S₂: 415.11. found: 416 [M+H].

Compound 20 of Table 7 was prepared in an analogous manner.

### Example 21

### Preparation of 4-(4-(2-ethanethioamidoethyl)piperidin-1-yl)thieno[3,2-d]pyrimidine-6-carboxamide (Compound 25)

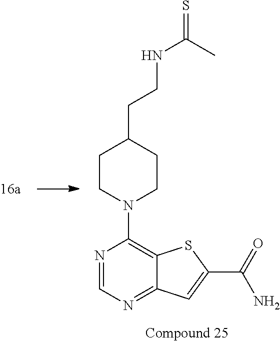 [SMILES: CC(=S)NCCC1CCN(c2ncnc3cc(C(N)=O)sc23)CC1]

To a solution of 4-(4-(2-aminoethyl)piperidin-1-yl)thieno[3,2-d]pyrimidine-6-carboxamide 2,2,2-trifluoroacetate (16a; 0.428 g, 1.0 mmol), and Na₂CO₃ (0.845 g, 8 mmol) in EtOH (10 mL) and water (50 mL) was added ethyl dithioacetate (136 μL, 1.2 mmol). The reaction mixture was stirred at room temperature for 6 h, concentrated to dryness. Water (50 mL) was added and the solid was collected by filtration. The solid was triturated with methanol and dried under vacuum to obtain 44442-ethanethioamidoethyl)piperidin-1-yl)thieno[3,2-d]pyrimidine-6-carboxamide (Compound 25; 0.137 g, 38%). MS (ESI) calcd for C₁₆H₂₁N₅OS₂: 363.12. found: 364 [M+H]. Compound 26 of Table 7 was prepared in an analogous manner.

### Example 22

### Preparation of 4-(4-(pivalamidomethyl)piperidin-1-yl)thieno[3,2-d]pyrimidine-6-carboxamide (Compound 27)

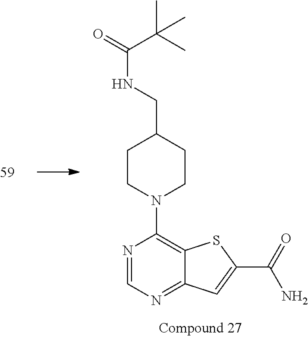 [SMILES: CC(C)(C)C(=O)NCC1CCN(c2ncnc3cc(C(N)=O)sc23)CC1]

4-(4-(aminomethyl)piperidin-1-yl)thieno[3,2-d]pyrimidine-6-carboxamide hydrochloride (59; 0.0915 g, 0.28 mmol) was suspended in EtOAc (20 mL) and water (5 mL). Sodium carbonate (150 mg, 1.39 mmol) was added, followed by pivaoyl chloride (69 μL, 0.56 mmol). The reaction mixture was stirred at room temperature overnight and solid product develops. The reaction mixture was evaporated to remove the organic layer, and solids were collected by filtration, washed with water and dried to obtain 4-(4-(pivalamidomethyl)piperidin-1-yl)thieno[3,2-d]pyrimidine-6-carboxamide (Compound 27; 0.085 g, 81%). MS (ESI) calcd for C₁₈H₂₅N₅O₂S: 375.17. found: 376 [M+H]. Compound 186 of Table 7 was prepared in an analagous manner.

### Example 23

### Preparation of 4-(4-(2-pivalamidoethyl)piperidin-1-yl)thieno[3,2-d]pyrimidine-6-carboxamide (Compound 28)

### Step 1. Synthesis of tert-butyl 4-(2-pivalamidoethyl)piperidine-1-carboxylate (Compound 69)

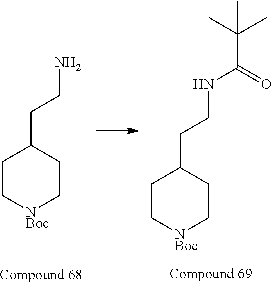 [SMILES: CC(C)(C)OC(=O)N1CCC(CCN)CC1 ; CC(C)(C)OC(=O)N1CCC(CCNC(=O)C(C)(C)C)CC1]

To a solution of tert-butyl 4-(2-aminoethyl)piperidine-1-carboxylate (Compound 68; 1.0 g, 4.38 mmol) and sodium carbonate (1.39 g, 13.1 mmol) in ethyl acetate (15 mL) and water (5 mL) was added pivaloyl chloride (1.07 mL, 8.70 mmol), and the reaction mixture was stirred at room temperature for 18 h. The reaction mixture was diluted with ethyl acetate, and the organic layer was washed with water, brine, dried (Na₂SO₄) and concentrated to obtain tert-butyl 4-(2-pivalamidoethyl)piperidine-1-carboxylate (Compound 69; assumed quantitative).

### Step 2. Synthesis of N-(2-(piperidin-4-yl)ethyl)pivalamide (Compound 70)

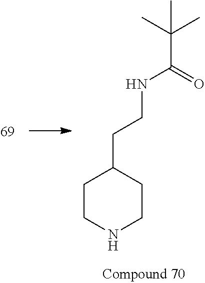

tert-butyl 4-(2-pivalamidoethyl)piperidine-1-carboxylate (69; assumed 4.38 mmol) was diluted with THF (80 mL) and stirred with conc. HCl (3 mL) over night. The reaction mixture was concentrated to dryness, diluted with ethyl acetate and water. The mixture was made basic (pH=14), and the water layer was extracted with ethyl acetate (1×) and CH₂Cl (3×). The organic layers were dried (MgSO₄) and concentrated to afford N-(2-(piperidin-4-yl)ethyl)pivalamide (Compound 70; 0.831 g, 89% after 2-steps). MS (ESI) calcd for C₁₂H₂₄N₂O: 212.19. found: 213 [M+H].

### Step 3. Synthesis of 4-(4-(2-pivalamidoethyl)piperidin-1-yl)thieno[3,2-d]pyrimidine-6-carboxamide (Compound 28)

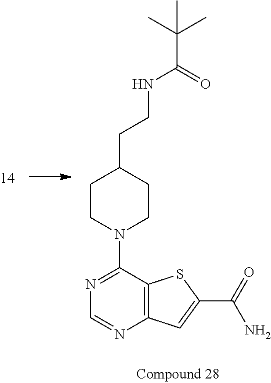 [SMILES: CC(C)(C)C(=O)NCCC1CCN(c2ncnc3cc(C(N)=O)sc23)CC1]

A solution of 4-chlorothieno[3,2-d]pyrimidine-6-carboxamide (14; 0.534 g, 2.5 mmol) and N-(2-(piperidin-4-yl)ethyl)pivalamide (0.530 g, 2.5 mmol) and DIEA (866 μL, 5 mmol) in CH₃CN (30 mL) was heated at 80° C. overnight. The reaction mixture was concentrated and purified on silica gel chromatography (0 to 10% MeOH gradient in CH₂Cl₂) to obtain 4-(4-(2-pivalamidoethyl)piperidin-1-yl)thieno[3,2-d]pyrimidine-6-carboxamide (Compound 28; 0.727 g, 75%) as a yellow solid MS (ESI) calcd for C₁₉H₂₇N₅O₂S: 389.19. found: 390 [M+H].

### Example 24

### Preparation of 4-(4-(3-pivalamidopropyl)piperidin-1-yl)thieno[3,2-d]pyrimidine-6-carboxamide (Compound 29)

### Step 1. Synthesis of 2-(3-(pyridin-4-yl)propyl)isoindoline-1,3-dione (Compound 72)

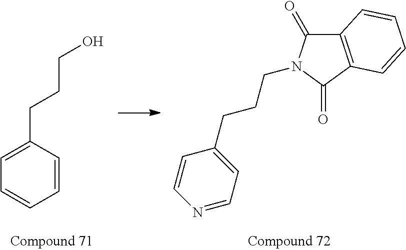 [SMILES: OCCCc1ccccc1 ; O=C1c2ccccc2C(=O)N1CCCc1ccncc1]

To a stirred solution of 3-(pyridin-4-yl)propan-1-ol (Compound 71; 5.0 g, 36.5 mmol), phthalimide (5.25 g, 36.5 mmol) and triphenylphosphine (12.25 g, 38.0 mmol) in THF (100 mL) at 0° C. was added DIAD (8.1 g, 43.8 mmol) dropwise. The reaction mixture was allowed to slowly warm to room temperature and then stirred overnight. The mixture was diluted with 0.1N HCl and washed with diethyl ether. The aqueous extract was made basic with 6N sodium hydroxide and extracted with EtOAc. The organic extract was washed with 1N sodium hydroxide and water, dried (MgSO₄), and concentrated to obtain the crude 2-(3-(pyridin-4-yl)propyl)isoindoline-1,3-dione (Compound 72; 10 g, assumed quantitative) which was used directly in the next step. MS (ESI) calcd for C₁₆H₁₄N₂O₂: 266.11.

### Step 2. Synthesis of 3-(pyridin-4-yl)propan-1-amine (Compound 73)

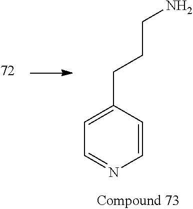

To a solution of 2-(3-(pyridin-4-yl)propyl)isoindoline-1,3-dione (72; 10 g crude, assumed 36.5 mmol) in MeOH (50 mL) was added hydrazine hydrate (5.5 g, 110 mmol). The mixture was stirred at room temperature for 18 h, filtered and the filtrate was concentrated to an oil. The oil was triturated with chloroform, filtered, and the filtrate was concentrated to obtain 3-(pyridin-4-yl)propan-1-amine (Compound 73; 4.0 g, 80%) which was used directly in the next step. MS (ESI) calcd for C₈H₁₂N₂: 136.10.

### Step 3. Synthesis of tert-butyl (3-(pyridin-4-yl)propyl)carbamate (Compound 74)

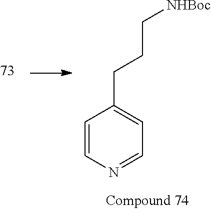 [SMILES: CC(C)(C)OC(=O)NCCCc1ccncc1]

To a solution of 3-(pyridin-4-yl)propan-1-amine (73; 4 g, 29.4 mmol) and triethylamine (6.06 g, 60 mmol) in THF (200 mL) was added dropwise di-tert-butyl dicarbonate (7.8 g, 36.0 mmol). The reaction mixture was stirred at room temperature overnight. Water and ethyl acetate were added, and the aqueous layer was extracted with ethyl acetate (3×). The combined organic layers were dried, concentrated and purified by column chromatography (1% MeOH in CH₂Cl₂) to obtain tert-butyl (3-(pyridin-4-yl)propyl)carbamate (Compound 74; 6.0 g, 88%) which was used without further purification. MS (ESI) calcd for C₁₃H₂₀N₂O₂: 236.15.

### Step 4. Synthesis of tert-butyl (3-(piperidin-4-yl)propyl)carbamate (Compound 75)

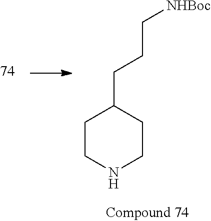 [SMILES: CC(C)(C)OC(=O)NCCCC1CCNCC1]

A solution of tert-butyl (3-(pyridin-4-yl)propyl)carbamate (74; 6.0 g, 25.3 mmol) in of 90% acetic acid (80 mL) was treated with PtO₂ (0.600 g). The mixture was stirred under an atmosphere of hydrogen (50 psi) at 40° C. for 12 h. The catalyst was removed by filtration, and the solvent was evaporated. The residue was dissolved in water, and was adjusted with 1N NaOH to pH 11. The aqueous layer was extracted with CH₂Cl₂ (3×) and the combined organic layers were dried (Na₂SO₄), and concentrated to obtain tert-butyl (3-(piperidin-4-.yl)propyl)carbamate (Compound 75; 3.0 g, 50%). MS (ESI) calcd for C₁₃H₂₆N₂O₂: 242.20. found: 243 [M+H].

### Step 5. Synthesis of tert-butyl (3-(1-(6-carbamoylthieno[3,2-d]pyrimidin-4-yl)piperidin-4-yl)propyl)carbamate (Compound 76)

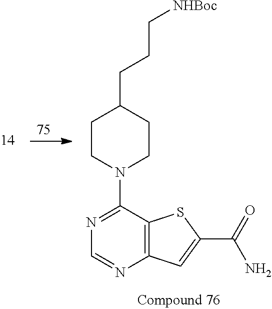 [SMILES: CC(C)(C)OC(=O)NCCCC1CCN(c2ncnc3cc(C(N)=O)sc23)CC1]

A solution of 4-chlorothieno[3,2-d]pyrimidine-6-carboxamide (14; 0.176 g, 0.82 mmol), tert-butyl (3-(pyridin-4-yl)propyl)carbamate (75; 0.200 g, 0.82 mmol) and DIEA (573 μL, 3.3 mmol) in CH₃CN (20 mL) was heated at 80° C. for 20 h. The reaction mixture was concentrated to dryness, and purified by column chromatography (0 to 10% MeOH gradient in CH₂Cl₂) to obtain tert-butyl (3-(1-(6-carbamoylthieno[3,2-d]pyrimidin-4-yl)piperidin-4-yl)propyl)carbamate (Compound 76; 0.216 g, 62%). MS (ESI) calcd for C₂₀H₂₉N₅O₃S: 419.20. found: 420 [M+H].

### Step 6. Synthesis of 4-(4-(3-aminopropyl)piperidin-1-yl)thieno[3,2-d]pyrimidine-6-carboxamide 2,2,2-trifluoroacetate (Compound 77)

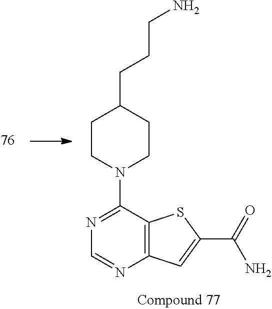 [SMILES: NCCCC1CCN(c2ncnc3cc(C(N)=O)sc23)CC1]

A solution of tert-butyl (3-(1-(6-carbamoylthieno[3,2-d]pyrimidin-4-yl)piperidin-4-yl)propyl)carbamate (76; 0.200 g, 0.476 mmol) in TFA (2.5 mL) in CH₂Cl₂ (7.5 mL) was stirred at room temperature for 4 hours. The reaction mixture was concentrated to dryness, chased with diethyl ether and pentane and dried under vacuum to obtain 4-(4-(3-aminopropyl)piperidin-1-yl)thieno[3,2-d]pyrimidine-6-carboxamide 2,2,2-trifluoroacetate (Compound 77; 0.373 g, assumed quantitative), as an orange solid, which was used without further purification. MS (ESI) calcd for C₁₅H₂₁N₅OS: 319.15. found: 320 [M+H].

### Step 7. Synthesis of 4-(4-(3-pivalamidopropyl)piperidin-1-yl)thieno[3,2-d]pyrimidine-6-carboxamide (Compound 29)

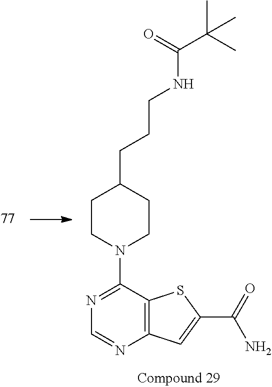 [SMILES: CC(C)(C)C(=O)NCCCC1CCN(c2ncnc3cc(C(N)=O)sc23)CC1]

To a solution of 4-(4-(3-aminopropyl)piperidin-1-yl)thieno[3,2-d]pyrimidine-6-carboxamide 2,2,2-trifluoroacetate (77; 0.100 g, 0.230 mmol) and sodium carbonate (0.121 g, 1.41 mmol) in EtOAc (5 mL) and water (2 mL) was stirred at room temperature for 5 min. Pivaloyl chloride (30 μl, 243 mmol) was added and the reaction mixture was stirred at room temperature for 18 h. The layers were separated, and the organic layer was washed with water, dried (Na₂SO₄) and concentrated to dryness. The crude product was recrystallized in CH₃CN to obtain 4-(4-(3-Pivalamidopropyl)piperidin-1-yl)thieno[3,2-d]pyrimidine-6-carboxamide (Compound 29; 0.0385 g, 41%). MS (ESI) calcd for C₂₀H₂₉N₅O₂S: 403.20. found: 404 [M+H].

### Example 25

### Preparation of 4-(4-(2-pivalamidoethyl)piperazin-1-yl)thieno[3,2-d]pyrimidine-6-carboxamide (Compound 30)

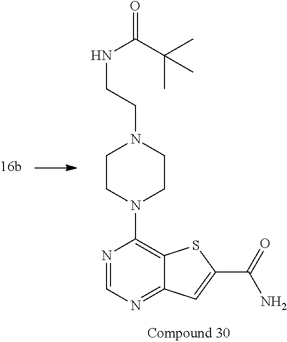 [SMILES: CC(C)(C)C(=O)NCCN1CCN(c2ncnc3cc(C(N)=O)sc23)CC1]

To a solution of 4-(4-(2-aminoethyl)piperazin-1-yl)thieno[3,2-d]pyrimidine-6-carboxamide bis(2,2,2-trifluoroacetate) (16b; 0.107 g, 0.200 mmol) in ethyl acetate (5 mL) and water (2 mL) was added sodium carbonate (212 mg, 2.0 mmol) followed by pivaloyl chloride (36 μL, 0.293 mmol). The reaction mixture was stirred at room temperature overnight, concentrated to dryness, suspended in water and filtered to obtain 4-(4-(2-pivalamidoethyl)piperazin-1-yl)thieno[3,2-d]pyrimidine-6-carboxamide (Compound 30; 0.062 g, 81%) as a white solid. MS (ESI) calcd for C₁₈H₂₆N₆O₂S: 390.18. found: 391 [M+H].

Compound 24 of Table 7 was prepared in an analogous manner by substituting acetyl chloride for pivaloyl chloride.

### Example 26

### Preparation of 4-(4-(5,5-dimethyl-1,3-dioxan-2-yl)piperidin-1-yl)thieno[3,2-d]pyrimidine-6-carboxamide (Compound 78)

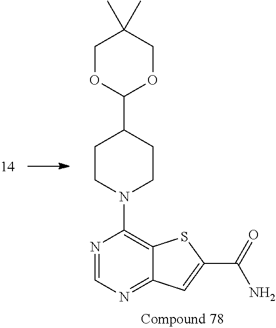 [SMILES: CC1(C)COC(C2CCN(c3ncnc4cc(C(N)=O)sc34)CC2)OC1]

A solution of 4-chlorothieno[3,2-d]pyrimidine-6-carboxamide (14; 0.712 g, 3.33 mmol) and 4-(5,5-dimethyl-1,3-dioxan-2-yl)piperidine oxylate (1.06 g, 3.66 mmol) and DIEA (1.73 mL, 10 mmol) in CH₃CN (30 mL) was heated at 80° C. overnight. The reaction mixture was concentrated to dryness. Aqueous NaHCO₃ (sat) was added, and the solution was extracted with ethyl acetate (2×), and filtered to recover the rag layer (0.516 g of crude product which was reserved). The organic layer was washed with water, brine, dried (Na₂SO₄) and concentrated to obtain 0.715 g of crude product. The crude products were combined and purified by column chromatography (0 to 10% MeOH in CH₂Cl₂ gradient) to obtain 4-(4-(5,5-dimethyl-1,3-dioxan-2-yl)piperidin-1-yl)thieno[3,2-d]pyrimidine-6-carboxamide (Compound 78; 0.926 g, 74%). MS (ESI) calcd for C₁₈H₂₄N₄O₃S: 376.16. found: 377 [M+H].

### Example 27

### Preparation of 4-(4-(2-(methylsulfonamido)ethyl)piperazin-1-yl)thieno[3,2-d]pyrimidine-6-carboxamide 2,2,2-trifluoroacetate (Compound 32)

### Step 1. Synthesis of tert-butyl 4-(2-(methylsulfonamido)ethyl)piperazine-1-carboxylate (Compound 79)

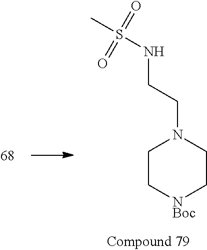 [SMILES: CC(C)(C)OC(=O)N1CCN(CCNS(C)(=O)=O)CC1]

To a solution of tert-butyl 4-(2-aminoethyl)piperazine-1-carboxylate (68; 0.400 g, 1.74 mmol) in CH₂Cl₂ (10 mL) was added pyridine (423 μL, 5.22 mmol), followed by methanesulfonyl chloride (271 μL, 3.53 mmol). The reaction mixture was stirred at room temperature for 18 hours, concentrated to dryness, dissolved in CH₂Cl₂, washed with sat. NaHCO₃, brine, dried (Na₂SO₄) and concentrated to afford tert-butyl 4-(2-(methylsulfonamido)ethyl)piperazine-1-carboxylate (Compound 79; 0.471 g, 88%). MS (ESI) calcd for C₁₂H₂₅N₃O₄S: 307.16. found: 308 [M+H].

### Step 2. Synthesis of N-(2-(piperazin-1-yl)ethyl)methanesulfonamide bis-2,2,2-trifluoroacetate (Compound 80)

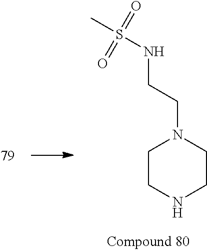

The oily residue, tert-butyl 4-(2-(methylsulfonamido)ethyl)piperazine-1-carboxylate (79; 0.471 g, 1.53 mmol), was dissolved in CH₂Cl₂ (10 mL) and trifluoroacetic acid (2 mL) was added. The reaction mixture was stirred at room temperature overnight, concentrated to dryness, triturated with a diethyl ether, pentane/diethyl ether and then diethyl ether, and dried under vacuum. To obtain N-(2-(piperazin-1-yl)ethyl)methanesulfonamide bis-2,2,2-trifluoroacetate (Compound 80; 0.706 g, assumed quantitative). MS (ESI) calcd for C₇H₁₇N₃O₂S: 207.10. found: 208 [M+H].

### Step 3. Synthesis of 4-(4-(2-(Methylsulfonamido)ethyl)piperazin-1-yl)thieno[3,2-d]pyrimidine-6-carboxamide 2,2,2-trifluoroacetate (Compound 32)

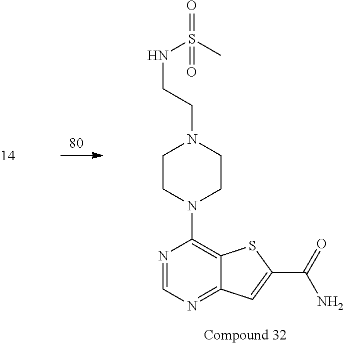 [SMILES: CS(=O)(=O)NCCN1CCN(c2ncnc3cc(C(N)=O)sc23)CC1]

A solution of 4-chlorothieno[3,2-d]pyrimidine-6-carboxamide (14; 0.4 mmol, 0.085 g), N-(2-(piperazin-1-yl)ethyl)methanesulfonamide bis(2,2,2-trifluoroacetate) (80; 0.48 mmol, 0.146 g) and DIEA (208 μL, 1.2 mmol) in CH₃CN and was heated to 60° C. for 18 hrs. The reaction mixture was concentrated to dryness, triturated with methanol and the solid was collected by filtration. The product was purified by Prep-HPLC and lyophilized to afford 4-(4-(2-(methylsulfonamido)ethyl)piperazin-1-yl)thieno[3,2-d]pyrimidine-6-carboxamide (Compound 32; 0.030 g, 15%). MS (ESI) calcd for C₁₄H₂₀N₆O₃S₂: 384.10. found: 385 [M+H].

### Example 28

### Preparation 4-(4-(2-(Pyrrolidin-1-yl)ethyl)piperazin-1-yl)thieno[3,2-d]pyrimidine-6-carboxamide bis(2,2,2-trifluoroacetate) (Compound 35)

### Step 1. Synthesis of 4-(4-(2-hydroxyethyl)piperazin-1-yl)thieno[3,2-d]pyrimidine-6-carboxamide (Compound 44)

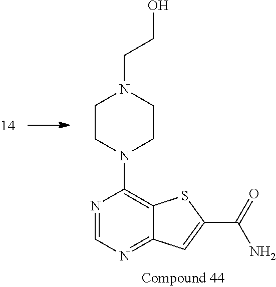 [SMILES: NC(=O)c1cc2ncnc(N3CCN(CCO)CC3)c2s1]

A solution of 4-chlorothieno[3,2-d]pyrimidine-6-carboxamide (14; 0.100 g, 0.468 mmol) and 2-(piperazin-1-yl)ethanol (0.073 g, 0.56 mmol) and DIEA (122 μL, 0.7 mmol) in CH₃CN was heated at 60° C. for 18 h. The reaction mixture was concentrated to dryness, resuspended in dilute aq. NaHCO₃ and collected by filtration. The filter cake was dried under vacuum to obtain 4-(4-(2-hydroxyethyl)piperazin-1-yl)thieno[3,2-d]pyrimidine-6-carboxamide (Compound 44; 0.111 g, 77%). MS (ESI) calcd for C₁₃H₁₇N₅O₂S: 307.37. found: 308 [M+H].

### Step 2. Synthesis of 4-(4-(2-(pyrrolidin-1-yl)ethyl)piperazin-1-yl)thieno[3,2-d]pyrimidine-6-carboxamide (Compound 35)

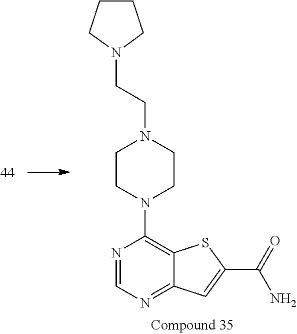 [SMILES: NC(=O)c1cc2ncnc(N3CCN(CCN4CCCC4)CC3)c2s1]

To 4-(4-(2-hydroxyethyl)piperazin-1-yl)thieno[3,2-d]pyrimidine-6-carboxamide (44; 0.111 g, 0.36 mmol) in CH₂Cl₂ was added thionyl chloride (1 mL) and DMF (3 drops). The reaction mixture was stirred at room temperature for 6 h and concentrated to dryness. The residue was resuspended in CH₂Cl₂ and pyrrolidine (1 mL) was added. The mixture was stirred at room temperature for 4 days and concentrated to dryness. The residue was suspended in minimal amount of methanol and water was added. The volume was reduced by 50% and the brown solid was collected by filtration. The mother liquor was concentrated and purified on prep-HPLC and lyophilized to afford 4-(4-(2-(pyrrolidin-1-yl)ethyl)piperazin-1-yl)thieno[3,2-d]pyrimidine-6-carboxamide (Compound 35; 0.024 g, 11%). MS (ESI) calcd for C₁₇H₂₄N₆OS: 402.18. found: 403 [M+H].

### Example 29

### Preparation of 4-(4-(2-pivalamidoethyl)piperidin-1-yl)furo[3,2-d]pyrimidine-6-carboxamide (Compound 36)

### Step 1. Synthesis of 4-methoxyfuro[3,2-d]pyrimidine (Compound 82)

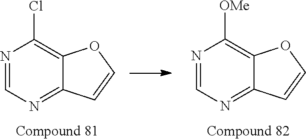 [SMILES: Clc1ncnc2ccoc12 ; COc1ncnc2ccoc12]

To a solution of 4-chlorofuro[3,2-d]pyrimidine (Compound 81; 1.0 g, 6.5 mmol) in MeOH (30 mL) was added sodium methoxide (0.7 g, 13 mmol). The reaction mixture was stirred at 80° C. for 4 hours. The reaction mixture was poured into water, extracted with ethyl acetate and concentrated to obtain 4-methoxyfuro[3,2-d]pyrimidine (Compound 82; 0.600 g, 62%). MS (ESI) calcd for C₇H₆N₂O₂: 150.04.

### Step 2. Synthesis of 4-methoxyfuro[3,2-d]pyrimidine-6-carboxylic acid (Compound 83)

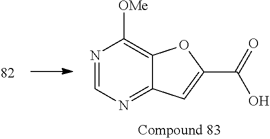 [SMILES: COc1ncnc2cc(C(=O)O)oc12]

To a solution of 4-methoxyfuro[3,2-d]pyrimidine (Compound 82; 0.300 g, 2.0 mmol) in THF, at −40° C., was added 2.5M n-BuLi in hexane (1.2 mL, 3 mmol) dropwise. The reaction was stirred at −40° C. for 30 min and added into dry CO₂ in ether. The reaction mixture was poured into water, with stirring, and the aqueous layer was separated. The aqueous layer was washed with ether. The combined organic layers were extracted with water. The combined aqueous layers were acidified with conc. HCl and extracted with ethyl acetate. The ethyl acetate layers were dried, filtered and concentrated in vacuo to afford 4-methoxyfuro[3,2-d]pyrimidine-6-carboxylic acid as a yellow solid (Compound 83; 0.291 g, 80%). MS (ESI) calcd for C₈H₆N₂O₄: 194.03.

### Step 3. Synthesis of 4-chlorofuro[3,2-d]pyrimidine-6-carboxamide (Compound 84)

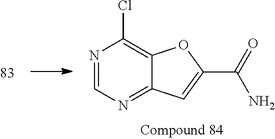 [SMILES: NC(=O)c1cc2ncnc(Cl)c2o1]

To a solution of the above 4-methoxyfuro[3,2-d]pyrimidine-6-carboxylic acid (83; 3 g, 15 mmol), benzyltriethyl ammonium chloride (7 g, 31 mmol) and dimethyl aniline (3 mL, 24 mmol) in acetonitrile (70 mL) at 60° C. was added phosphorous oxychloride (10 mL). The mixture was stirred at 60° C. for 4 h. The reaction mixture was concentrated in vacuo, the residue was dissolved in THF, and ammonium hydroxide solution was added until pH=9. The solid was filtered and dried to obtain 4-chlorofuro[3,2-d]pyrimidine-6-carboxamide (Compound 84; 1.0 g, 33%). MS (ESI) calcd for C₇H₄ClN₃O₂: 197.00. found: 198 [M+H].

### Step 4. Synthesis of tert-butyl (2-(1-(6-carbamoylfuro[3,2-d]pyrimidin-4-yl)piperidin-4-yl)ethyl)carbamate (Compound 85)

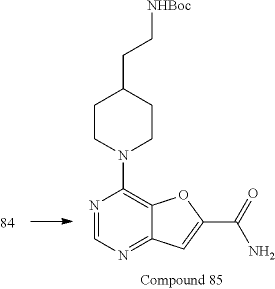 [SMILES: CC(C)(C)OC(=O)NCCC1CCN(c2ncnc3cc(C(N)=O)oc23)CC1]

A mixture of 4-chlorofuro[3,2-d]pyrimidine-6-carboxamide (84; 0.050 g, 0.254 mmol), tert-butyl (2-(piperidin-4-yl)ethyl)carbamate (0.069 g, 0.305 mmol), DIEA (0.0655 g, 0.508 mmol) in acetonitrile (2 mL) was stirred at 60° C. overnight. The reaction mixture was concentrated and the residue was purified by prep-TLC (CH₂Cl₂:MeOH=15:1) to obtain tert-butyl (2-(1-(6-carbamoylfuro[3,2-d]pyrimidin-4-yl)piperidin-4-yl)ethyl)carbamate (Compound 85; 0.040 g, 40%). MS (ESI) calcd for C₁₉H₂₇N₅O₄: 389.21. found: 390 [M+H].

Compounds 86, 87, 88, 89, 90, 91, 92, 93 and 94 of Table 7 were prepared in an analogous manner.

### Step 4. Synthesis of 4-(4-(2-aminoethyl)piperidin-1-yl)furo[3,2-d]pyrimidine-6-carboxamide (Compound 95)

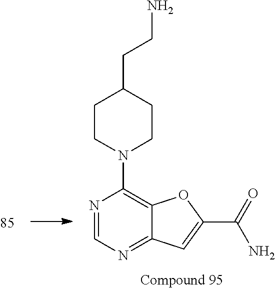 [SMILES: NCCC1CCN(c2ncnc3cc(C(N)=O)oc23)CC1]

A solution of (2-(1-(6-carbamoylfuro[3,2-d]pyrimidin-4-yl)piperidin-4-yl)ethyl)carbamate (85; 0.440 g, 1.13 mol) in 1M HCl:MeOH (10 mL) was stirred at room temperature overnight. The reaction mixture was concentrated, the residue was diluted with a NaHCO₃ solution until pH=8-9, extracted with CH₂Cl₂ (3×) and concentrated under vacuum. The residue was triturated with CH₂Cl₂:MeOH (10:1) to obtain 4-(4-(2-aminoethyl)piperidin-1-yl)furo[3,2-d]pyrimidine-6-carboxamide (Compound 95; 0.2165 g, 66%). MS (ESI) calcd for C₁₄H₁₉N₅O₂: 289.15. found: 290 [M+H].

Compound 96 of Table 7 was prepared in an analogous manner.

### Step 5. Synthesis of 4-(4-(2-pivalamidoethyl)piperidin-1-yl)furo[3,2-d]pyrimidine-6-carboxamide (Compound 36)

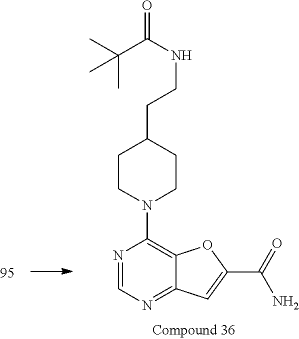 [SMILES: CC(C)(C)C(=O)NCCC1CCN(c2ncnc3cc(C(N)=O)oc23)CC1]

A solution of 4-(4-(2-aminoethyl)piperidin-1-yl)furo[3,2-d]pyrimidine-6-carboxamide (95; 0.100 g, 0.346 mmol) and pyridine (0.055 g, 0.692 mmol) in CH₂Cl₂ (6 mL) was cooled to 0° C. and pivaloyl chloride (0.083 g, 0.692 mmol) was added slowly. The reaction mixture was stirred at 0° C. for 15 min, and then at room temperature overnight. The reaction solution was quenched with ammonium hydroxide, concentrated under vacuum and the residue was purified by column chromatography (15:1 CH₂Cl₂/MeOH) to obtain 4-(4-(2-pivalamidoethyl)piperidin-1-yl)furo[3,2-d]pyrimidine-6-carboxamide (Compound 36; 0.0775 g, 60%). MS (ESI) calcd for C₁₉H₂₇N₅O₃: 373.21. found: 374 [M+H]. Compounds 97, 98 and 99 of Table 7 were prepared in an analogous manner.

### Example 30

### Preparation of 4-([4,4′-bipiperidin]-1-yl)furo[3,2-d]pyrimidine-6-carboxamide (Compound 100)

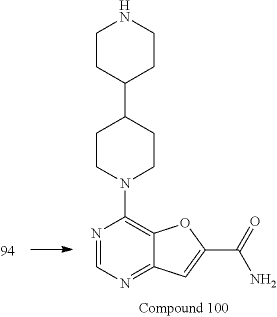 [SMILES: NC(=O)c1cc2ncnc(N3CCC(C4CCNCC4)CC3)c2o1]

A solution of tert-butyl 1′-(6-carbamoylfuro[3,2-d]pyrimidin-4-yl)-[4,4′-bipiperidine]-1-carboxylate (94; 0.044 g, 0.1 mmol) and 25% TFA in CH₂Cl₂ (4 mL) was stirred at room temperature overnight. The reaction mixture was concentrated, triturated with diethyl ether and pentane. The residue was purified by prep-HPLC and lyophilized to obtain 4-([4,4′-bipiperidin]-1-yl)furo[3,2-d]pyrimidine-6-carboxamide (Compound 100; 0.034 g, 75%). MS (ESI) calcd for C₁₇H₂₃N₅O₂: 329.19. found: 330 [M+H].

### Example 31

### Preparation of 7-(4-(2-pivalamidoethyl)piperidin-1-yl)thieno[3,2-b]pyridine-2-carboxamide (Compound 37)

### Step 1. Synthesis of 7-chlorothieno[3,2-b]pyridine-2-carboxamide (Compound 102)

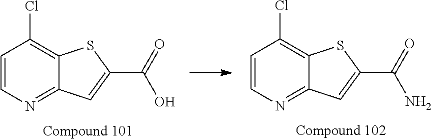 [SMILES: O=C(O)c1cc2nccc(Cl)c2s1 ; NC(=O)c1cc2nccc(Cl)c2s1]

A slurry of 7-chlorothieno[3,2-b]pyridine-2-carboxylic acid (Compound 101 from ASDI Inc., 0.64 g, 3.0 mmol), thionyl chloride (3 mL), DMF (2 drops) in CH₂Cl₂ (15 mL) was heated at reflux for 2 h. The reaction mixture was concentrated and dried under vacuum. To the residue was added dioxane (20 mL) followed by a solution of 0.5M ammonia in dioxane (30 mL, 15 mmol). The reaction mixture was stirred at room temperature for 72 h and concentrated to dryness. The residue was suspended in a mixture of EtOAc and aq. NaHCO₃ (sat.) The rag layer was removed by filtration and the organic layer was washed with brine, dried (Na₂SO₄) and concentrated to dryness to afford 7-chlorothieno[3,2-b]pyridine-2-carboxamide (Compound 102; 0.302 g, 47%). MS (ESI) calcd for C₈H₅ClN₂OS: 211.98. found: 213 [M+H].

### Step 2. Synthesis of tert-butyl (2-(1-(2-carbamoylthieno[3,2-b]pyridin-7-yl)piperidin-4-yl)ethyl)carbamate (Compound 103)

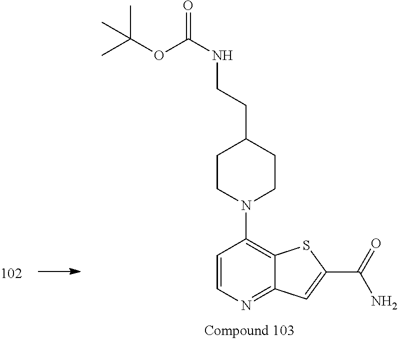 [SMILES: CC(C)(C)OC(=O)NCCC1CCN(c2ccnc3cc(C(N)=O)sc23)CC1]

A solution of 7-chlorothieno[3,2-b]pyridine-2-carboxamide (102; 0.150 g, 0.705 mmol) and tert-butyl (2-(piperidin-4-yl)ethyl)carbamate (0.242 g, 1.06 mmol) in NMP (4 mL) was microwave heated at 200° C. for 2 h. The reaction mixture was concentrated, diluted with CH₂Cl₂, filtered and concentrated. The crude tert-butyl (2-(1-(2-carbamoylthieno[3,2-b]pyridin-7-yl)piperidin-4-yl)ethyl)carbamate (Compound 103) was purified by column chromatography (0 to 10% MeOH in CH₂Cl₂) to afford Compound 103. MS (ESI) calcd for C₂₀H₂₈N₄O₃S: 404.19. found: 405 [M+H].

### Step 3. Synthesis of 7-(4-(2-aminoethyl)piperidin-1-yl)thieno[3,2-b]pyridine-2-carboxamide (Compound 104)

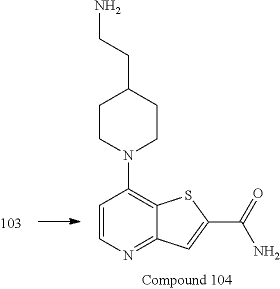 [SMILES: NCCC1CCN(c2ccnc3cc(C(N)=O)sc23)CC1]

tert-butyl (2-(1-(2-carbamoylthieno[3,2-b]pyridin-7-yl)piperidin-4-yl)ethyl)carbamate (103; assumed 0.705 mmol) was stirred in 10% TFA/CH₂Cl₂ overnight and concentrated to obtain 7-(4-(2-aminoethyl)piperidin-1-yl)thieno[3,2-b]pyridine-2-carboxamide as the 2,2,2 trifluoroacetate (Compound 104). MS (ESI) calcd for C₁₅H₂₀N₄OS: 304.14. found: 305 [M+H].

### Step 4. Synthesis of 7-(4-(2-pivalamidoethyl)piperidin-1-yl)thieno[3,2-b]pyridine-2-carboxamide (Compound 37)

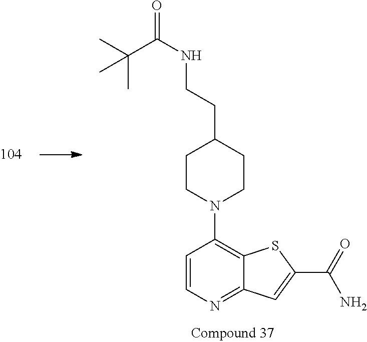 [SMILES: CC(C)(C)C(=O)NCCC1CCN(c2ccnc3cc(C(N)=O)sc23)CC1]

To 7-(4-(2-aminoethyl)piperidin-1-yl)thieno[3,2-b]pyridine-2-carboxamide (104; assumed 0.705 mmol) was added water (5 mL), ethyl acetate (10 mL), sodium carbonate (0.747 g, 7.0 mmol) followed by pivaloyl chloride (130 μl, 1.05 mmol). The reaction mixture was stirred at room temperature overnight and extracted with ethyl acetate. The organic layer was washed with water, brine, dried (Na₂SO₄) and concentrated. The product was purified by column chromatography (0 to 10% MeOH in CH₂Cl₂ gradient) (Compound 37; 0.025 g, 9%). MS (ESI) calcd for C₂₀H₂₈N₄O₂S: 388.19. found: 389 [M+H]. Compound 105 of Table 7 was prepared in an analogous manner.

### Example 32

### Preparation of 7-(4-(2-pivalamidoethyl)piperidin-1-yl)thieno[2,3-c]pyridine-2-carboxamide (Compound 38)

### Step 1. Synthesis of 4-bromo-2-(methoxycarbonyl)thieno[2,3-c]pyridine 6-oxide (Compound 107)

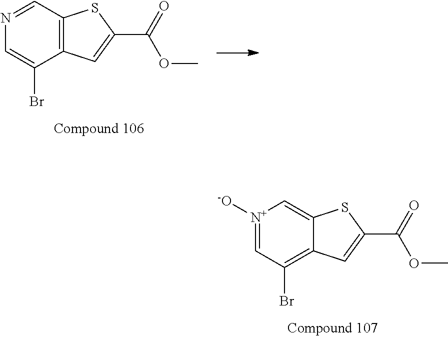

At 0° C., to a solution of methyl 4-bromothieno[2,3-c]pyridine-2-carboxylate (Compound 106; 1.9 g, 7.0 mmol) in CH₂Cl₂ (50 mL) was added meta-chloroperoxybenzoic acid (1.7 g, 8.4 mmol), and the mixture was allowed to warm to room temperature and stirred overnight. The mixture was diluted with CH₂Cl₂ (100 mL), the organic layer was washed with 1 N NaOH solution, brine and water, dried over anhydrous Na₂SO₄, filtered and concentrated to obtain crude 4-bromo-2-(methoxycarbonyl)thieno[2,3-c]pyridine 6-oxide as a solid which was used without further purification (Compound 107; 2.8 g, assumed quantitative). MS (ESI) calcd for C₉H₆BrNO₃: 286.93.

### Step 2. Synthesis of methyl 4-bromo-7-chlorothieno[2,3-c]pyridine-2-carboxylate (Compound 108)

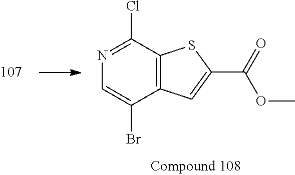

To a solution of 4-bromo-2-(methoxycarbonyl)thieno[2,3-c]pyridine 6-oxide (107; 2.0 g, 6.9 mmol) in CHCl₃ was added POCl₃ (1.95 mL, 20.8 mmol) at 0° C. under N₂ atmosphere. The reaction was then warmed and refluxed overnight. After cooling down, the mixture was concentrated under reduced pressure. The residue was purified by silica gel chromatography using petroleum ether:ethyl acetate (10:1) to provide methyl 4-bromo-7-chlorothieno[2,3-c]pyridine-2-carboxylate (Compound 108; 0.8 g, 38%). MS (ESI) calcd for C₉H₅BrClNO₂S: 304.89.

### Step 3. Synthesis of 4-bromo-7-chlorothieno[2,3-c]pyridine-2-carboxamide (Compound 109)

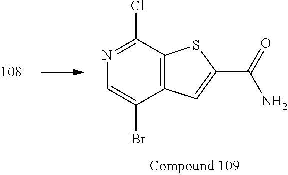 [SMILES: NC(=O)c1cc2c(Br)cnc(Cl)c2s1]

The mixture of methyl 4-bromo-7-chlorothieno[2,3-c]pyridine-2-carboxylate (108; 0.200 g, 0.65 mmol) and MeOH/NH₃ (2.0 M, 30 mL) was stirred at 45° C. overnight. After cooling down, the mixture was concentrated to afford 4-bromo-7-chlorothieno[2,3-c]pyridine-2-carboxamide (Compound 109; 0.185 g, 97%) as a white solid. MS (ESI) calcd for C₈H₄BrClN₂OS: 289.89.

### Step 4. Synthesis of tert-butyl (2-(1-(4-bromo-2-carbamoylthieno[2,3-c]pyridin-7-yl)piperidin-4-yl)ethyl)carbamate (Compound 110)

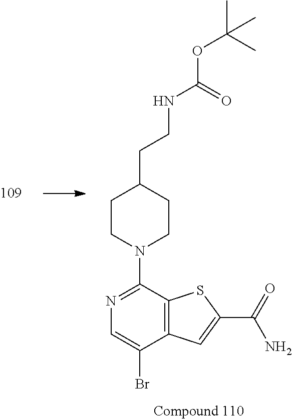 [SMILES: CC(C)(C)OC(=O)NCCC1CCN(c2ncc(Br)c3cc(C(N)=O)sc23)CC1]

To a solution of 4-bromo-7-chlorothieno[2,3-c]pyridine-2-carboxamide (109; 0.100 g, 0.34 mmol) and tert-butyl (2-(piperidin-4-yl)ethyl) carbamate (0.118 g, 0.51 mmol) in i-PrOH (10.0 mL) was added DIEA (0.5 mL). The mixture was heated to 160° C. under microwave conditions for 3 h. After cooling down, the resulting mixture was concentrated. The residue was dissolved in CH₂Cl₂ (20 mL), washed with saturated NaHCO₃, the organic phase was separated and the aqueous phase extracted with CH₂Cl₂ (3×20 mL). The combined organic layers were washed with brine, dried over anhydrous Na₂SO₄, filtered and concentrated. The resulting material was purified by preparative-TLC using 3:2 CH₂Cl₂/ethyl acetate to obtain tert-butyl(2-(1-(4-bromo-2-carbamoylthieno[2,3-c]pyridin-7-yl)piperidin-4-yl)ethyl)carbamate (Compound 110; 0.077 g, 46%) as a light yellow solid. MS (ESI) calcd for C₂₀H₂₇BrN₄O₃S: 482.10.

### Step 5. Synthesis of 7-(4-(2-aminoethyl)piperidin-1-yl)-4-bromothieno[2,3-c]pyridine-2-carboxamide (Compound 111)

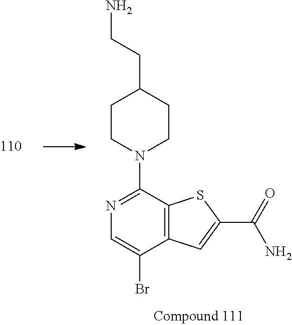 [SMILES: NCCC1CCN(c2ncc(Br)c3cc(C(N)=O)sc23)CC1]

The mixture of tert-butyl(2-(1-(4-bromo-2-carbamoylthieno[2,3-c]pyridin-7-yl)piperidin-4-yl)ethyl)carbamate (110; 0.077 g, 0.16 mmol) and MeOH/HCl (2.0 M, 5.0 mL) was stirred at room temperature overnight. After removing the solvent, the HCl salt of 7-(4-(2-aminoethyl)piperidin-1-yl)-4-bromothieno[2,3-c]pyridine-2-carboxamide (Compound 111; 0.120 g, assumed quantitative) was obtained as a yellow solid and used without further purification. MS (ESI) calcd for C₁₅H₁₉BrN₄OS: 382.05.

### Step 6. Synthesis of 4-bromo-7-(4-(2-pivalamidoethyl)piperidin-1-yl)thieno[2,3-c]pyridine-2-carboxamide (Compound 112)

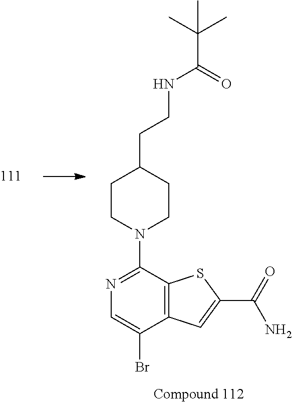 [SMILES: CC(C)(C)C(=O)NCCC1CCN(c2ncc(Br)c3cc(C(N)=O)sc23)CC1]

To a solution of 7-(4-(2-aminoethyl)piperidin-1-yl)-4-bromothieno[2,3-c]pyridine-2-carboxamide (111; 0.040 g, 0.08 mmol) and pyridine (1.8 mL, 0.24 mmol) in CH₂Cl₂ (2.0 mL) was added pivaloyl chloride (0.0193 g, 0.168 mmol) dropwise at 0° C. over 10 min. The reaction mixture was warmed to room temperature and stirred overnight. The mixture was diluted with CH₂Cl₂ (10 mL) and water (5 mL), the organic phase was separated and the aqueous phase was extracted with CH₂Cl₂ (3×10 mL). The combined organic layers were washed with saturated NaHCO₃ and brine, dried over anhydrous Na₂SO₄, filtered and concentrated. The crude was purified by recrystallization from ethyl acetate to afford 4-bromo-7-(4-(2-pivalamidoethyl)piperidin-1-yl)thieno[2,3-c]pyridine-2-carboxamide (Compound 112; 0.0176 g, 47%) as a light yellow solid. MS (ESI) calcd for C₂₀H₂₇BrN₄O₂S: 466.10. found: 467 [M+H].

### Step 7. Synthesis of 7-(4-(2-pivalamidoethyl)piperidin-1-yl)thieno[2,3-c]pyridine-2-carboxamide (Compound 38)

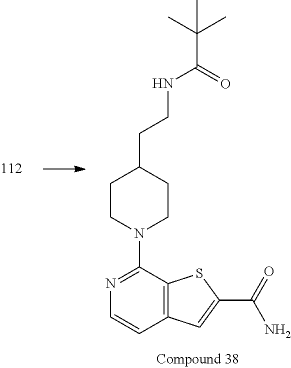 [SMILES: CC(C)(C)C(=O)NCCC1CCN(c2nccc3cc(C(N)=O)sc23)CC1]

The mixture of 4-bromo-7-(4-(2-pivalamidoethyl)piperidin-1-yl)thieno[2,3-c]pyridine-2-carboxamide (112; 0.050 g, 0.11 mmol) and Pd/C (10%, 0.015 g) in MeOH (10 mL) was stirred at room temperature under H₂ atmosphere overnight. The solid was filtered and the filtrate was concentrated. The residue was diluted with ethyl acetate (20 mL) and saturated NaHCO₃ (5 mL), the organic phase was separated and the aqueous phase were extracted with ethyl acetate (3×15 mL). The combined organic layers were washed with brine, dried over anhydrous Na₂SO₄, filtered and concentrated. The resulting material was purified by preparative-TLC using ethyl acetate to obtain 7-(4-(2-pivalamidoethyl)piperidin-1-yl)thieno[2,3-c]pyridine-2-carboxamide (Compound 38; 0.004 g, 10%) as a light yellow solid. MS (ESI) calcd for C₂₀H₂₈N₄O₂S: 388.19. found: 389 [M+H].

### Example 33

### Preparation of tert-butyl (2-(1-(6-carbamoyl-7-methylthieno[3,2-d]pyrimidin-4-yl)piperidin-4-yl)ethyl)carbamate (Compound 39)

### Step 1. Synthesis of 4-chloro-7-methylthieno[3,2-d]pyrimidine-6-carboxylic acid (Compound 114)

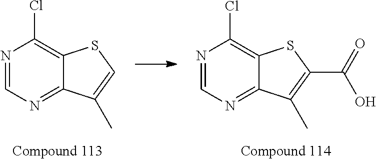

A solution of diisopropylamine (2.71 mL, 0.0193 mol) in dry THF (70 mL) was cooled to −78° C. A solution of n-butyllithium (2.5 M) in hexanes was added to this reaction mixture dropwise. After stirring for 30 minutes at −78° C., the mixture was added dropwise via syringe to a stirring suspension of 4-chloro-7-methylthieno[3,2-d]pyrimidine (Compound 113; 2.5 g, 0.0135 mol) in THF at −78° C. The suspension became homogenous when the addition was complete. The mixture was kept at −78° C. for 45 minutes and then CO₂ gas was bubbled through the reaction mixture for 10 minutes causing the green color to disappear. The reaction was kept under dry nitrogen gas and allowed to warm to room temperature overnight. The mixture was concentrated under reduced pressure and then THF was added, the mixture stirred for 1 hour and then filtered. The collected solid was suspended in dilute aqueous HCl, stirred, collected and washed with dilute aqueous HCl. The resulting beige solid was dried overnight under vacuum to obtain 4-chloro-7-methylthieno[3,2-d]pyrimidine-6-carboxylic acid (Compound 114; 2.71 g, 88%). MS (ESI) calcd for C8H5ClN₂O₂S: 227.98. found: 229 [M+H].

### Step 2. Synthesis of 4-chloro-7-methylthieno[3,2-d]pyrimidine-6-carboxamide (Compound 115)

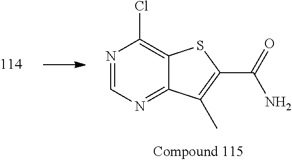

To a suspension of 4-chloro-7-methylthieno[3,2-d]pyrimidine-6-carboxylic acid (114; 0.518 g, 2.27 mmol) in CH₂Cl₂ (20 mL) was added oxalyl chloride (395 μL, 4.53 mmol). A small amount of DMF (50 μL) was added dropwise which resulted in gas evolution from the mixture. The reaction was stirred at room temperature overnight during which it became nearly homogeneous. The reaction was heated at 60° C. for 2.5 h then cooled to room temperature. Additional oxalyl chloride (500 μL) was added and the mixture was heated at 60° C. for 3 h, cooled to room temperature and concentrated under reduced pressure. Dichloromethane was added, the mixture was brought to 0° C., and dry NH₃ gas was bubbled through the solution for 5 minutes. After 1 h, the solution was diluted with additional CH₂Cl₂ and then washed with saturated NaHCO₃ solution (2×). The solid, which did not dissolve in either layer, was collected by filtration and dried to afford 4-chloro-7-methylthieno[3,2-d]pyrimidine-6-carboxamide (Compound 115; 0.200 g, 39%) as a beige solid. MS (ESI) calcd for C₈H₆ClN₃OS: 226.99. found: 228.0 [M+H].

### Step 3. Synthesis of tert-butyl (2-(1-(6-carbamoyl-7-methylthieno[3,2-d]pyrimidin-4-yl)piperidin-4-yl)ethyl)carbamate (Compound 116)

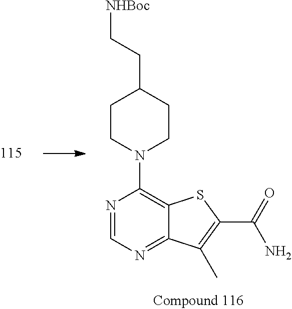 [SMILES: Cc1c(C(N)=O)sc2c(N3CCC(CCNC(=O)OC(C)(C)C)CC3)ncnc12]

A mixture of 4-chloro-7-methylthieno[3,2-d]pyrimidine-6-carboxamide (115; 0.195 g, 0.857 mmol), tert-butyl (2-(piperidin-4-yl)ethyl)carbamate (259 mg, 1.13 mmol), diisopropylethylamine (223 mL, 1.29 mmol) and acetonitrile was heated at 60° C. for 2 h. Additional tert-butyl (2-(piperidin-4-yl)ethyl)carbamate (0.050 g) was added and heating was continued at 60° C. for 16 h. The mixture was cooled, diluted with ethyl acetate and washed with 5% aqueous HCl. The ethyl acetate layer was dried (Na₂SO₄) and concentrated to give a solid. The acidic extract was basified with 10% NaOH solution to give a milky solution that was extracted with ethyl acetate, dried (Na₂SO₄) and concentrated. The material furnished from ethyl acetate extracts from both the acidic and basic solutions were combined and then purified on a 12 g silica gel cartridge eluting with 50% pentane/ethyl acetate to 100% ethyl acetate to afford tert-butyl (2-(1-(6-carbamoyl-7-methylthieno[3,2-d]pyrimidin-4-yl)piperidin-4-yl)ethyl)carbamate (Compound 116; 0.285 g, 79%) as a beige foam. MS (ESI) calcd for C₂₀H₂₉N₅O₃S: 419.20. found: 420 [M+H].

### Step 4. Synthesis of 4-(4-(2-aminoethyl)piperidin-1-yl)-7-methylthieno[3,2-d]pyrimidine-6-carboxamide (Compound 117)

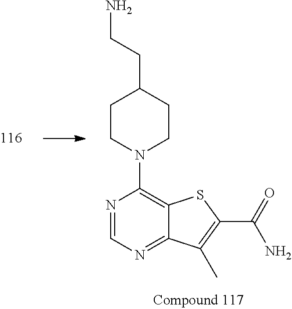 [SMILES: Cc1c(C(N)=O)sc2c(N3CCC(CCN)CC3)ncnc12]

A solution of tert-butyl (2-(1-(6-carbamoyl-7-methylthieno[3,2-d]pyrimidin-4-yl)piperidin-4-yl)ethyl)carbamate (116; 0.265 g, 0.633 mmol) was treated with trifluoroacetic acid (487 μL, 6.33 mmol) and the resulting solution stirred at room temperature for 16 h. An additional 200 μL of trifluoroacetic acid was added and the mixture heated at 40° C. for 1 h. The biphasic reaction mixture was concentrated under reduced pressure and the residue dissolved in ethyl acetate. Pentane was added until the solution was slightly cloudy and the mixture was allowed to stand at room temperature. The resulting precipitate was collected via filtration and washed with 1:1 ethyl acetate/pentane (3×). The solid was dried under vacuum to afford 4-(4-(2-aminoethyl)piperidin-1-yl)-7-methylthieno[3,2-d]pyrimidine-6-carboxamide mono triflate salt (Compound 117; 0.266 g, 97%) as a cream colored solid. MS (ESI) calcd for C₁₅H₂₁N₅OS: 319.15. found: 320 [M+H].

### Step 5. Synthesis of 7-methyl-4-(4-(2-pivalamidoethyl)piperidin-1-yl)thieno[3,2-d]pyrimidine-6-carboxamide (Compound 39)

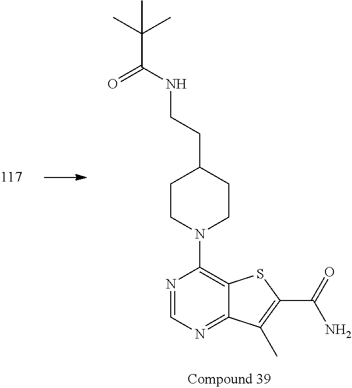 [SMILES: Cc1c(C(N)=O)sc2c(N3CCC(CCNC(=O)C(C)(C)C)CC3)ncnc12]

A solution of 4-(4-(2-aminoethyl)piperidin-1-yl)-7-methylthieno[3,2-d]pyrimidine-6-carboxamide mono triflate salt (117; 0.109 g, 0.251 mmol) in 1:1 Na₂CO₃ (1 M):ethyl acetate was stirred vigorously and pivaloyl chloride (62 mL, 0.50 mmol) was added in one portion. The mixture was stirred overnight at room temperature and then concentrated under reduced pressure. A minimal amount of water was added and the solid was collected by filtration and washed twice with water. The solid was dissolved in dichloromethane/methanol, the solution dried (Na₂SO₄), filtered and concentrated under reduced pressure to afford 7-methyl-4-(4-(2-pivalamidoethyl)piperidin-1-yl)thieno[3,2-d]pyrimidine-6-carboxamide (Compound 39; 0.071 g, 70%) as a white solid. MS (ESI) calcd for C₂₀H₂₉N₅O₂S: 403.20. found: 404 [M+H].

### Example 34

### Preparation of (3-((2-(4-(6-carbamoylthieno[3,2-d]pyrimidin-4-yl)piperazin-1-yl)ethyl)carbamoyl)phenyl)phosphonic acid (Compound 122)

### Step 1. Synthesis of ethyl 3-((bis(benzyloxy)phosphoryl)oxy)benzoate (Compound 119)

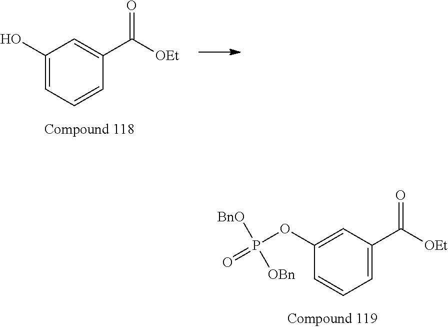 [SMILES: CCOC(=O)c1cccc(O)c1 ; CCOC(=O)c1cccc(OP(=O)(OCc2ccccc2)OCc2ccccc2)c1]

To a solution of Ethyl 3-hydroxybenzoate (Compound 118; 1.76 g, 10.6 mmol) in CH₃CN at −10° C. under nitrogen was added CCl₄ (5.12 mL, 53 mmol) followed by DIPEA (3.86 mL, 22.3 mmol) and DMAP (0.130 g, 1.1 mmol). The reaction mixture was stirred at −10° C. for 2 min, the dibenzyl phosphonate (70% tech grade, 3.4 mL) was added dropwise over 3 minutes and the reaction mixture was stirred at −10° C. for 1 hour. The reaction mixture was quenched with 0.5M KH₂PO₄ (200 mL) and extracted with ethyl acetate. The organic layer was washed with water, brine, dried over Na₂SO₄ and concentrated to dryness. The oil was purified by silica gel chromatography (0 to 40% EtOAc gradient in pentane) to obtain ethyl 3-((bis(benzyloxy)phosphoryl)oxy)benzoate (Compound 119; 4.05 g, 90%) as a clear oil. MS (ESI) calcd for C₂₃H₂₃O₆P: 426.12. found: 427 [M+H].

### Step 2. Synthesis of 3-((bis(benzyloxy)phosphoryl)oxy)benzoic acid (Compound 120)

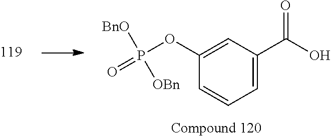 [SMILES: O=C(O)c1cccc(OP(=O)(OCc2ccccc2)OCc2ccccc2)c1]

Ethyl 3-((bis(benzyloxy)phosphoryl)oxy)benzoate (119; 4.05 g, 9.5 mmol) was dissolved in THF (50 mL) and a solution of LiOH (0.227 g, 9.50 mmol) in water (10 mL) was added. A second equivalent of LiOH (0.227 g, 9.50 mmol) was added and the reaction mixture showed some decomposition. The reaction mixture was concentrated to dryness, and acidified to pH=3 with aqueous HCl. The reaction mixture was extracted with ethyl acetate, and the organic layer was washed with brine, dried over Na₂SO₄ and concentrated to an oil. The oil was purified by Prep HPLC. The fractions were concentrated to remove the acetonitrile, and acidified to pH=1-2 with conc. HCl. The product was extracted with ethyl acetate, washed with brine, dried over Na₂SO₄ and concentrated to obtain the 3-((bis(benzyloxy)phosphoryl)oxy)benzoic acid (Compound 120; 1.00 g, 26%). MS (ESI) calcd for C₂₁H₁₉O₆P: 398.09. found: 399 [M+H].

### Step 3. Synthesis of dibenzyl (3-((2-(4-(6-carbamoylthieno[3,2-d]pyrimidin-4-yl)piperazin-1-yl)ethyl)carbamoyl)phenyl)phosphate (Compound 121)

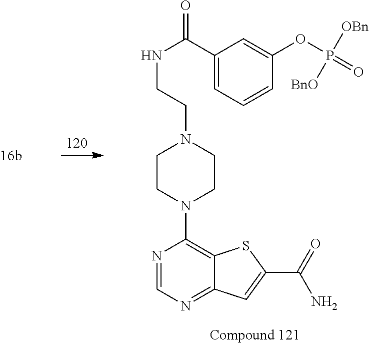 [SMILES: NC(=O)c1cc2ncnc(N3CCN(CCNC(=O)c4cccc(OP(=O)(OCc5ccccc5)OCc5ccccc5)c4)CC3)c2s1]

A solution of 4-(4-(2-aminoethyl)piperazin-1-yl)thieno[3,2-d]pyrimidine-6-carboxamide dihydrochloride (16b; 0.250 g, 0.659 mmol), HATU (0.376 g, 0.989 mmol), DIEPA (228 μL, 1.31 mmol) in DMF (3 mL) was stirred for 5 min, then a slurry of 3-((bis(benzyloxy)phosphoryl)oxy)benzoic acid (120; 0.394 g, 0.989 mmol) DIEPA (228 μL, 1.31 mmol) in DMF (3 mL) was added. The reaction mixture was stirred at room temperature for 5 hours, diluted with CH₂Cl₂, washed with sat. NaHCO₃, water, brine, dried (MgSO₄) and concentrated to red oil. The product was purified by silica gel chromatography (0 to 10% MeOH gradient in CH₂Cl₂) to obtain dibenzyl (3-((2-(4-(6-carbamoylthieno[3,2-d]pyrimidin-4-yl)piperazin-1-yl)ethyl)carbamoyl)phenyl) phosphate (Compound 121; 0.169 g, 37%). MS (ESI) calcd for C₃₄H₃₅N₆O₆PS: 686.21. found: 687 [M+H].

### Step 4. Synthesis of (3-((2-(4-(6-carbamoylthieno[3,2-d]pyrimidin-4-yl)piperazin-1-yl)ethyl)carbamoyl)phenyl)phosphonic acid (Compound 122)

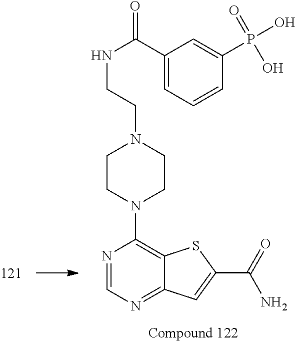 [SMILES: NC(=O)c1cc2ncnc(N3CCN(CCNC(=O)c4cccc(P(=O)(O)O)c4)CC3)c2s1]

To dibenzyl (3-((2-(4-(6-carbamoylthieno[3,2-d]pyrimidin-4-yl)piperazin-1-yl)ethyl)carbamoyl)phenyl) phosphate (121; 0.169 g, 0.25 mmol) was added 10% Pd/C (0.015 g), formic acid (15 mL) and the reaction mixture was stirred over hydrogen (1 atm) overnight. The reaction mixture was filtered through celite, the cake was washed with formic acid and 10% Pd/C (0.015 g) was charged to the mother liquor and stirred over hydrogen (1 atm) over the weekend. The reaction mixture was filtered through celite, concentrated to dryness, triturated with a mixture of diethyl ether/pentane and triturated with MeOH. The solid was collected by filtration and dried under vacuum to obtain (3-((2-(4-(6-carbamoylthieno[3,2-d]pyrimidin-4-yl)piperazin-1-yl)ethyl)carbamoyl)phenyl)phosphonic acid (Compound 122; 0.063 mg, 50% yield) as a white solid. MS (ESI) calcd for C₂₀H₂₃N₆O₅PS: 490.12. found: 491 [M+H].

### Example 35

### Preparation of N1-(2-aminoethyl)-N3-(2-(4-(6-carbamoylthieno[3,2-d]pyrimidin-4-yl)piperazin-1-yl)ethyl)isophthalamide (Compound 127)

### Step 1. Synthesis of methyl 3-((2-((tert-butoxycarbonyl)amino)ethyl)carbamoyl)benzoate (Compound 124)

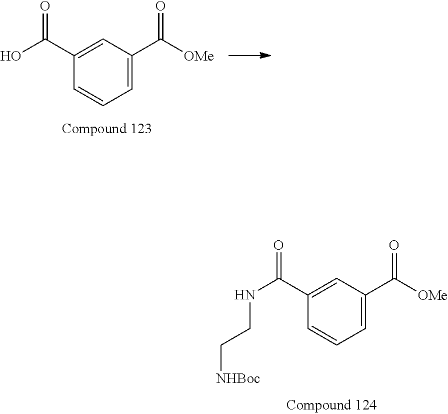 [SMILES: COC(=O)c1cccc(C(=O)O)c1 ; COC(=O)c1cccc(C(=O)NCCNC(=O)OC(C)(C)C)c1]

A solution of 3-(methoxycarbonyl)benzoic acid (Compound 123; 1.0 g, 5.55 mmol), HATU (2.53 g, 6.7 mmol) and DIEPA (1.44 mL, 8.31 mmol) I nDMF (15 mL) was stirred at room temperature for 10 minutes, then a solution of tert-butyl(2-aminoethyl)carbamate (1.07 g, 6.68 mmol) in DMF (4 mL) was added and the reaction mixture was stirred at room temperature for 2.5 hours. The reaction mixture was diluted with ethyl acetate (150 mL) and the organic layer was washed with sat. NaHCO₃, water (2×), brine, dried over Na₂SO₄ and concentrated to obtain methyl 3-((2-((tert-butoxycarbonyl)amino)ethyl)carbamoyl)benzoate (Compound 124; 1.69 g, 94%) as a red solid. MS (ESI) calcd for C₁₆H₂₂N₂O₅: 322.15.

### Step 2. Synthesis of 3-((2-((tert-butoxycarbonyl)amino)ethyl)carbamoyl)benzoic acid (Compound 125)

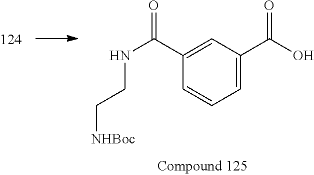 [SMILES: CC(C)(C)OC(=O)NCCNC(=O)c1cccc(C(=O)O)c1]

methyl 3-((2-((tert-butoxycarbonyl)amino)ethyl)carbamoyl)benzoate (124; 1.69 g, 5.24 mmol) was chased with methanol (2×), dissolved in methanol (50 mL) and stirred with LiOH (0.399 g, 16.7 mmol) and water (10 mL) overnight. The reaction mixture was concentrated, dissolved in water and acidified with conc. HCl to pH=4 to 5. The precipitate was collected by filtration, washed with water and dried to obtain 3-((2-((tert-butoxycarbonyl)amino)ethyl)carbamoyl)benzoic acid (Compound 125; 1.44 g, 84% yield) as a tan solid. MS (ESI) calcd for C₁₅H₂₀N₂O₅: 308.14.

### Step 3. Synthesis of tert-butyl (2-(3-((2-(4-(6-carbamoylthieno[3,2-d]pyrimidin-4-yl)piperazin-1-yl)ethyl)carbamoyl)benzamido)ethyl)carbamate (Compound 126)

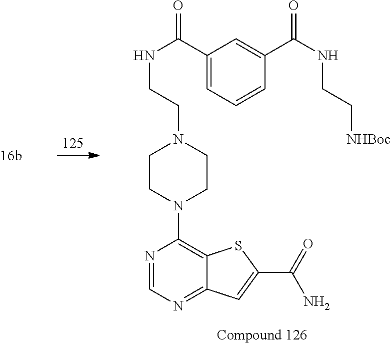 [SMILES: CC(C)(C)OC(=O)NCCNC(=O)c1cccc(C(=O)NCCN2CCN(c3ncnc4cc(C(N)=O)sc34)CC2)c1]

A solution of 4-(4-(2-aminoethyl)piperazin-1-yl)thieno[3,2-d]pyrimidine-6-carboxamide dihydrochloride (16b; 0.190 g, 0.50 mmol), 3-((2-((tert-butoxycarbonyl)amino)ethyl)carbamoyl)benzoic acid (125; 0.185 g, 0.60 mmol), HATU (0.228 g, 0.60 mmol) and DIEPA (520 μL, 3.0 mmol) in DMF (10 mL) was stirred at room temperature overnight. The reaction mixture was diluted with a 1:1 mixture of sat. aq. NaHCO₃/water and extracted with ethyl acetate. The organic layer was washed with water (2×), brine, dried over Na₂SO₄ and concentrated. The material was purified by silica gel column chromatography (10% MeOH in CH₂Cl₂) to obtain tert-butyl (2-(3-((2-(4-(6-carbamoylthieno[3,2-d]pyrimidin-4-yl)piperazin-1-yl)ethyl)carbamoyl)benzamido)ethyl)carbamate (Compound 126; 180 mg, 60%). MS (ESI) calcd for C₂₈H₃₆N₈O₅S: 596.25. found: 597 [M+H].

### Step 4. Synthesis of N1-(2-aminoethyl)-N3-(2-(4-(6-carbamoylthieno[3,2-d]pyrimidin-4-yl)piperazin-1-yl)ethyl)isophthalamide (Compound 127)

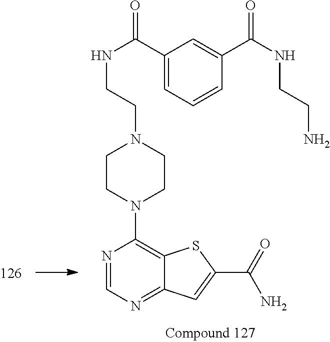 [SMILES: NCCNC(=O)c1cccc(C(=O)NCCN2CCN(c3ncnc4cc(C(N)=O)sc34)CC2)c1]

tert-butyl (2-(3-((2-(4-(6-carbamoylthieno[3,2-d]pyrimidin-4-yl)piperazin-1-yl)ethyl)carbamoyl)benzamido)ethyl)carbamate (Compound 126; 180 mg, 0.30 mmol) was diluted in CH₂Cl₂ (20 mL) and TFA (4 mL) was added. The reaction mixture was stirred at room temperature overnight, concentrated to dryness and triturated with diethyl ether/pentane and dried under vacuum to obtain N¹-(2-aminoethyl)-N³-(2-(4-(6-carbamoylthieno[3,2-d]pyrimidin-4-yl)piperazin-1-yl)ethyl)isophthalamide bis(2,2,2-trifluoroacetate) as a tan solid (Compound 127; 0.104 g, 48% yield). MS (ESI) calcd for C₂₃H₂₈N₈O₃S: 496.20. found: 497 [M+H].

Compound 191 of Table 8 was prepared in an analogous manner.

### Example 36

### Preparation of 4-(4-((3-(trifluoromethyl)piperidin-1-yl)methyl)piperidin-1-yl)thieno[3,2-d]pyrimidine-6-carboxamide (Compound 131)

### Step 1. Synthesis of tert-butyl 4-((3-(trifluoromethyl)piperidin-1-yl)methyl)piperidine-1-carboxylate (Compound 129)

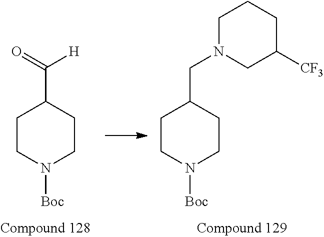 [SMILES: CC(C)(C)OC(=O)N1CCC(C=O)CC1 ; CC(C)(C)OC(=O)N1CCC(CN2CCCC(C(F)(F)F)C2)CC1]

To a solution of tert-butyl 4-formylpiperidine-1-carboxylate (Compound 128; 0.107 mg, 0.5 mmol) and 3-(trifluoromethyl)piperidine (0.153 g, 1 mmol) in CH₂Cl₂ (5 mL) was added acetic acid (2 drops). The reaction mixture was stirred for 45 min, Na(OAc)₃BH (0.159 g, 0.75 mmol) was added and the resulting solution was stirred at room temperature overnight. The reaction mixture was quenched with aqueous NaHCO₃ (sat), and the organic later was washed with sat. NaHCO₃, brine, dried (Na₂SO₄) and concentrated to obtain tert-butyl 4-((3-(trifluoromethyl)piperidin-1-yl)methyl)piperidine-1-carboxylate (Compound 129; assumed quantitative), which was used in the next step without further purification. MS (ESI) calcd for C₁₇H₂₉F₃N₂O₂:350.22.

### Step 2. Synthesis of 1-(piperidin-4-ylmethyl)-3-(trifluoromethyl)piperidine (Compound 130)

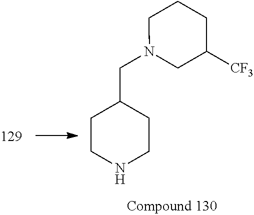 [SMILES: FC(F)(F)C1CCCN(CC2CCNCC2)C1]

tert-butyl 4-((3-(trifluoromethyl)piperidin-1-yl)methyl)piperidine-1-carboxylate (129; assumed 0.5 mmol) was stirred in 25% TFA in CH₂Cl₂ (4 mL) for 4 hours and concentrated to dryness to obtain 1-(piperidin-4-ylmethyl)-3-(trifluoromethyl)piperidine (Compound 130; assumed quantitative), which was used in the next step without further purification. MS (ESI) calcd for C₁₂H₂₁F₃N₂: 250.17.

### Step 3. Synthesis of 4-(4-((3-(trifluoromethyl)piperidin-1-yl)methyl)piperidin-1-yl)thieno[3,2-d]pyrimidine-6-carboxamide (Compound 131)

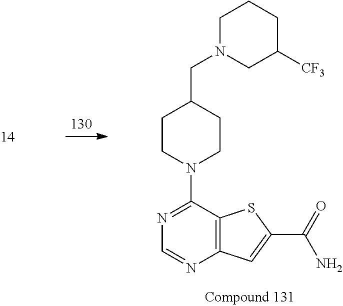 [SMILES: NC(=O)c1cc2ncnc(N3CCC(CN4CCCC(C(F)(F)F)C4)CC3)c2s1]

1-(Piperidin-4-ylmethyl)-3-(trifluoromethyl)piperidine (130; assumed 0.5 mmol), 4-chlorothieno[3,2-d]pyrimidine-6-carboxamide (14; 0.071 g, 0.333 mmol) and DIEA (230 μL, 1.33 mmol) in CH₃CN (5 mL) was heated at 85° C. for 3 days and concentrated to dryness. The residue was purified by prep-HPLC to obtain 4-(4-((3-(trifluoromethyl)piperidin-1-yl)methyl)piperidin-1-yl)thieno[3,2-d]pyrimidine-6-carboxamide as the bis-TFA salt (Compound 131; 0.051 g, 8%). MS (ESI) calcd for C₁₉H₂₄F₃N₅OS: 427.17. found: 428 [M+H].

Compound 132 of Table 7 was prepared in an analogous manner.

### Example 37

### Preparation of 5-bromo-N¹-(2-(1-(6-carbamoylthieno[3,2-d]pyrimidin-4-yl)piperidin-4-yl)ethyl)-N³-ethylisophthalamide (Compound 136)

### Step 1. Synthesis of methyl 3-bromo-5-(ethylcarbamoyl)benzoate (Compound 134)

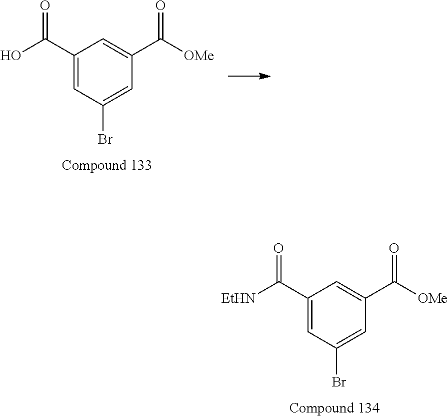

Prepared according to the published procedure of Choi K., et al., J. Am. Chem. Soc., 2003, 125 (34), pp 10241-10249). A solution of 3-bromo-5-(methoxycarbonyl)benzoic acid (Compound 133; 0.400 g, 1.54 mmol) and HATU (0.587 g, 1.54 mmol) in DMF was treated with diisopropylethylamine (538 μL, 3.09 mmol) followed by ethylamine (1.5 mL of 2M solution in THF, 3.09 mmol). The solution was stirred for two days at room temperature. The mixture was diluted with ethyl acetate, washed twice with 1N HCl solution, brine, twice with 10% aqueous NaOH solution and brine. The organic layer was dried (Na₂SO₄) and concentrated under reduced pressure to give methyl 3-bromo-5-(ethylcarbamoyl)benzoate (Compound 134; assumed quantitative). MS (ESI) calcd for C₁₁H₁₂BrNO₃:285. found: 286[M+H].

### Step 2. Synthesis of 3-bromo-5-(ethylcarbamoyl)benzoic acid (Compound 135)

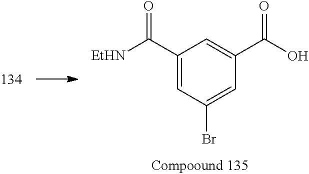

Methyl 3-bromo-5-(ethylcarbamoyl)benzoate (134; 0.452 g, 1.58 mmol) was dissolved in THF and water was added dropwise until the reaction mixture just started to become cloudy. Solid LiOH (0.303 g, 12.6 mmol) was added. A small amount of methanol was added to the stirring solution in order to increase the homogeneity of the mixture. After stirring for 3 to 4 hours, the mixture was concentrated under reduced pressure and water was added. The aqueous solution was washed twice with ether and the ether was discarded. The aqueous layer was acidified with 3N HCl to achieve a white precipitate. The mixture was extracted with ethyl acetate, dried (Na₂SO₄), and concentrated under reduced pressure to afford 3-bromo-5-(ethylcarbamoyl)benzoic acid (Compound 135; 0.302 g, 70%) as a white solid. MS (ESI) calcd for C₁₀H₁₀BrNO₃: 270.98. found: 272[M+H].

### Step 3. Synthesis of 5-bromo-N¹-(2-(1-(6-carbamoylthieno[3,2-d]pyrimidin-4-yl)piperidin-4-yl)ethyl)-N³-ethylisophthalamide (Compound 136)

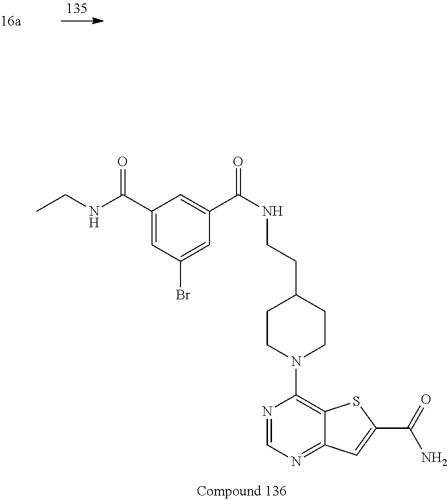 [SMILES: CCNC(=O)c1cc(Br)cc(C(=O)NCCC2CCN(c3ncnc4cc(C(N)=O)sc34)CC2)c1]

To a solution of 3-bromo-5-(ethylcarbamoyl)benzoic acid (135; 0.115 g, 0.423 mmol) in DMF was added HATU (0.161 g, 0.423 mmol) followed by diisopropylethylamine (340 μL, 1.95 mmol). Following addition of 4-(4-(2-aminoethyl)piperidin-1-yl)thieno[3,2-d]pyrimidine-6-carboxamide trifluoroacetate salt (16a; 0.136 g, 0.325 mmol), the reaction was stirred for 16 h at room temperature. The mixture was diluted with ethyl acetate and washed with 30% AcOH (2×) and then with 10% NaOH (3×). The mixture was dried (Na₂SO₄) and concentrated under reduced pressure. The product was purified on a silica gel cartridge (12 g) eluting with methanol/dichloromethane (5% to 30%) to afford 5-bromo-N¹-(2-(1-(6-carbamoylthieno[3,2-d]pyrimidin-4-yl)piperidin-4-yl)ethyl)-N³-ethylisophthalamide (Compound 136; 0.076 g, 32%). MS (ESI) calcd for C₂₄H₂₇BrN₆O₃S: 558.10. found: 559.2 [M+H].

### Example 39

### Preparation of 2-methyl-4-(4-(2-(methylsulfonamido)ethyl)piperidin-1-yl)thieno[3,2-d]pyrimidine-6-carboxamide (Compound 154)

### Step 1. Synthesis of 3-acetamidothiophene-2-carboxamide (Compound 138)

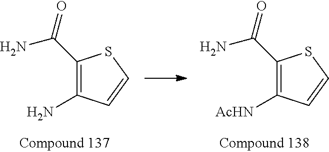 [SMILES: NC(=O)c1sccc1N ; CC(=O)Nc1ccsc1C(N)=O]

To a solution of 3-aminothiophene-2-carboxamide (Compound 137; 11.8 g, 83.1 mmol) and triethylamine (35 mL) in toluene (250 mL) was added acetic anhydride (9.5 mL) at room temperature. The reaction mixture was refluxed for 2 h. After cooling down, the solvent was removed in vacuo and the residue was washed with 1:1 petroleum ether/ethyl acetate to obtain 3-acetamidothiophene-2-carboxamide (Compound 138; 15.3 g, 100%) as a white solid. MS (ESI) calcd for C₇H₈N₂O₂S:184.03.

### Step 2. Synthesis of 2-methylthieno[3,2-d]pyrimidin-4-ol (Compound 139)

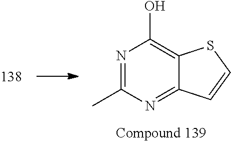

A solution of 3-acetamidothiophene-2-carboxamide (138; 15.3 g, 83 mmol) and NaOH (16.63 g, 415 mmol) in water (400 mL) was heated at reflux for 2 h. After cooling down, the resulting solution was neutralized to pH 6 using 2 N HCl at 0° C. The precipitate was collected by filtration, washed with water, and dried in vacuo to afford 2-methylthieno[3,2-d]pyrimidin-4-ol (Compound 139; 12.5 g, 91%), which was used in the next step without further purification. MS (ESI) calcd for C₇H₆N₂OS:166.02.

### Step 3. Synthesis of 4-chloro-2-methylthieno[3,2-d]pyrimidine (Compound 140)

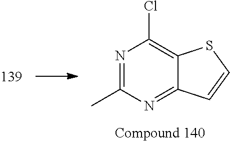

To a solution of 2-methylthieno[3,2-d]pyrimidin-4-ol (139; 10.5 g, 63.3 mmol) in DMF (12.2 mL) and 1,2-dichloroethane (250 mL) was added POCl₃ (17.6 mL) dropwise at 0° C. The reaction mixture was then heated at 150° C. for 2 h. After cooling down, the mixture was concentrated in vacuo. The residue was neutralized to pH 7 using 2N NaOH. The resulting mixture was diluted with ethyl acetate (150 mL) and water (70 mL). The organic layer was separated, and the aqueous phases were extracted with ethyl acetate (3×100 mL). The combined organic layers were washed with brine, dried over anhydrous Na₂SO₄, filtered and concentrated. The residue was purified by silica gel column chromatography using 4:1 petroleum ether/ethyl acetate to obtain crude 4-chloro-2-methylthieno[3,2-d]pyrimidine (Compound 140; 11.2 g, 97%). MS (ESI) calcd for C₇H₅ClN₂S: 183.99.

### Step 4. Synthesis of 4-methoxy-2-methylthieno[3,2-d]pyrimidine (Compound 141)

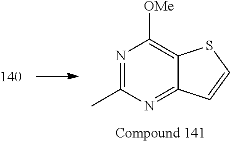

A solution of 4-chloro-2-methylthieno[3,2-d]pyrimidine (140; 11.2 g, 65.8 mmol) and sodium methoxide (30 g, 50% in MeOH) in MeOH (250 mL) was refluxed for 2 h. After cooling down, the mixture was concentrated. The residue was diluted with ethyl acetate (500 mL) and water (100 mL). The organic layer was separated, and the aqueous phases were extracted with ethyl acetate (3×100 mL), the combined organic layers were washed with brine, dried over anhydrous Na₂SO₄, filtered and concentrated. The residue was purified by silica gel column chromatography using 4:1 petroleum ether/ethyl acetate to obtain 4-methoxy-2-methylthieno[3,2-d]pyrimidine (Compound 141; 8.5 g, 72%). MS (ESI) calcd for C₈H₈N₂OS: 180.04.

### Step 5. Synthesis of 4-methoxy-2-methylthieno[3,2-d]pyrimidine-6-carboxylic acid (Compound 142)

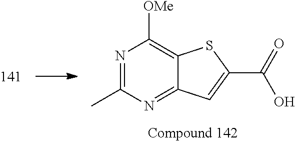

To the solution of 4-methoxy-2-methylthieno[3,2-d]pyrimidine (141; 0.5 g, 2.78 mmol) in THF (25 mL) was added n-BuLi (1.5 ml, 3.7 mmol) at −78° C. under N₂ atmosphere. The mixture was stirred at this temperature for 1 h. Dry CO₂ was bubbled in and the mixture was warmed to −20° C. and stirred for 3 h. The reaction was quenched with sat. NH₄Cl, neutralized to pH 3 using 2N HCl. The precipitate was collected, washed with water, and dried in vacuo to obtain 4-methoxy-2-methylthieno[3,2-d]pyrimidine-6-carboxylic acid (Compound 142; 0.5 g, 80%) which was used in the next step without further purification. MS (ESI) calcd for C₉H₈N₂O₃S: 224.03.

### Step 6. Synthesis 4-hydroxy-2-methylthieno[3,2-d]pyrimidine-6-carboxylic acid (Compound 143)

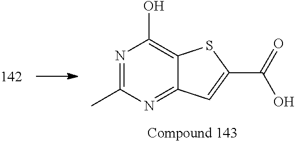

A mixture of 4-methoxy-2-methylthieno[3,2-d]pyrimidine-6-carboxylic acid (142; 0.150 g, 0.669 mmol) and 6 N HCl (2 mL) was stirred at 100° C. for 1.5 h. After cooling down, the solvent was removed in vacuo, to afford crude 4-hydroxy-2-methylthieno[3,2-d]pyrimidine-6-carboxylic acid (Compound 143; 0.150 g, quantitative). MS (ESI) calcd for C₈H₆N₂O₃S: 210.01.

### Step 7. Synthesis of 4-chloro-2-methylthieno[3,2-d]pyrimidine-6-carboxamide (Compound 144)

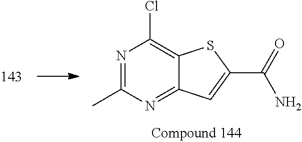

To a solution of 4-hydroxy-2-methylthieno[3,2-d]pyrimidine-6-carboxylic acid (143; 0.150 g, 0.72 mmol) in DMF (0.14 mL) and 1,2-dichloroethane (25 mL) was added POCl₃ (0.2 mL) dropwise at 0° C. The reaction mixture was then heated at 150° C. for 2 h. After cooling down, the mixture was concentrated in vacuo. The residue was neutralized with 2 N NH₃ in dioxane at 0° C. and the mixture was stirred for an additional 3 h. The solvent was then removed again and the residue was purified by silica gel column chromatography using 1:1 petroleum ether/ethyl acetate to obtain 4-chloro-2-methylthieno[3,2-d]pyrimidine-6-carboxamide (Compound 144; 0.025 g, 15%) as a white solid. MS (ESI) calcd for C₈H₆ClN₃OS: 226.99.

### Step 8. Synthesis of tert-butyl (2-(1-(6-carbamoyl-2-methylthieno[3,2-d]pyrimidin-4-yl)piperidin-4-yl)ethyl)carbamate (Compound 145)

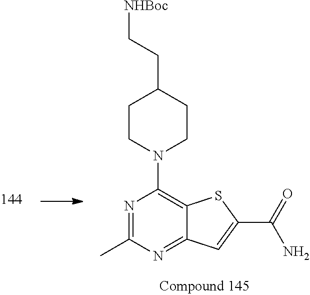 [SMILES: Cc1nc(N2CCC(CCNC(=O)OC(C)(C)C)CC2)c2sc(C(N)=O)cc2n1]

A mixture of 4-chloro-2-methylthieno[3,2-d]pyrimidine-6-carboxamide (144; 0.200 g, 0.88 mmol), tert-butyl (2-(piperidin-4-yl)ethyl)carbamate (0.241 g, 1.06 mmol) and DIEA (0.5 mL) in acetonitrile (5 mL) was heated at 60° C. for 24 h. After cooling down, the solvent was removed, and residue was purified by prep-TLC (1:10 MeOH in CH₂Cl₂) to obtain tert-butyl (2-(1-(6-carbamoyl-2-methylthieno[3,2-d]pyrimidin-4-yl)piperidin-4-yl)ethyl)carbamate (Compound 145; 0.150 g, 41%). MS (ESI) calcd for C₂₀H₂₉N₅O₃S: 419.20. found: 420 [M+H].

Compounds 146, 147, 148, 149, 150 and 151 of Table 7 were prepared in an analogous manner.

### Step 9. Synthesis of 4-(4-(2-aminoethyl)piperidin-1-yl)-2-methylthieno[3,2-d]pyrimidine-6-carboxamide (Compound 152)

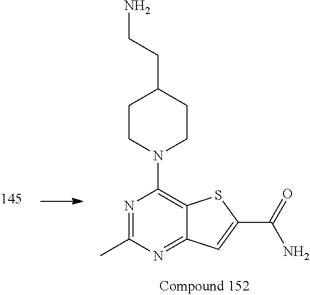 [SMILES: Cc1nc(N2CCC(CCN)CC2)c2sc(C(N)=O)cc2n1]

A mixture of tert-butyl (2-(1-(6-carbamoyl-2-methylthieno[3,2-d]pyrimidin-4-yl)piperidin-4-yl)ethyl)carbamate (145; 0.310 g, 0.74 mmol) in HCl/MeOH (5 mL) was stirred at room temperature for 4 h. The solvent was removed. The residue was neutralized to pH 7 using sat. Na₂CO₃. The mixture was extracted with CH₂Cl₂ (3×15 mL). The combined organic layers were washed with brine, dried and concentrated. The residue was purified by prep-TLC (1:10 MeOH in CH₂Cl₂) to obtain 4-(4-(2-aminoethyl)piperidin-1-yl)-2-methylthieno[3,2-d]pyrimidine-6-carboxamide (Compound 152; 0.160 g, 67%) as a white solid. MS (ESI) calcd for C₁₅H₂₁N₅OS: 319.15. found: 420 [M+H].

Compound 153 of Table 7 was prepared in an analogous manner.

### Step 10. Synthesis of 2-methyl-4-(4-(2-(methylsulfonamido)ethyl)piperidin-1-yl)thieno[3,2-d]pyrimidine-6-carboxamide (Compound 154)

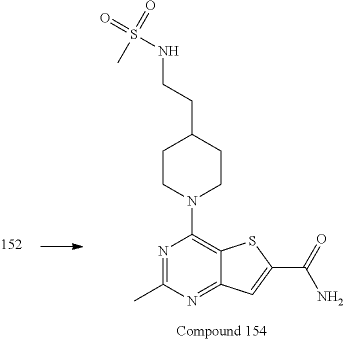 [SMILES: Cc1nc(N2CCC(CCNS(C)(=O)=O)CC2)c2sc(C(N)=O)cc2n1]

To a mixture of 4-(4-(2-aminoethyl)piperidin-1-yl)-2-methylthieno[3,2-d]pyrimidine-6-carboxamide (152; 0.090 g, 0.253 mmol) and triethylamine (0.5 mL) in pyridine (5 mL) was added methanesulfonyl chloride (0.032 g, 0.278 mmol) at 0° C. The mixture was stirred at room temperature overnight, quenched with 1 N ammonium hydroxide, and then concentrated in vacuo. The residue was neutralized to pH 7 using sat. Na₂CO₃ and the mixture extracted with CH₂Cl₂ (3 15 mL). The combined organic layers were washed with brine, dried and concentrated. The residue was purified by prep-TLC (1:10 MeOH in CH₂Cl₂) to obtain 2-methyl-4-(4-(2-(methylsulfonamido)ethyl)piperidin-1-yl)thieno[3,2-d]pyrimidine-6-carboxamide (Compound 154; 0.0126 g, 11%). MS (ESI) calcd for C₁₆H₂₃N₅O₃S₂: 397.12. found: 398 [M+H].

Compounds 155, 156, 157, 158 and 159 of Table 7 were prepared in an analogous manner.

### Example 39

### Preparation of 7-(4-(2-(cyclopentanecarboxamido)ethyl)piperidin-1-yl)thieno[2,3-c]pyridine-2-carboxamide (Compound 177)

### Step 1. Synthesis of 3-bromo-4-(dibromomethyl)pyridine (Compound 161)

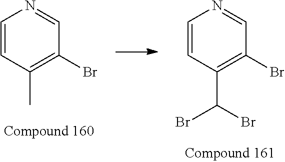

To a solution of 3-bromo-4-methylpyridine (Compound 160; 200 g, 1.16 mol) and AIBN (34.3 g, 209 mmol) in CCl₄ (1.5 L) was added NBS (414 g, 2.32 mol) in portions at room temperature. The reaction mixture was heated at reflux for 5 h. After cooling down to room temperature, the mixture was concentrated in vacuo, and the residue was purified by silica gel column chromatography to obtain 3-bromo-4-(dibromomethyl)pyridine (Compound 161; 58 g, 15%). MS (ESI) calcd=for C₆H4Br₃N═: 326.79.

### Step 2. Synthesis of 2-bromo-4-(diethoxymethyl)pyridine (Compound 162)

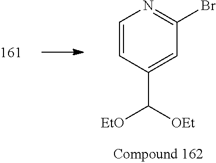 [SMILES: CCOC(OCC)c1ccnc(Br)c1]

To a solution of 3-bromo-4-(dibromomethyl)pyridine (161; 20.0 g, 60.8 mmol) in EtOH (200 mL) and water (200 mL) was added AgNO₃ (60.0 g, 313 mmol) at room temperature, the mixture was stirred at 70° C. overnight. After cooling down to room temperature, the resulting mixture was filtered, and the filtrate was then concentrated to obtain crude 2-bromo-4-(diethoxymethyl)pyridine (Compound 162; 15.0 g, 15%) which was used directly in the next step. MS (ESI) calcd for C₁₀H₁₄BrNO₂: 259.02.

### Step 3. Synthesis of 3-bromoisonicotinaldehyde (Compound 163)

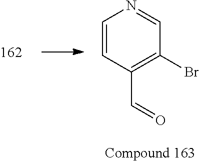

A mixture of crude 2-bromo-4-(diethoxymethyl)pyridine (162; 20.0 g, 76.9 mmol) and aqueous HBr (100 mL) was stirred at room temperature for 30 min, then neutralized with saturated NaHCO₃ (50 mL) to pH 8-10 at 0° C. The resulting mixture was extracted with CH₂Cl₂ (3×50 mL) and the combined organic layers were washed with brine, dried (MgSO₄) and concentrated. The residue was purified by silica gel column chromatography to obtain 3-bromoisonicotinaldehyde (Compound 163; 15.0 g, quantitative) as a white solid. MS (ESI) calcd for C₆H₄BrNO:184.95.

### Step 4. Synthesis of ethyl thieno[2,3-c]pyridine-2-carboxylate (Compound 164)

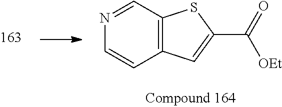 [SMILES: CCOC(=O)c1cc2ccncc2s1]

To a mixture of 3-bromoisonicotinaldehyde (163; 5.0 g, 27.2 mmol) and K₂CO₃ (5.3 g, 38.0 mmol) in DMF (100 ml) was added mercapto-acetic acid ethyl ester (3.3 g, 27.2 mmol) at room temperature. The reaction mixture was stirred overnight, then diluted with water (100 mL), extracted with CH₂Cl₂ (3×100 mL). The combined organic layers were washed with water (200 mL) and brine (200 mL), dried (Na₂SO₄), filtered and concentrated in vacuo. The residue was purified by silica gel column chromatography to obtain ethyl thieno[2,3-c]pyridine-2-carboxylate (Compound 164; 2.1 g, 35%). MS (ESI) calcd for C₁₀H₉NO₂S: 207.04.

### Step 5. Synthesis of 2-(ethoxycarbonyl)thieno[2,3-c]pyridine 6-oxide (Compound 165)

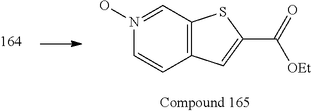

To a solution of ethyl thieno[2,3-c]pyridine-2-carboxylate (164; 1.4 g, 6.8 mmol) in CCl₄ (50 mL) was added meta-chloroperoxybenzoic acid (1.2 g, 20.3 mmol) in portions over 15 min at room temperature. The reaction mixture was heated at 70° C. and stirred overnight. After cooling down to room temperature, the mixture was diluted with saturated NaHCO₃ (50 mL), the organic layer was separated, and the aqueous phase was extracted with CH₂Cl₂ (3×100 mL). The combined organics were washed with water (200 mL), brine (200 mL), dried (Na₂SO₄), filtered and concentrated. The residue was purified by silica gel column chromatography to obtain 2-(ethoxycarbonyl)thieno[2,3-c]pyridine 6-oxide (Compound 165; 0.960 g, 64%) as a yellow solid. MS (ESI) calcd for C₁₀H₉NO₃S: 223.03.

### Step 6. Synthesis of ethyl 7-chlorothieno[2,3-c]pyridine-2-carboxylate (Compound 166)

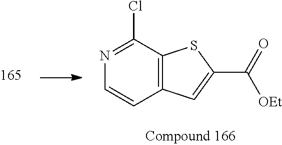 [SMILES: CCOC(=O)c1cc2ccnc(Cl)c2s1]

To a solution of 2-(ethoxycarbonyl)thieno[2,3-c]pyridine 6-oxide (165; 1.5 g, 6.7 mmol) in dioxane (30 mL) was added POCl₃ (0.7 g, 13.4 mmol). The mixture was stirred at 110° C. for 4 h. After cooling down to room temperature, the mixture was poured into water (50 mL), and neutralized with saturated NaHCO₃ to pH 8-10. The resulting mixture was then extracted with ethyl acetate (3×100 mL). The combined organic layers were washed with brine, dried (Na₂SO₄), and concentrated. The residue was purified by column chromatography to obtain ethyl 7-chlorothieno[2,3-c]pyridine-2-carboxylate (Compound 166; 0.900 g, 56%) as a white solid. MS (ESI) calcd for C₁₀H₈ClNO₂S: 241.00.

### Step 7. Synthesis of 7-chlorothieno[2,3-c]pyridine-2-carboxamide (Compound 167)

A mixture of ethyl 7-chlorothieno[2,3-c]pyridine-2-carboxylate (166; 0.950 g, 3.9 mmol) and 2N NH₃/MeOH (20 mL) was stirred at room temperature overnight. The solvent was removed, and the residue was purified by column chromatography to obtain 7-chlorothieno[2,3-c]pyridine-2-carboxamide as a white solid (Compound 167; 0.700 g, 97%). MS (ESI) calcd for C₁₀H₈ClNO₂S: 241.00.

### Step 8. Synthesis of tert-butyl (2-(1-(2-carbamoylthieno[2,3-c]pyridin-7-yl)piperidin-4-yl)ethyl)carbamate (Compound 168)

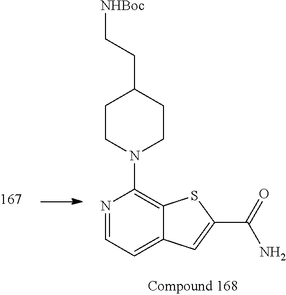 [SMILES: CC(C)(C)OC(=O)NCCC1CCN(c2nccc3cc(C(N)=O)sc23)CC1]

A mixture of 7-chlorothieno[2,3-c]pyridine-2-carboxamide (167; 0.100 g, 0.472 mmol), tert-butyl (2-(piperidin-4-yl)ethyl)carbamate (0.129 g, 0.566 mmol) and DIEA (0.122 g, 1.416 mmol) in NMP (2 mL) was microwave heated at 200° C. for 3 h. After cooling down to room temperature, the solvent was removed in vacuo and the residue was purified by Prep-TLC (1:30 MeOH in CH₂Cl₂) to obtain tert-butyl (2-(1-(2-carbamoylthieno[2,3-c]pyridin-7-yl)piperidin-4-yl)ethyl)carbamate (Compound 168; 0.039 g, 20%). MS (ESI) calcd for C₂₀H₂₈N₄O₃S: 404.19. found: 405 [M+H].

Compounds 169, 170, 171, 172, 173 and 174 of Table 7 were prepared in an analogous manner.

### Step 9. Synthesis of 7-(4-(2-aminoethyl)piperidin-1-yl)thieno[2,3-c]pyridine-2-carboxamide (Compound 175)

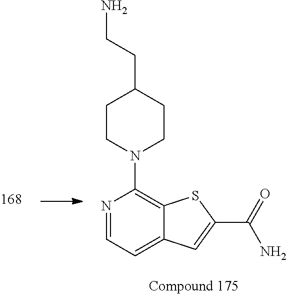 [SMILES: NCCC1CCN(c2nccc3cc(C(N)=O)sc23)CC1]

A mixture of tert-butyl (2-(1-(2-carbamoylthieno[2,3-c]pyridin-7-yl)piperidin-4-yl)ethyl)carbamate (168; 0.440 g, 1.13 mmol) in HCl/MeOH (1M, 10 mL) was stirred at room temperature overnight. The mixture was concentrated, the residue was dissolved in ammonium hydroxide, stirred for sever minutes, and concentrated again. The crude was purified by column chromatography (1:10 MeOH in CH₂Cl₂) to obtain 7-(4-(2-aminoethyl)piperidin-1-yl)thieno[2,3-c]pyridine-2-carboxamide (Compound 175; 0.217 g, 31%). MS (ESI) calcd for C₁₅H₂₀N₄OS: 304.14. found: 305 [M+H].

Compound 176 of Table 7 was prepared in an analogous manner.

### Step 10. Synthesis of 7-(4-(2-(cyclopentanecarboxamido)ethyl)piperidin-1-yl)thieno[2,3-c]pyridine-2-carboxamide (Compound 177)

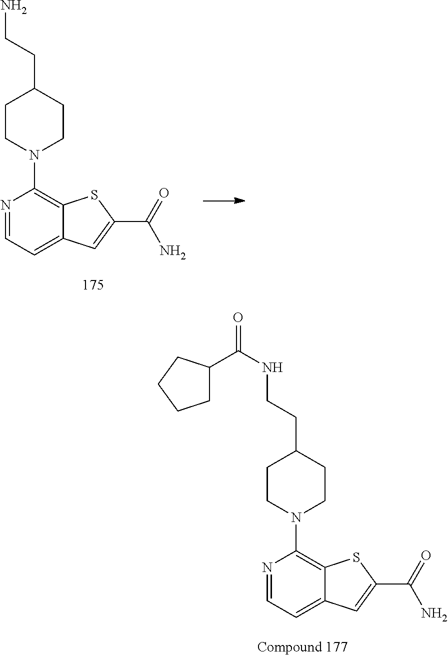 [SMILES: NCCC1CCN(c2nccc3cc(C(N)=O)sc23)CC1 ; NC(=O)c1cc2ccnc(N3CCC(CCNC(=O)C4CCCC4)CC3)c2s1]

To a mixture of 7-(4-(2-aminoethyl)piperidin-1-yl)thieno[2,3-c]pyridine-2-carboxamide (0.080 g, 0.262 mmol) and triethylamine (0.3 mL) in CH₂Cl₂ (6 mL) was added cyclopentanecarbonyl chloride (0.069 g, 0.525 mmol) dropwise at 0° C. over 15 min. The reaction mixture was warmed to room temperature and stirred overnight. The mixture was quenched with 1 N ammonium hydroxide and then concentrated in vacuo. The residue was purified by silica gel column chromatography (1:15 MeOH in CH₂Cl₂) to obtain 7-(4-(2-(cyclopentanecarboxamido)ethyl)piperidin-1-yl)thieno[2,3-c]pyridine-2-carboxamide (Compound 177; 0.023 g, 17%). MS (ESI) calcd for C₂₁H₂₈N₄O₂S: 400.19. found: 401 [M+H].

Compounds 178, 179, 180 and 181 of Table 7 were prepared in an analogous manner.

### Example 40

### Preparation of N-Methyl-4-(4-(2-pivalamidoethyl)piperidin-1-yl)thieno[3,2-d]pyrimidine-6-carboxamide (Compound 41)

### Step 1. Synthesis of 4-(4-(2-Pivalamidoethyl)piperidin-1-yl)thieno[3,2-d]pyrimidine-6-carboxylic acid 2,2,2-trifluoroacetate (Compound 40)

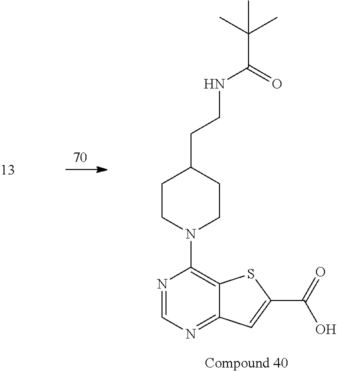 [SMILES: CC(C)(C)C(=O)NCCC1CCN(c2ncnc3cc(C(=O)O)sc23)CC1]

A solution of 4-chlorothieno[3,2-d]pyrimidine-6-carboxylic acid (13, 0.064 g, 0.3 mmol), N-(2-(piperidin-4-yl)ethyl)pivalamide (70, 0.064 g, 0.3 mmol) and DIEA (103 μl, 0.6 mmol) in CH₃CN (8 mL) was heated at 80° C. overnight. The reaction mixture was concentrated and purified by prep-HPLC and lyophilized to afford 4-(4-(2-Pivalamidoethyl)piperidin-1-yl)thieno[3,2-d]pyrimidine-6-carboxylic acid 2,2,2-trifluoroacetate (Compound 40; 0.080 g, 53%). MS (ESI) calcd for C₁₉H₂₆N₄O₃S: 390.17. found: 391 [M+H].

Compound 192 of Table 8 was prepared in an analogous manner

### Step 2. Synthesis of N-Methyl-4-(4-(2-pivalamidoethyl)piperidin-1-yl)thieno[3,2-d]pyrimidine-6-carboxamide (Compound 41)

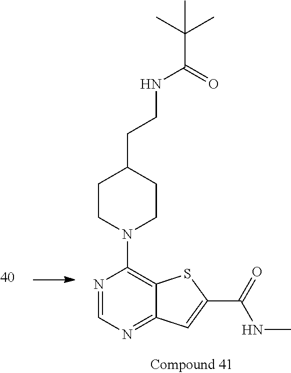 [SMILES: CNC(=O)c1cc2ncnc(N3CCC(CCNC(=O)C(C)(C)C)CC3)c2s1]

To a solution of 4-(4-(2-pivalamidoethyl)piperidin-1-yl)thieno[3,2-d]pyrimidine-6-carboxylic acid (40, 0.026 g, 0.051 mmol), HATU (0.058 g, 0.15 mmol), DIEA (44 μl, 0.25 mmol) in DMF (1 mL) was added methanamine hydrochloride (0.017 g, 0.25 mmol) and the reaction mixture was stirred for two days. The reaction mixture was diluted with aq. NaHCO₃ (sat.) and extracted with CH₂Cl₂ (2×). The organic layer was concentrated and purified by column chromatography (0 to 10% MeOH in CH₂Cl₂ gradient) to obtain N-Methyl-4-(4-(2-pivalamidoethyl)piperidin-1-yl)thieno[3,2-d]pyrimidine-6-carboxamide (Compound 41; 0.007 g, 34%). MS (ESI) calcd for C₂₀H₂₉N₅O₂S: 403.20. found: 404 [M+H].

### Example 41

### Preparation of 4-(Piperidin-1-yl)thieno[3,2-d]pyrimidine-6-carboxamide. (Compound 21)

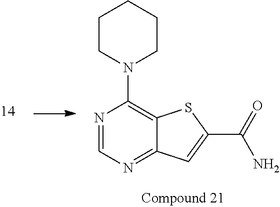 [SMILES: NC(=O)c1cc2ncnc(N3CCCCC3)c2s1]

A solution of 4-chlorothieno[3,2-d]pyrimidine-6-carboxamide (14, 0.085 g, 0.500 mmol) and piperidine (0.34 g, 4 mmol) in CH₃CN (5 mL) was heated at 60° C. for 18 hours. The reaction mixture was concentrated to dryness and purified by prep-HPLC to obtain 4-(piperidin-1-yl)thieno[3,2-d]pyrimidine-6-carboxamide (Compound 21; 0.086 g, 82%). MS (ESI) calcd for C₁₂H₁₄N₄OS: 262.09. found: 263 [M+H].

### Example 42

### Preparation of 4-(Ethylamino)thieno[3,2-d]pyrimidine-6-carboxamide. (Compound 22)

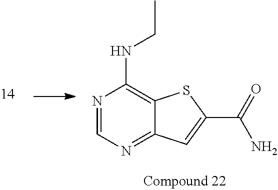

A solution of 4-chlorothieno[3,2-d]pyrimidine-6-carboxamide (14, 0.085 g, 0.500 mmol) and 70% ethylamine in water (200 μL, excess) in CH₃CN (5 mL) was heated at 60° C. for 18 hours. The reaction mixture was concentrated to dryness and purified by prep-HPLC to obtain 4-(ethylamino)thieno[3,2-d]pyrimidine-6-carboxamide (Compound 22; 0.083 g, 93%). MS (ESI) calcd for C₉H₁₀N₄OS: 222.06. found: 223 [M+H].

### Example 43

### Preparation of N-(2-(1-(thieno[3,2-d]pyrimidin-4-yl)piperidin-4-yl)ethyl)pivalamide (Compound 42)

### Step 1. Synthesis of tert-butyl (2-(1-(thieno[3,2-d]pyrimidin-4-yl)piperidin-4-yl)ethyl)carbamate (Compound 187)

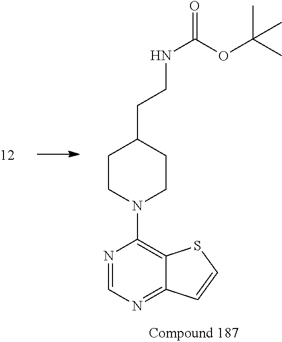 [SMILES: CC(C)(C)OC(=O)NCCC1CCN(c2ncnc3ccsc23)CC1]

To a solution of 4-chlorothieno[3,2-d]pyrimidine (12, 0.205 g, 1.20 mmol) and diisopropylethylamine (417 μL, 2.4 mmol) in acetonitrile was added tert-butyl (2-(piperidin-4-yl)ethyl)carbamate (0.357 g, 1.56 mmol). The resulting solution was stirred at 50° C. for 2 h. The reaction mixture was concentrated under reduced pressure, dissolved in ethyl acetate and washed with brine. The organic layer was dried (Na₂SO₄), filtered and concentrated under reduced pressure to afford tert-butyl (2-(1-(thieno[3,2-d]pyrimidin-4-yl)piperidin-4-yl)ethyl)carbamate (Compound 187; 0.600 g, assumed quantitative) as an oil. The product was used without further purification. MS (ESI) calcd for C₁₈H₂₆N₄O₂S: 362.18.

### Step 2. Synthesis of 2-(1-(thieno[3,2-d]pyrimidin-4-yl)piperidin-4-yl)ethanamine 2,2,2-trifluoroacetate (Compound 188)

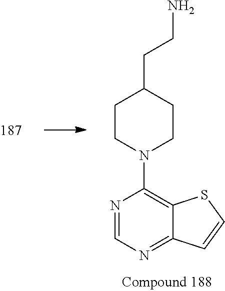 [SMILES: NCCC1CCN(c2ncnc3ccsc23)CC1]

A solution of tert-butyl (2-(1-(thieno[3,2-d]pyrimidin-4-yl)piperidin-4-yl)ethyl)carbamate (187, assumed 1.20 mmol) from the above reaction was dissolved in dichloromethane and treated at room temperature with of trifluoroacetic acid (924 μL, 12 mmol). The resulting solution was stirred overnight at room temperature and then concentrated under reduced pressure to afford 2-(1-(thieno[3,2-d]pyrimidin-4-yl)piperidin-4-yl)ethanamine mono triflate salt (Compound 188; assumed quantitative, 1.20 mmol), which was used without further purification. MS (ESI) calcd for C₁₃H₁₈N₄S: 262.13. found: 263 [M+H].

### Step 3. Synthesis of N-(2-(1-(thieno[3,2-d]pyrimidin-4-yl)piperidin-4-yl)ethyl)pivalamide (Compound 42)

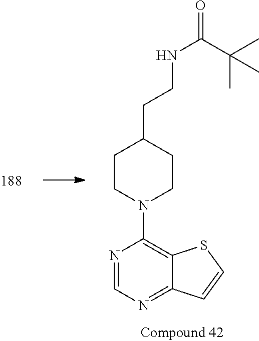 [SMILES: CC(C)(C)C(=O)NCCC1CCN(c2ncnc3ccsc23)CC1]

2-(1-(Thieno[3,2-d]pyrimidin-4-yl)piperidin-4-yl)ethanamine mono triflate salt (188, assumed 1.20 mmol), from the above reaction was dissolved in a biphasic mixture of ethyl acetate (20 mL) and 1M Na₂CO₃ (20 mL). While stirring rapidly, the mixture was treated with pivaloyl chloride (295 μL, 2.4 mmol) at room temperature and allowed to stir for an additional 16 h. The organic layer was separated and washed once with NaHCO₃ (20 mL). The organic layer was dried (Na₂SO₄) and concentrated under reduced pressure. The crude product was purified with a silica gel cartridge (24 g) using a gradient eluent of 0 to 8% MeOH in CH₂Cl₂. Evaporation of solvent yielded a colorless oil which was triturated with ether/ethyl acetate to afford N-(2-(1-(thieno[3,2-d]pyrimidin-4-yl)piperidin-4-yl)ethyl)pivalamide (Compound 42) as a white solid. MS (ESI) calcd for C₁₈H₂₆N₄OS: 346.18. found: 347 [M+H].

### Example 44

### Preparation of 4-(4-(2-((tert-butoxycarbonyl)amino)ethyl)piperidin-1-yl)thieno[3,2-d]pyrimidine-6-carboxylic acid. (Compound 189)

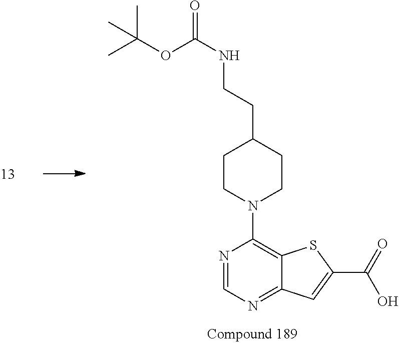 [SMILES: CC(C)(C)OC(=O)NCCC1CCN(c2ncnc3cc(C(=O)O)sc23)CC1]

To a solution of 4-chlorothieno[3,2-d]pyrimidine-6-carboxylic acid (13, 0.213 g, 0.992 mmol) and in CH₃CN was added triethylamine (0.415 mL, 2.98 mmol) followed by tert-butyl (2-(piperidin-4-yl)ethyl)carbamate (0.272 g, 1.2 mmol). The reaction mixture was stirred at 60° C. for 1.5 hr and then concentrated under reduced pressure. The reaction mixture was adjusted to pH 4-5 and extracted with ethyl acetate. The insoluble solids were collected by filtration, dissolved in a mixture of CH₂Cl₂/MeOH, dried (Na₂SO₄), filtered and concentrated to afford 4-(4-(2-((tert-butoxycarbonyl)amino)ethyl)piperidin-1-yl)thieno[3,2-d]pyrimidine-6-carboxylic acid (100 mg, 25%). MS (ESI) calcd for C₁₉H₂₆N₄O₄S: 406.17. found: 407 [M+H].

Sirtuin-modulating compounds of Formula (I) that inhibited SIRT1, SIRT2 and SIRT3 were identified using the assay described above and are shown below in Table 7. The IC₅₀ values refer to the dose of a drug which produces 50% of its maximum response or effect. In other words, it is the half maximal inhibitory concentration of a drug. The IC₅₀ values for the inhibiting compounds of Formula (I) are represented by A (EC_{1.5}<1 μM), B (EC_{1.5} 1-10 μM), C (EC_{1.5}>10 μM). “NT” means not tested; “ND” means not determinable.

TABLE 7
Compounds of Formula (I).

| Compound | [M + H]+ | | Trp | | |
|---|---|---|---|---|---|
| No. | (Calc) | Structure | SIRT1_IC₅₀ | SIRT2_IC₅₀ | SIRT3_IC₅₀ |
| 11a | 482 | 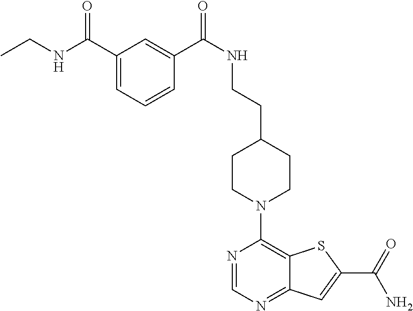 [SMILES: CCNC(=O)c1cccc(C(=O)NCCC2CCN(c3ncnc4cc(C(N)=O)sc34)CC2)c1] | A | A | A |
| 11b | 483 | 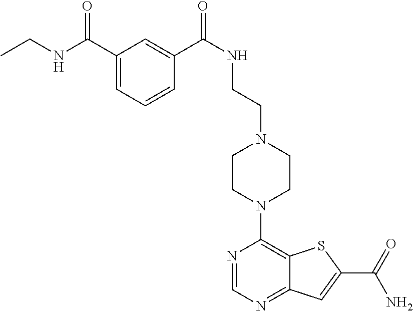 [SMILES: CCNC(=O)c1cccc(C(=O)NCCN2CCN(c3ncnc4cc(C(N)=O)sc34)CC2)c1] | A | A | A |
| 11c | 488 | 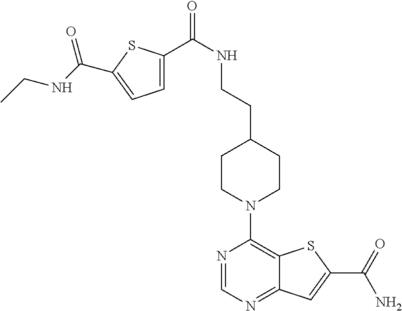 [SMILES: CCNC(=O)c1ccc(C(=O)NCCC2CCN(c3ncnc4cc(C(N)=O)sc34)CC2)s1] | A | A | A |
| 11d | 489 | 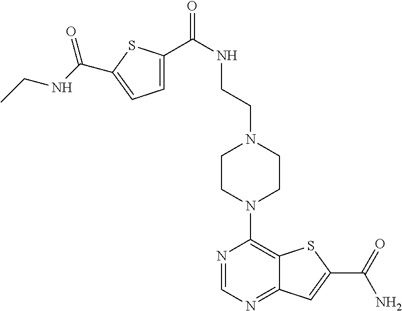 [SMILES: CCNC(=O)c1ccc(C(=O)NCCN2CCN(c3ncnc4cc(C(N)=O)sc34)CC2)s1] | A | A | A |
| 15a | 407 | 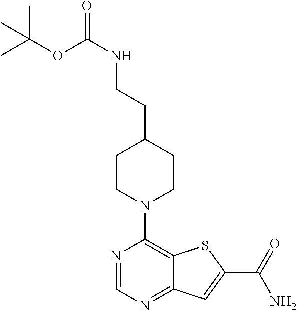 [SMILES: CC(C)(C)OC(=O)NCCC1CCN(c2ncnc3cc(C(N)=O)sc23)CC1] | A | A | A |
| 15b | 408 | 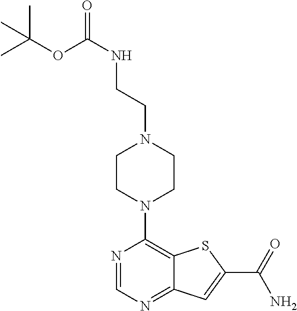 [SMILES: CC(C)(C)OC(=O)NCCN1CCN(c2ncnc3cc(C(N)=O)sc23)CC1] | A | A | A |
| 16a | 307 | 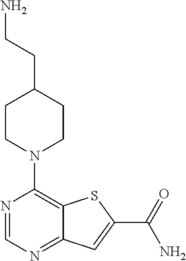 [SMILES: NCCC1CCN(c2ncnc3cc(C(N)=O)sc23)CC1] | B | A | A |
| 16b | 308 | 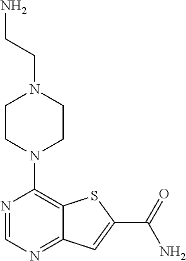 [SMILES: NCCN1CCN(c2ncnc3cc(C(N)=O)sc23)CC1] | B | A | A |
| 17 | 517 | 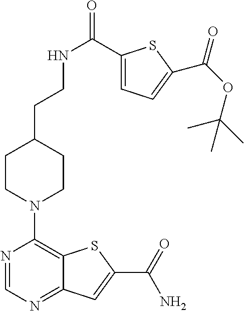 [SMILES: CC(C)(C)OC(=O)c1ccc(C(=O)NCCC2CCN(c3ncnc4cc(C(N)=O)sc34)CC2)s1] | A | A | A |
| 18 | 461 | 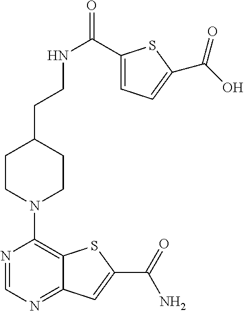 [SMILES: NC(=O)c1cc2ncnc(N3CCC(CCNC(=O)c4ccc(C(=O)O)s4)CC3)c2s1] | A | A | A |
| 19 | 417 | 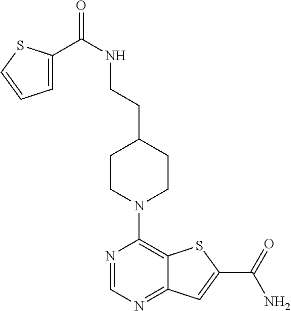 [SMILES: NC(=O)c1cc2ncnc(N3CCC(CCNC(=O)c4cccs4)CC3)c2s1] | A | A | A |
| 20 | 349 | 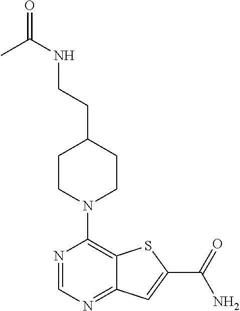 [SMILES: CC(=O)NCCC1CCN(c2ncnc3cc(C(N)=O)sc23)CC1] | A | A | A |
| 23 | 335 | 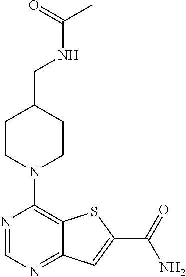 [SMILES: CC(=O)NCC1CCN(c2ncnc3cc(C(N)=O)sc23)CC1] | C | B | B |
| 24 | 350 | 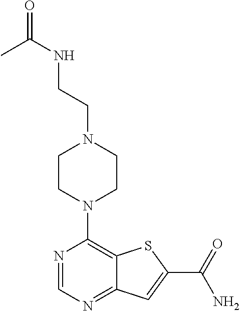 [SMILES: CC(=O)NCCN1CCN(c2ncnc3cc(C(N)=O)sc23)CC1] | B | A | A |
| 25 | 365 | 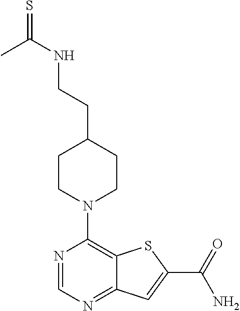 [SMILES: CC(=S)NCCC1CCN(c2ncnc3cc(C(N)=O)sc23)CC1] | A | A | A |
| 26 | 366 | 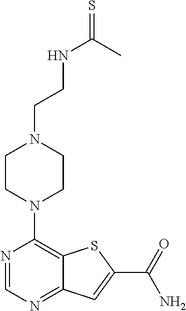 [SMILES: CC(=S)NCCN1CCN(c2ncnc3cc(C(N)=O)sc23)CC1] | A | A | A |
| 27 | 377 | 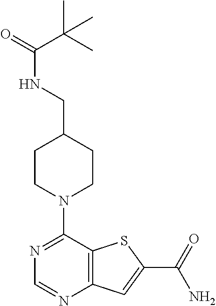 [SMILES: CC(C)(C)C(=O)NCC1CCN(c2ncnc3cc(C(N)=O)sc23)CC1] | B | A | A |
| 28 | 391 | 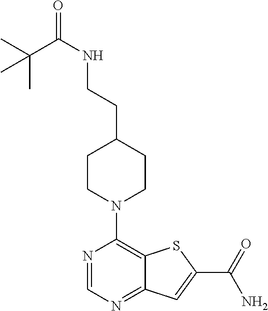 [SMILES: CC(C)(C)C(=O)NCCC1CCN(c2ncnc3cc(C(N)=O)sc23)CC1] | A | A | A |
| 29 | 405 | 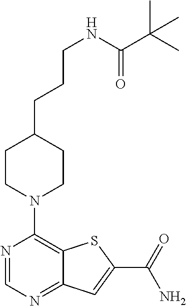 [SMILES: CC(C)(C)C(=O)NCCCC1CCN(c2ncnc3cc(C(N)=O)sc23)CC1] | A | A | A |
| 30 | 392 | 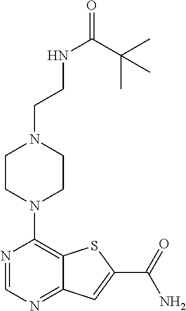 [SMILES: CC(C)(C)C(=O)NCCN1CCN(c2ncnc3cc(C(N)=O)sc23)CC1] | A | A | A |
| 31 | 385 | 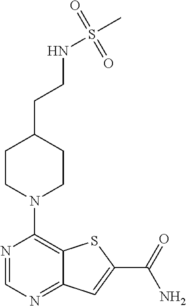 [SMILES: CS(=O)(=O)NCCC1CCN(c2ncnc3cc(C(N)=O)sc23)CC1] | A | A | A |
| 32 | 386 | 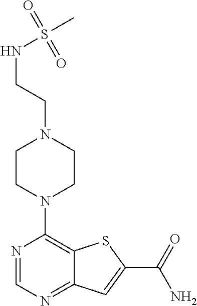 [SMILES: CS(=O)(=O)NCCN1CCN(c2ncnc3cc(C(N)=O)sc23)CC1] | A | A | A |
| 33 | 347 | 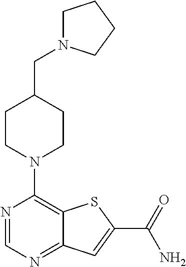 [SMILES: NC(=O)c1cc2ncnc(N3CCC(CN4CCCC4)CC3)c2s1] | A | A | A |
| 34 | 361 | 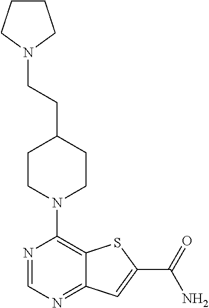 [SMILES: NC(=O)c1cc2ncnc(N3CCC(CCN4CCCC4)CC3)c2s1] | A | A | A |
| 35 | 362 | 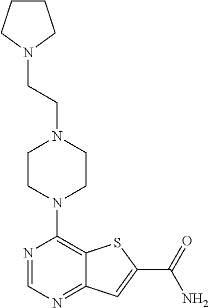 [SMILES: NC(=O)c1cc2ncnc(N3CCN(CCN4CCCC4)CC3)c2s1] | A | A | A |
| 36 | 375 | 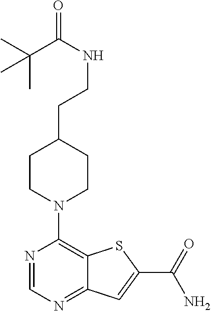 [SMILES: CC(C)(C)C(=O)NCCC1CCN(c2ncnc3cc(C(N)=O)sc23)CC1] | A | A | B |
| 37 | 390 | 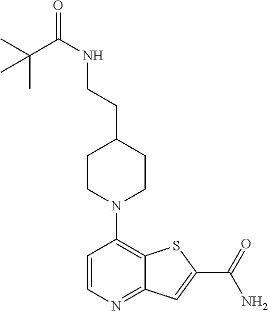 [SMILES: CC(C)(C)C(=O)NCCC1CCN(c2ccnc3cc(C(N)=O)sc23)CC1] | A | A | A |
| 38 | 390 | 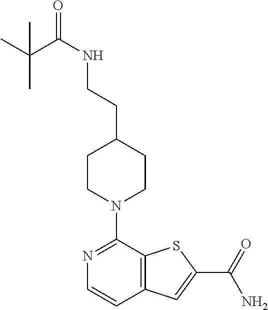 [SMILES: CC(C)(C)C(=O)NCCC1CCN(c2nccc3cc(C(N)=O)sc23)CC1] | A | A | B |
| 39 | 405 | 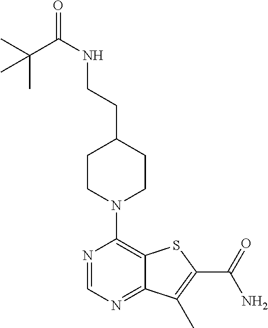 [SMILES: Cc1c(C(N)=O)sc2c(N3CCC(CCNC(=O)C(C)(C)C)CC3)ncnc12] | A | A | A |
| 43 | 321 | 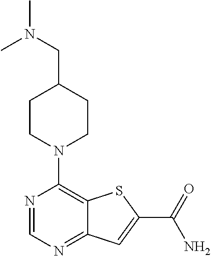 [SMILES: CN(C)CC1CCN(c2ncnc3cc(C(N)=O)sc23)CC1] | B | A | A |
| 44 | 309 | 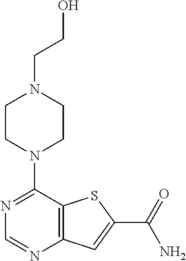 [SMILES: NC(=O)c1cc2ncnc(N3CCN(CCO)CC3)c2s1] | B | A | B |
| 45 | 323 | 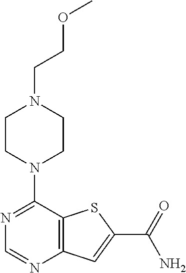 [SMILES: COCCN1CCN(c2ncnc3cc(C(N)=O)sc23)CC1] | B | A | B |
| 46 | 335 | 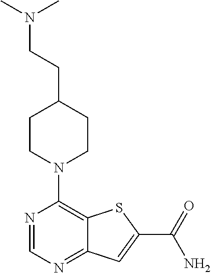 [SMILES: CN(C)CCC1CCN(c2ncnc3cc(C(N)=O)sc23)CC1] | B | A | A |
| 47 | 375 | 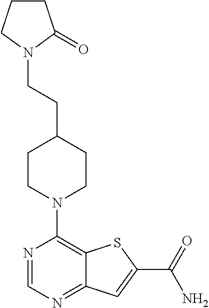 [SMILES: NC(=O)c1cc2ncnc(N3CCC(CCN4CCCC4=O)CC3)c2s1] | A | A | A |
| 48 | 335 | 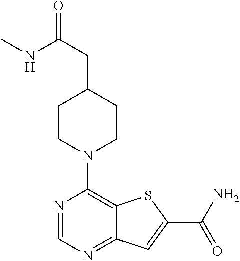 [SMILES: CNC(=O)CC1CCN(c2ncnc3cc(C(N)=O)sc23)CC1] | B | B | B |
| 49 | 322 | 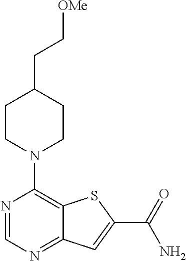 [SMILES: COCCC1CCN(c2ncnc3cc(C(N)=O)sc23)CC1] | A | A | A |
| 50 | 308 | 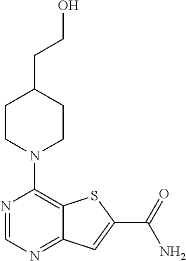 [SMILES: NC(=O)c1cc2ncnc(N3CCC(CCO)CC3)c2s1] | A | A | A |
| 51 | 294 | 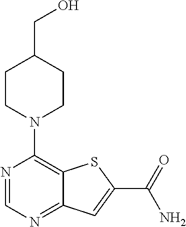 [SMILES: NC(=O)c1cc2ncnc(N3CCC(CO)CC3)c2s1] | B | A | A |
| 52 | 358 | 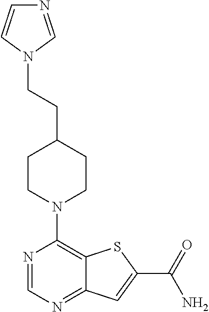 [SMILES: NC(=O)c1cc2ncnc(N3CCC(CCn4ccnc4)CC3)c2s1] | A | A | A |
| 53 | 403 | 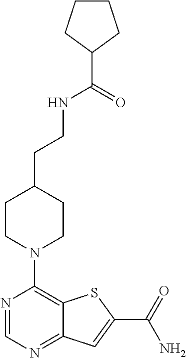 [SMILES: NC(=O)c1cc2ncnc(N3CCC(CCNC(=O)C4CCCC4)CC3)c2s1] | A | A | A |
| 55 | 307 | 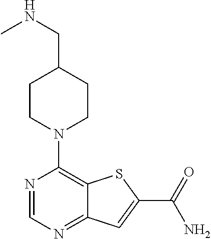 [SMILES: CNCC1CCN(c2ncnc3cc(C(N)=O)sc23)CC1] | C | B | B |
| 56 | 347 | 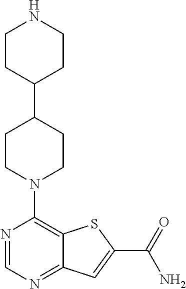 [SMILES: NC(=O)c1cc2ncnc(N3CCC(C4CCNCC4)CC3)c2s1] | A | A | A |
| 57 | 393 | 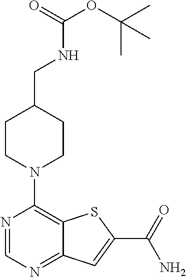 [SMILES: CC(C)(C)OC(=O)NCC1CCN(c2ncnc3cc(C(N)=O)sc23)CC1] | NT | B | C |
| 58 | 363 | 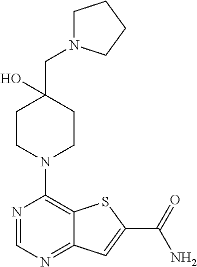 [SMILES: NC(=O)c1cc2ncnc(N3CCC(O)(CN4CCCC4)CC3)c2s1] | B | B | B |
| 59 | 293 | 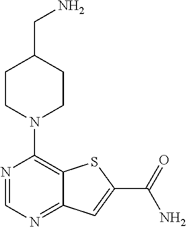 [SMILES: NCC1CCN(c2ncnc3cc(C(N)=O)sc23)CC1] | C | C | C |
| 63 | 404 | 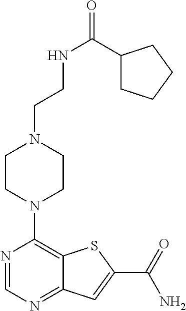 [SMILES: NC(=O)c1cc2ncnc(N3CCN(CCNC(=O)C4CCCC4)CC3)c2s1] | A | A | A |
| 67 | 475 | 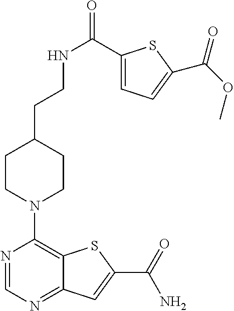 [SMILES: COC(=O)c1ccc(C(=O)NCCC2CCN(c3ncnc4cc(C(N)=O)sc34)CC2)s1] | A | A | A |
| 76 | 421 | 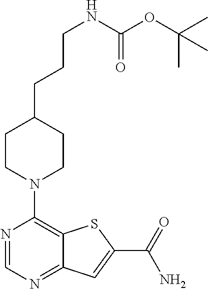 [SMILES: CC(C)(C)OC(=O)NCCCC1CCN(c2ncnc3cc(C(N)=O)sc23)CC1] | A | A | A |
| 77 | 321 | 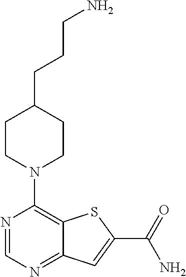 [SMILES: NCCCC1CCN(c2ncnc3cc(C(N)=O)sc23)CC1] | A | A | A |
| 78 | 378 | 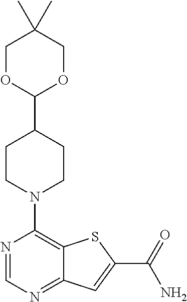 [SMILES: CC1(C)COC(C2CCN(c3ncnc4cc(C(N)=O)sc34)CC2)OC1] | B | B | B |
| 85 | 391 | 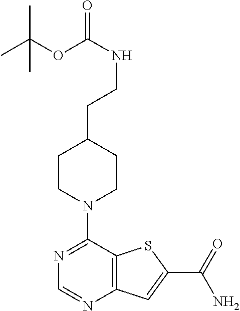 [SMILES: CC(C)(C)OC(=O)NCCC1CCN(c2ncnc3cc(C(N)=O)sc23)CC1] | A | A | A |
| 86 | 392 | 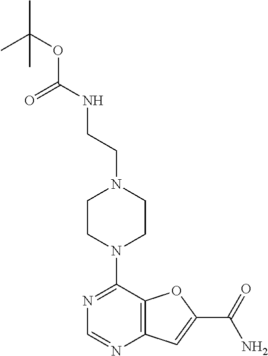 [SMILES: CC(C)(C)OC(=O)NCCN1CCN(c2ncnc3cc(C(N)=O)oc23)CC1] | A | A | B |
| 87 | 345 | 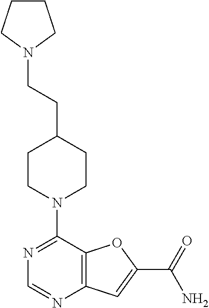 [SMILES: NC(=O)c1cc2ncnc(N3CCC(CCN4CCCC4)CC3)c2o1] | B | A | A |
| 88 | 387 | 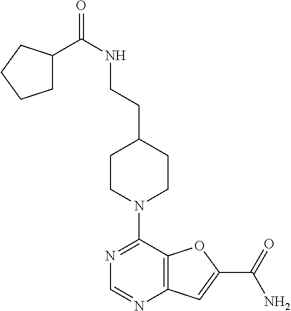 [SMILES: NC(=O)c1cc2ncnc(N3CCC(CCNC(=O)C4CCCC4)CC3)c2o1] | A | A | A |
| 89 | 370 | 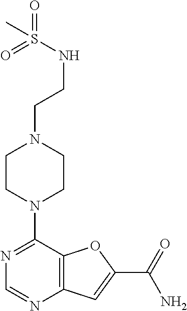 [SMILES: CS(=O)(=O)NCCN1CCN(c2ncnc3cc(C(N)=O)oc23)CC1] | B | A | A |
| 90 | 331 | 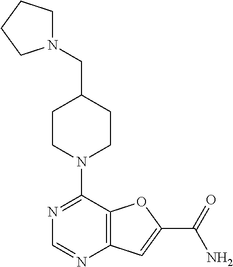 [SMILES: NC(=O)c1cc2ncnc(N3CCC(CN4CCCC4)CC3)c2o1] | B | B | A |
| 91 | 306 | 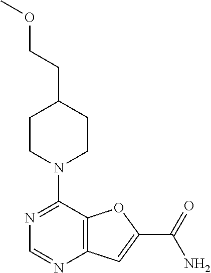 [SMILES: COCCC1CCN(c2ncnc3cc(C(N)=O)oc23)CC1] | B | B | B |
| 92 | 346 | 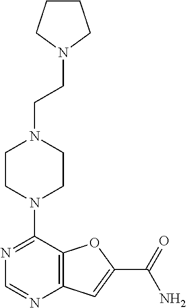 [SMILES: NC(=O)c1cc2ncnc(N3CCN(CCN4CCCC4)CC3)c2o1] | B | A | A |
| 93 | 292 | 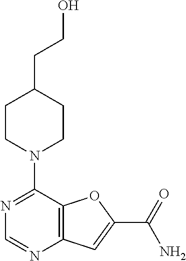 [SMILES: NC(=O)c1cc2ncnc(N3CCC(CCO)CC3)c2o1] | B | A | A |
| 94 | 431 | 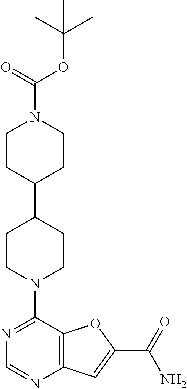 [SMILES: CC(C)(C)OC(=O)N1CCC(C2CCN(c3ncnc4cc(C(N)=O)oc34)CC2)CC1] | B | B | C |
| 95 | 291 | 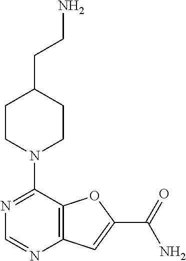 [SMILES: NCCC1CCN(c2ncnc3cc(C(N)=O)oc23)CC1] | B | A | B |
| 96 | 292 | 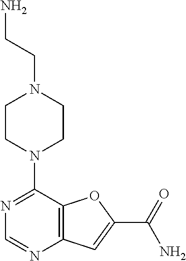 [SMILES: NCCN1CCN(c2ncnc3cc(C(N)=O)oc23)CC1] | C | B | B |
| 97 | 376 | 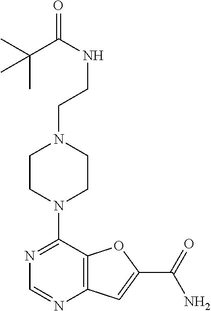 [SMILES: CC(C)(C)C(=O)NCCN1CCN(c2ncnc3cc(C(N)=O)oc23)CC1] | A | A | A |
| 98 | 388 | 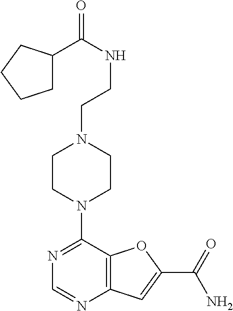 [SMILES: NC(=O)c1cc2ncnc(N3CCN(CCNC(=O)C4CCCC4)CC3)c2o1] | A | A | A |
| 99 | 369 | 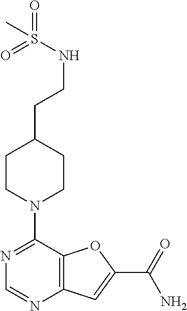 [SMILES: CS(=O)(=O)NCCC1CCN(c2ncnc3cc(C(N)=O)oc23)CC1] | A | A | A |
| 100 | 331 | 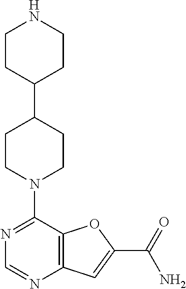 [SMILES: NC(=O)c1cc2ncnc(N3CCC(C4CCNCC4)CC3)c2o1] | C | C | B |
| 103 | 406 | 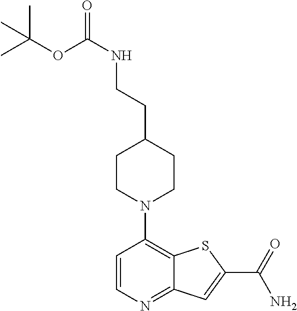 [SMILES: CC(C)(C)OC(=O)NCCC1CCN(c2ccnc3cc(C(N)=O)sc23)CC1] | A | A | A |
| 104 | 306 | 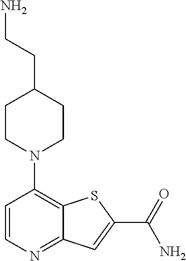 [SMILES: NCCC1CCN(c2ccnc3cc(C(N)=O)sc23)CC1] | B | A | A |
| 105 | 346 | 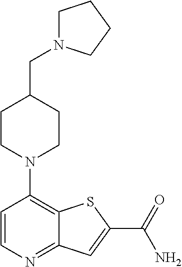 [SMILES: NC(=O)c1cc2nccc(N3CCC(CN4CCCC4)CC3)c2s1] | B | A | A |
| 112 | 468 | 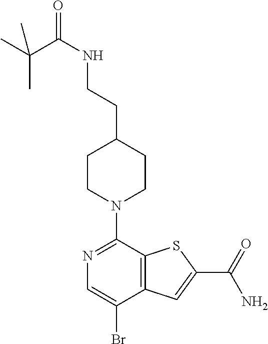 [SMILES: CC(C)(C)C(=O)NCCC1CCN(c2ncc(Br)c3cc(C(N)=O)sc23)CC1] | B | B | C |
| 116 | 421 | 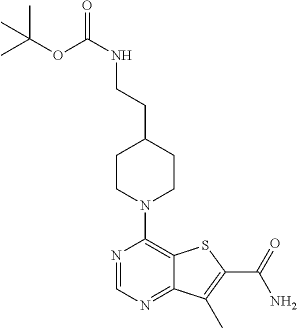 [SMILES: Cc1c(C(N)=O)sc2c(N3CCC(CCNC(=O)OC(C)(C)C)CC3)ncnc12] | A | A | A |
| 117 | 321 | 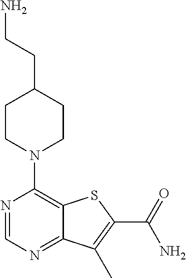 [SMILES: Cc1c(C(N)=O)sc2c(N3CCC(CCN)CC3)ncnc12] | C | B | B |
| 122 | 508 | 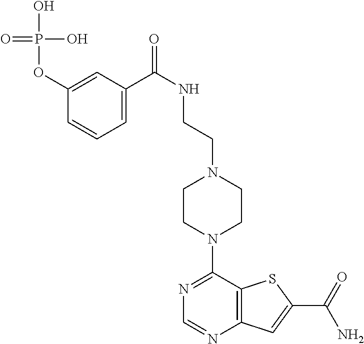 [SMILES: NC(=O)c1cc2ncnc(N3CCN(CCNC(=O)c4cccc(OP(=O)(O)O)c4)CC3)c2s1] | A | A | A |
| 127 | 498 | 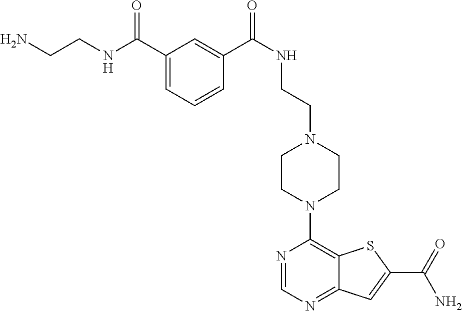 [SMILES: NCCNC(=O)c1cccc(C(=O)NCCN2CCN(c3ncnc4cc(C(N)=O)sc34)CC2)c1] | A | A | A |
| 131 | 429 | 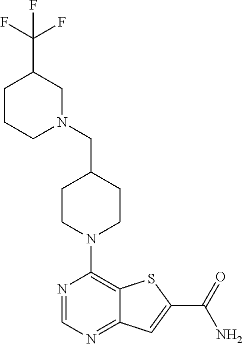 [SMILES: NC(=O)c1cc2ncnc(N3CCC(CN4CCCC(C(F)(F)F)C4)CC3)c2s1] | A | A | A |
| 132 | 415 | 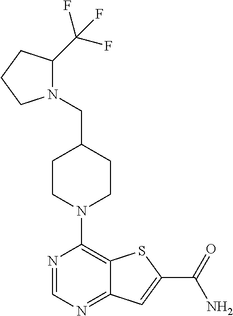 [SMILES: NC(=O)c1cc2ncnc(N3CCC(CN4CCCC4C(F)(F)F)CC3)c2s1] | A | A | A |
| 136 | 560 | 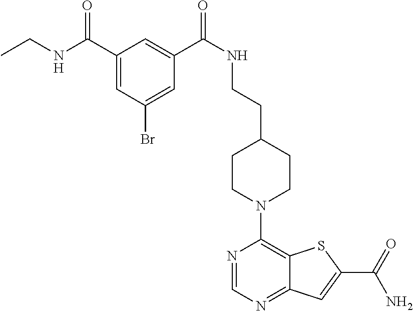 [SMILES: CCNC(=O)c1cc(Br)cc(C(=O)NCCC2CCN(c3ncnc4cc(C(N)=O)sc34)CC2)c1] | A | A | A |
| 145 | 421 | 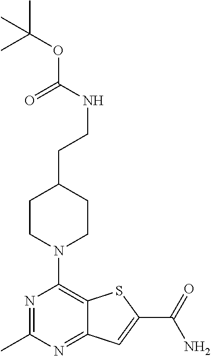 [SMILES: Cc1nc(N2CCC(CCNC(=O)OC(C)(C)C)CC2)c2sc(C(N)=O)cc2n1] | B | A | A |
| 146 | 375 | 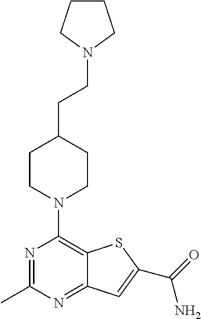 [SMILES: Cc1nc(N2CCC(CCN3CCCC3)CC2)c2sc(C(N)=O)cc2n1] | B | B | A |
| 147 | 361 | 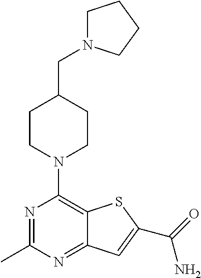 [SMILES: Cc1nc(N2CCC(CN3CCCC3)CC2)c2sc(C(N)=O)cc2n1] | C | B | B |
| 148 | 336 | 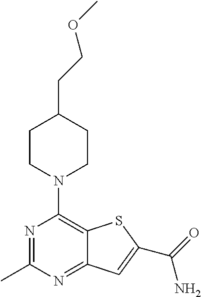 [SMILES: COCCC1CCN(c2nc(C)nc3cc(C(N)=O)sc23)CC1] | B | B | B |
| 149 | 422 | 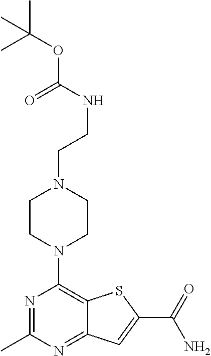 [SMILES: Cc1nc(N2CCN(CCNC(=O)OC(C)(C)C)CC2)c2sc(C(N)=O)cc2n1] | C | B | B |
| 150 | 376 | 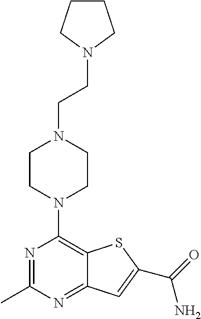 [SMILES: Cc1nc(N2CCN(CCN3CCCC3)CC2)c2sc(C(N)=O)cc2n1] | B | B | A |
| 151 | 322 | 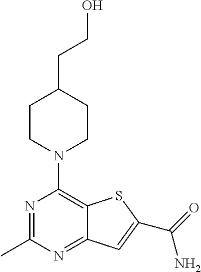 [SMILES: Cc1nc(N2CCC(CCO)CC2)c2sc(C(N)=O)cc2n1] | B | B | B |
| 152 | 321 | 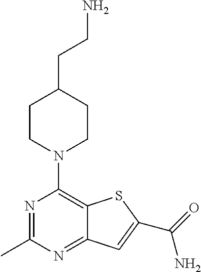 [SMILES: Cc1nc(N2CCC(CCN)CC2)c2sc(C(N)=O)cc2n1] | C | B | B |
| 153 | 322 | 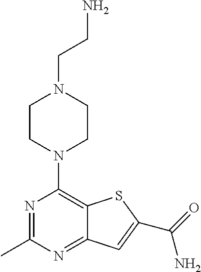 [SMILES: Cc1nc(N2CCN(CCN)CC2)c2sc(C(N)=O)cc2n1] | C | B | B |
| 154 | 399 | 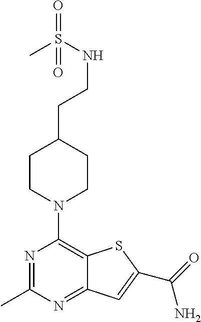 [SMILES: Cc1nc(N2CCC(CCNS(C)(=O)=O)CC2)c2sc(C(N)=O)cc2n1] | NT | NT | NT |
| 155 | 417 | 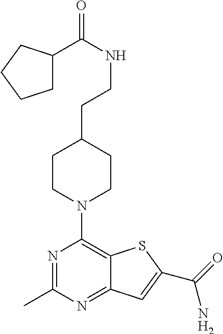 [SMILES: Cc1nc(N2CCC(CCNC(=O)C3CCCC3)CC2)c2sc(C(N)=O)cc2n1] | NT | NT | NT |
| 156 | 406 | 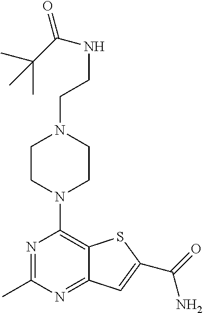 [SMILES: Cc1nc(N2CCN(CCNC(=O)C(C)(C)C)CC2)c2sc(C(N)=O)cc2n1] | B | A | B |
| 157 | 418 | 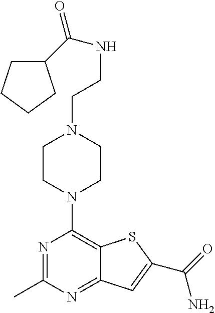 [SMILES: Cc1nc(N2CCN(CCNC(=O)C3CCCC3)CC2)c2sc(C(N)=O)cc2n1] | B | A | B |
| 158 | 400 | 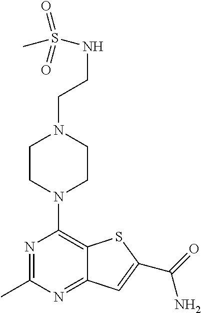 [SMILES: Cc1nc(N2CCN(CCNS(C)(=O)=O)CC2)c2sc(C(N)=O)cc2n1] | C | B | B |
| 159 | 405 | 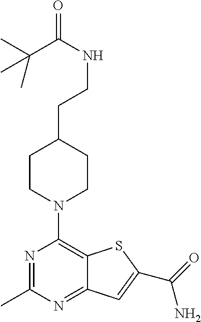 [SMILES: Cc1nc(N2CCC(CCNC(=O)C(C)(C)C)CC2)c2sc(C(N)=O)cc2n1] | A | A | B |
| 168 | 406 | 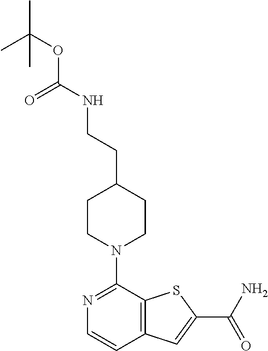 [SMILES: CC(C)(C)OC(=O)NCCC1CCN(c2nccc3cc(C(N)=O)sc23)CC1] | B | B | B |
| 169 | 360 | 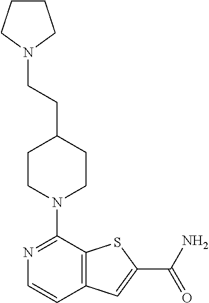 [SMILES: NC(=O)c1cc2ccnc(N3CCC(CCN4CCCC4)CC3)c2s1] | B | B | A |
| 170 | 346 | 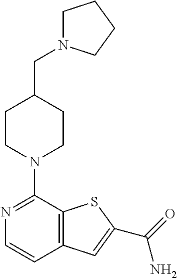 [SMILES: NC(=O)c1cc2ccnc(N3CCC(CN4CCCC4)CC3)c2s1] | C | C | B |
| 171 | 321 | 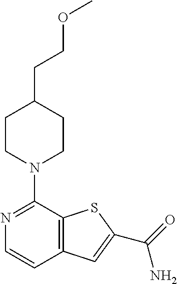 [SMILES: COCCC1CCN(c2nccc3cc(C(N)=O)sc23)CC1] | C | C | C |
| 172 | 407 | 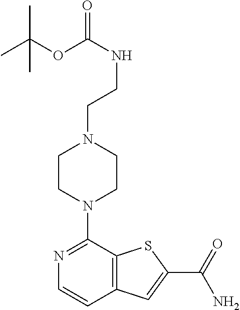 [SMILES: CC(C)(C)OC(=O)NCCN1CCN(c2nccc3cc(C(N)=O)sc23)CC1] | NT | NT | NT |
| 173 | 361 | 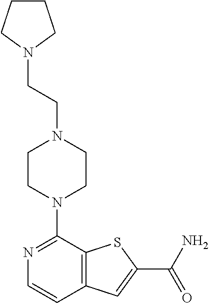 [SMILES: NC(=O)c1cc2ccnc(N3CCN(CCN4CCCC4)CC3)c2s1] | B | B | A |
| 174 | 307 | 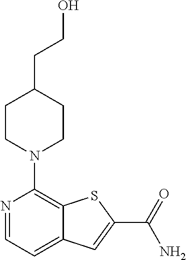 [SMILES: NC(=O)c1cc2ccnc(N3CCC(CCO)CC3)c2s1] | NT | NT | NT |
| 175 | 511 | 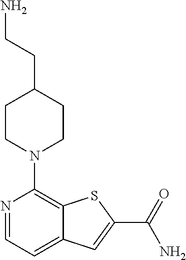 [SMILES: NCCC1CCN(c2nccc3cc(C(N)=O)sc23)CC1] | C | C | C |
| 176 | 307 | 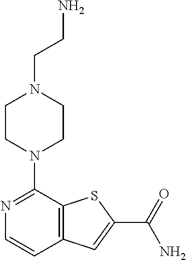 [SMILES: NCCN1CCN(c2nccc3cc(C(N)=O)sc23)CC1] | NT | NT | NT |
| 177 | 402 | 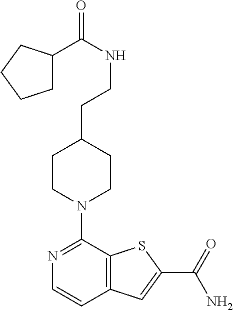 [SMILES: NC(=O)c1cc2ccnc(N3CCC(CCNC(=O)C4CCCC4)CC3)c2s1] | NT | NT | NT |
| 178 | 384 | 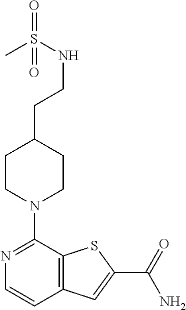 [SMILES: CS(=O)(=O)NCCC1CCN(c2nccc3cc(C(N)=O)sc23)CC1] | NT | NT | NT |
| 179 | 391 | 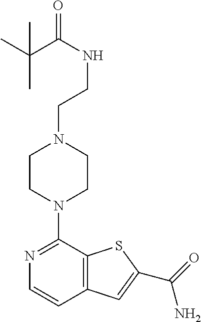 [SMILES: CC(C)(C)C(=O)NCCN1CCN(c2nccc3cc(C(N)=O)sc23)CC1] | C | B | C |
| 180 | 403 | 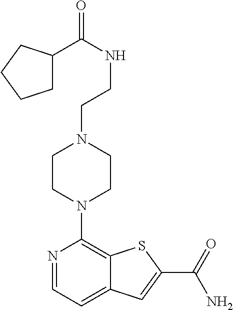 [SMILES: NC(=O)c1cc2ccnc(N3CCN(CCNC(=O)C4CCCC4)CC3)c2s1] | NT | NT | NT |
| 181 | 385 | 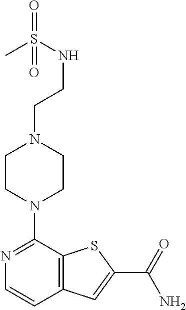 [SMILES: CS(=O)(=O)NCCN1CCN(c2nccc3cc(C(N)=O)sc23)CC1] | NT | NT | NT |
| 182 | 361 | 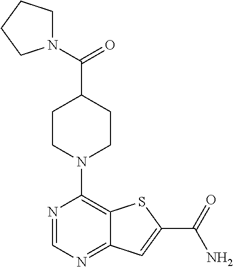 [SMILES: NC(=O)c1cc2ncnc(N3CCC(C(=O)N4CCCC4)CC3)c2s1] | C | B | B |
| 183 | 361 | 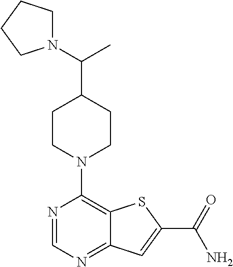 [SMILES: CC(C1CCN(c2ncnc3cc(C(N)=O)sc23)CC1)N1CCCC1] | A | A | A |
| 184 | 419 | 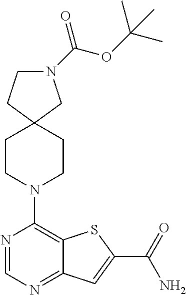 [SMILES: CC(C)(C)OC(=O)N1CCC2(CCN(c3ncnc4cc(C(N)=O)sc34)CC2)C1] | B | A | B |
| 185 | 319 | 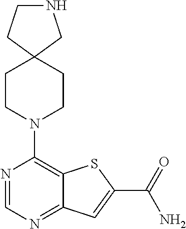 [SMILES: NC(=O)c1cc2ncnc(N3CCC4(CCNC4)CC3)c2s1] | B | B | A |
| 186 | 403 | 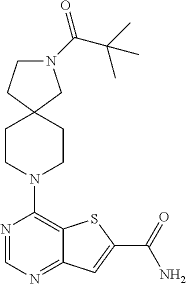 [SMILES: CC(C)(C)C(=O)N1CCC2(CCN(c3ncnc4cc(C(N)=O)sc34)CC2)C1] | A | A | A |

TABLE 8
Additional compounds.

| Compound | [M + H]+ | | Trp | | |
|---|---|---|---|---|---|
| No. | _{(Calc)} | Structure | SIRT1_IC₅₀ | SIRT2_IC₅₀ | SIRT3_IC₅₀ |
| 21 | 264 | 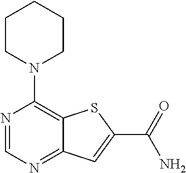 [SMILES: NC(=O)c1cc2ncnc(N3CCCCC3)c2s1] | C | B | B |
| 14 | 215 | 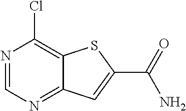 [SMILES: NC(=O)c1cc2ncnc(Cl)c2s1] | C | C | C |
| 22 | 224 | 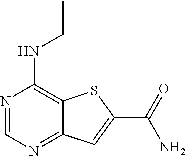 | C | C | C |
| 40 | 392 | 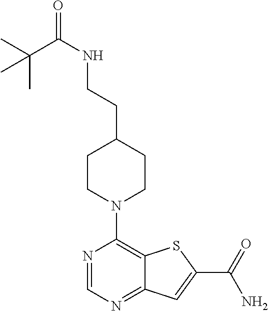 [SMILES: CC(C)(C)C(=O)NCCC1CCN(c2ncnc3cc(C(N)=O)sc23)CC1] | C | C | C |
| 41 | 405 | 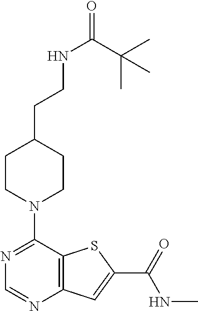 [SMILES: CNC(=O)c1cc2ncnc(N3CCC(CCNC(=O)C(C)(C)C)CC3)c2s1] | C | B | C |
| 42 | 348 | 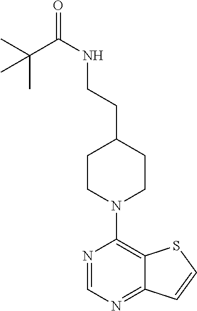 [SMILES: CC(C)(C)C(=O)NCCC1CCN(c2ncnc3ccsc23)CC1] | C | C | C |
| 188 | 264 | 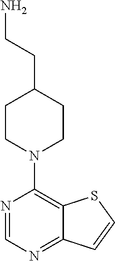 [SMILES: NCCC1CCN(c2ncnc3ccsc23)CC1] | C | B | C |
| 189 | 408 | 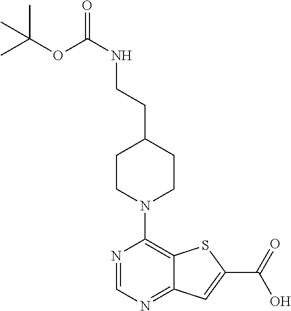 [SMILES: CC(C)(C)OC(=O)NCCC1CCN(c2ncnc3cc(C(=O)O)sc23)CC1] | C | C | C |
| 190 | 439 | 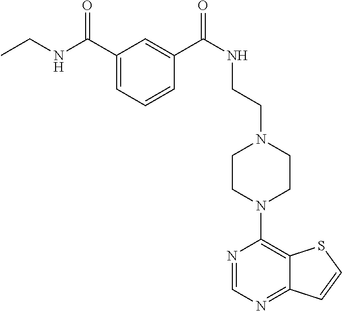 [SMILES: CCNC(=O)c1cccc(C(=O)NCCN2CCN(c3ncnc4ccsc34)CC2)c1] | C | C | C |
| 191 | 454 | 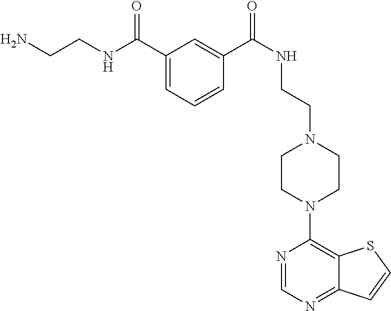 [SMILES: NCCNC(=O)c1cccc(C(=O)NCCN2CCN(c3ncnc4ccsc34)CC2)c1] | C | C | C |
| 192 | 347 | 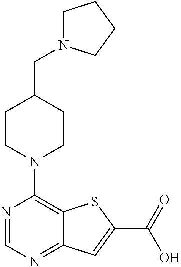 [SMILES: O=C(O)c1cc2ncnc(N3CCC(CN4CCCC4)CC3)c2s1] | C | C | C |
| 193 | 444 | 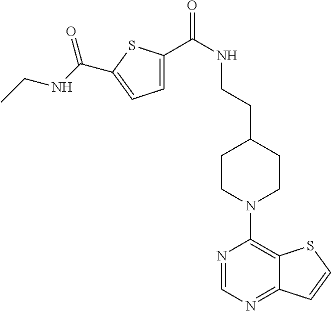 [SMILES: CCNC(=O)c1ccc(C(=O)NCCC2CCN(c3ncnc4ccsc34)CC2)s1] | C | C | C |
| 194 | 515 | 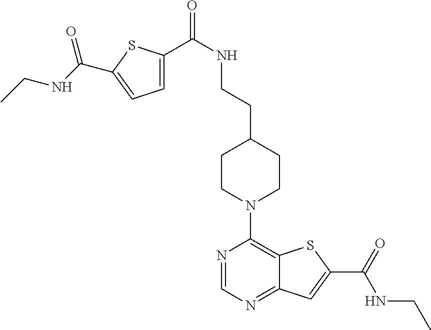 [SMILES: CCNC(=O)c1ccc(C(=O)NCCC2CCN(c3ncnc4cc(C(=O)NCC)sc34)CC2)s1] | B | B | B |

### EQUIVALENTS

The present invention provides among other things sirtuin-modulating compounds and methods of use thereof. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

We claim:

1. A compound of the formula (I): 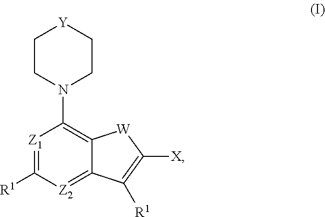
or a salt thereof wherein:
each of Z₁ and Z₂ is independently selected from N and CR¹, wherein:
at least one of Z₁ and Z₂ is N;
each R¹ is independently selected from hydrogen, halo, C₁-C₄ straight chain or branched alkyl, halo substituted C₁-C₄ straight chain or branched alkyl, —O—C₁-C₄ straight chain or branched alkyl, —O-halo-substituted C₁-C₄ straight chain or branched alkyl, C₁-C₄ alkoxy-substituted C₁-C₄ straight chain or branched alkyl, and hydroxy-substituted C₁-C₄ straight chain or branched alkyl;
W is selected from S and O;
X is selected from —C(═O)—NH₂, —S(═O)₂—NH₂, —C(═NH)—NH₂, —C(═O)NHOH, —C(═S)—NH₂, —S(═O)—NH₂ and —SO₃H;
Y is selected from CHR², CR²—(C₁-C₄ straight chain or branched alkyl)-NR³R³, CH—(C₁-C₄ straight chain or branched alkyl)-R², CH—(C₁-C₄ straight chain or branched alkyl)-NR³R³, CH—(C₁-C₄ straight chain or branched alkyl)-NH—C(═O)—R², CH—(C₁-C₄ straight chain or branched alkyl)-NH—C(═S)—R², CH—(C₁-C₄ straight chain or branched alkyl)-C(═O)—NR³R³, N—(C₁-C₄ straight chain or branched alkyl)-NH—C(═O)—R², N—(C₁-C₄ straight chain or branched alkyl)-NH—C(═S)—R², N—(C₁-C₄ straight chain or branched alkyl)-NR³R³, N—(C₁-C₄ straight chain or branched alkyl)-R², and C-linked 5-6 membered saturated heterocycle;
R² is selected from 5- to 6-membered saturated or unsaturated carbocycle or heterocycle, —OH, —O—(C₁-C₄ straight chain or branched alkyl), —C₁-C₄ straight chain or branched alkyl, —S(═O)₂—CH₃, —C(═O)—O—(C₁-C₄ straight chain or branched alkyl), —C(═O)—(C₁-C₄ straight chain or branched alkyl), and when R² is a 5- to 6-membered saturated or unsaturated carbocycle or heterocycle, R² is also optionally substituted with one or more substituents independently selected from halo, —C₁-C₄ straight chain or branched alkyl, —C(═O)—NH—(C₁-C₄ straight chain or branched alkyl), —C(═O)—O—(C₁-C₄ straight chain or branched alkyl), —C(═O)—O—(C₁-C₄ straight chain or branched alkyl), —C(═O)—OH, —O—PO₃H₂ and —C(═O)—NH—(C₁-C₄ straight chain or branched alkyl)-NH₂; and
R³ is independently selected from hydrogen, —C₁-C₄ straight chain or branched alkyl, —C(═O)-(5- to 6-membered saturated carbocycle or heterocycle) and —S(═O)₂—CH₃; or
two R³ bound to the same nitrogen are taken together with the nitrogen atom to form a 5- to 6-membered saturated heterocycle optionally comprising one or two additional heteroatoms selected from N, S, S(═O), S(═O)₂, and O, wherein the heterocycle is optionally substituted at any carbon atom with one or more of —OH, ═O, halo, —C₁-C₄ straight chain or branched alkyl, fluoro-substituted C₁-C₄ straight chain or branched alkyl, hydroxy-substituted C₁-C₄ straight chain or branched alkyl, alkoxy-substituted C₁-C₄ straight chain or branched alkyl, —C(═O)—C₁-C₄ straight chain or branched alkyl, and optionally substituted at any substitutable nitrogen atom with —C₁-C₄ straight chain or branched alkyl, —C(═O)—C₁-C₄ straight chain or branched alkyl,hydroxy-substituted C₁-C₄ straight chain or branched alkyl, alkoxy-substituted C₁-C₄ straight chain or branched alkyl, or halo-substituted C₁-C₄ straight chain or branched alkyl; wherein
when Y is a C-linked 5- to 6-membered heterocycle, it is further optionally substituted at any carbon atom with one or more of —C(═O)—R², —OH, ═O, halo, —C₁-C₄ straight chain or branched alkyl, fluoro-substituted C₁-C₄ straight chain or branched alkyl, hydroxy-substituted C₁-C₄ straight chain or branched alkyl, alkoxy-substituted C₁-C₄ straight chain or branched alkyl, and optionally substituted at any substitutable nitrogen atom with —C₁-C₄ straight chain or branched alkyl, —C(═O)—R², hydroxy-substituted C₁-C₄ straight chain or branched alkyl, alkoxy-substituted C₁-C₄ straight chain or branched alkyl, or halo-substituted C₁-C₄ straight chain or branched alkyl.

2. The compound or salt of claim 1, having the formula:

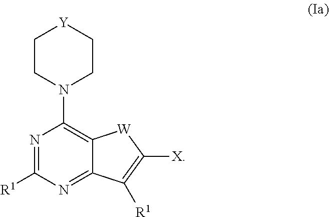

3. The compound or salt of claim 1, selected from:

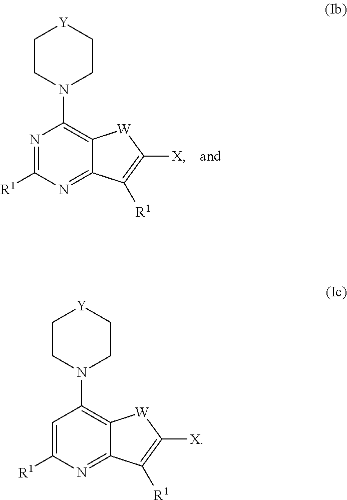

4. The compound or salt of claim 1, wherein W is S.

5. The compound or salt of claim 1, wherein X is —C(═O)—NH₂.

6. The compound or salt of claim 1, wherein Y is selected from CH—(C₁-C₄ straight chain or branched alkyl)-NH—C(═O)—R², CH—(C₁-C₄ straight chain or branched alkyl)-NR³R³, N—(C₁-C₄ straight chain or branched alkyl)-NH—C(═O)—R², N—(C₁-C₄ straight chain or branched alkyl)-NR³R³, CH—(C₁-C₄ straight chain or branched alkyl)-R², and CH—(C₁-C₄ straight chain or branched alkyl)-NH—C(═S)—R².

7. The compound or salt of claim 6, wherein Y is CH—(C₁-C₄ straight chain or branched alkyl)-NH—C(═O)—R², and wherein the compound or salt is selected from any one of:

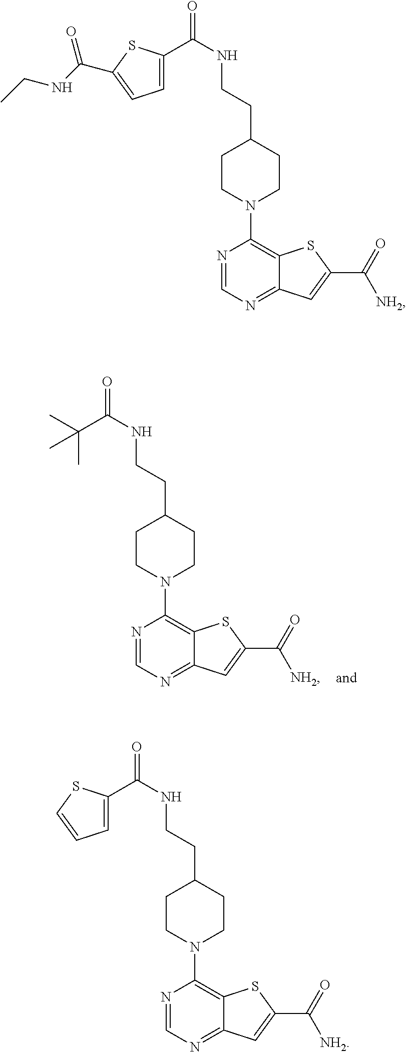 [SMILES: CCNC(=O)c1ccc(C(=O)NCCC2CCN(c3ncnc4cc(C(N)=O)sc34)CC2)s1 ; CC(C)(C)C(=O)NCCC1CCN(c2ncnc3cc(C(N)=O)sc23)CC1 ; "and" ; NC(=O)c1cc2ncnc(N3CCC(CCNC(=O)c4cccs4)CC3)c2s1]

8. The compound or salt of claim 6, wherein Y is CH—(C₁-C₄ straight chain or branched alkyl)-NR³R³, and wherein the compound or salt is selected from any one of:

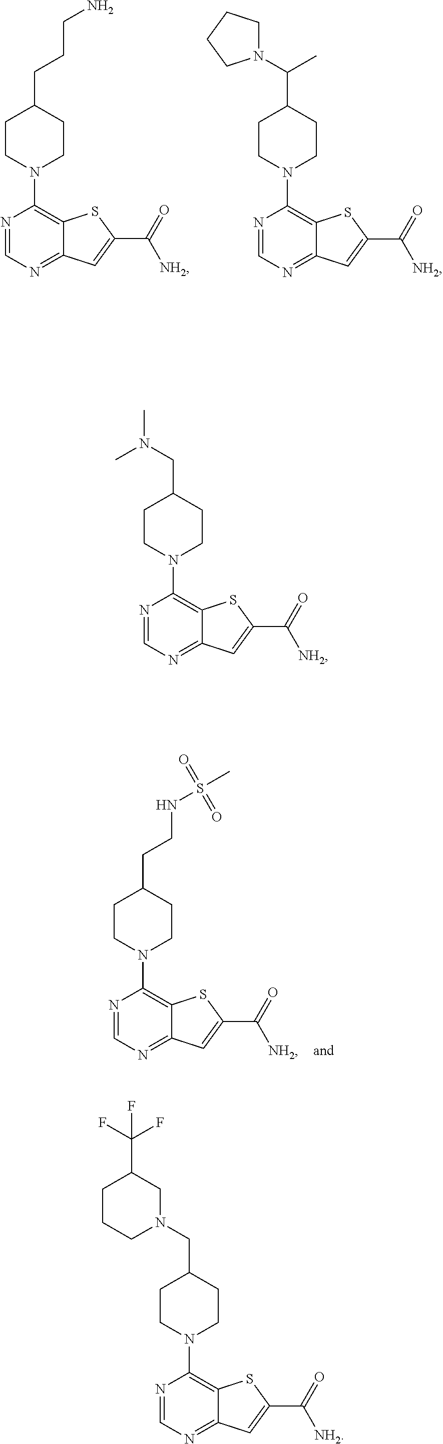 [SMILES: NCCCC1CCN(c2ncnc3cc(C(N)=O)sc23)CC1 ; CC(C1CCN(c2ncnc3cc(C(N)=O)sc23)CC1)N1CCCC1 ; CN(C)CC1CCN(c2ncnc3cc(C(N)=O)sc23)CC1 ; CS(=O)(=O)NCCC1CCN(c2ncnc3cc(C(N)=O)sc23)CC1 ; "and" ; NC(=O)c1cc2ncnc(N3CCC(CN4CCCC(C(F)(F)F)C4)CC3)c2s1]

9. The compound or salt of claim 6, wherein Y is N—(C₁-C₄ straight chain or branched alkyl)-NH—C(═O)—R², and wherein the compound or salt is selected from any one of:

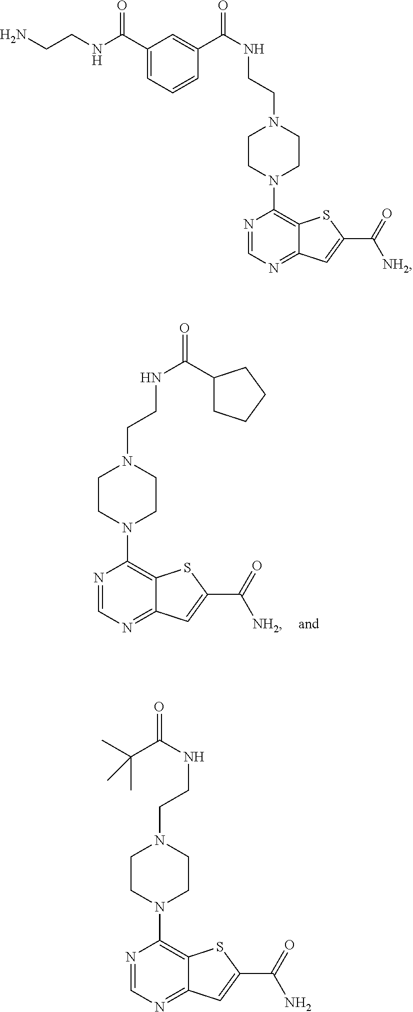 [SMILES: NCCNC(=O)c1cccc(C(=O)NCCN2CCN(c3ncnc4cc(C(N)=O)sc34)CC2)c1 ; NC(=O)c1cc2ncnc(N3CCN(CCNC(=O)C4CCCC4)CC3)c2s1 ; "and" ; CC(C)(C)C(=O)NCCN1CCN(c2ncnc3cc(C(N)=O)sc23)CC1]

10. The compound or salt of claim 6, wherein Y is N—(C₁-C₄ straight chain or branched alkyl)-NR³R³, and wherein the compound or salt is:

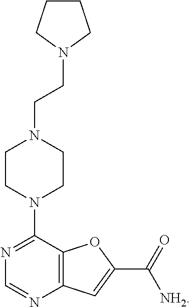 [SMILES: NC(=O)c1cc2ncnc(N3CCN(CCN4CCCC4)CC3)c2o1]

11. The compound or salt of claim 6, wherein Y is CH—(C₁-C₄ straight chain or branched alkyl)-R², and wherein the compound or salt is selected from any one of:

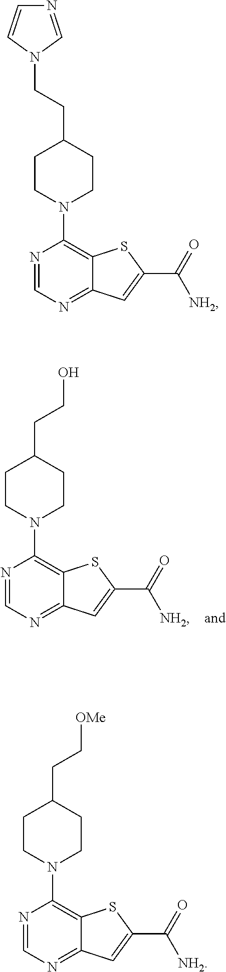 [SMILES: NC(=O)c1cc2ncnc(N3CCC(CCn4ccnc4)CC3)c2s1 ; NC(=O)c1cc2ncnc(N3CCC(CCO)CC3)c2s1 ; "and" ; COCCC1CCN(c2ncnc3cc(C(N)=O)sc23)CC1]

12. The compound or salt of claim 6, wherein Y is CH—(C₁-C₄ straight chain or branched alkyl)-NH—C(═S)—R², and wherein the compound or salt is:

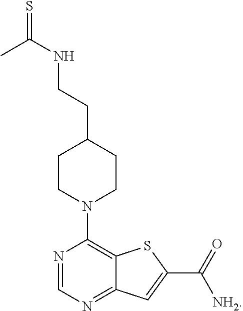 [SMILES: CC(=S)NCCC1CCN(c2ncnc3cc(C(N)=O)sc23)CC1]

13. The compound or salt of claim 1, wherein Y is CHR², and wherein the compound or salt is:

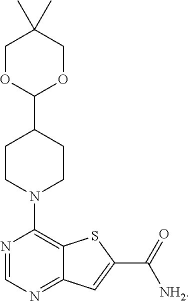 [SMILES: CC1(C)COC(C2CCN(c3ncnc4cc(C(N)=O)sc34)CC2)OC1]

14. The compound or salt of claim 1, wherein Y is a C-linked heterocycle, and wherein the compound or salt is selected from any one of:

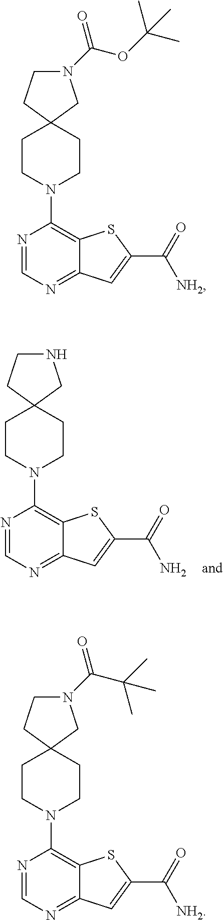 [SMILES: CC(C)(C)OC(=O)N1CCC2(CCN(c3ncnc4cc(C(N)=O)sc34)CC2)C1 ; NC(=O)c1cc2ncnc(N3CCC4(CCNC4)CC3)c2s1 ; "and" ; CC(C)(C)C(=O)N1CCC2(CCN(c3ncnc4cc(C(N)=O)sc34)CC2)C1]

15. The compound or salt of claim 1, wherein R² is selected from a 5- to 6-membered saturated or unsaturated carbocycle or heterocycle, —C₁-C₄ straight chain or branched alkyl, —O—(C₁-C₄ straight chain or branched alkyl), and —OH.

16. The compound or salt of claim 1, wherein R³ is selected from —C₁-C₄ straight chain or branched alkyl and —S(═O)₂—CH₃.

17. The compound or salt of claim 1, wherein two R³ bound to the same nitrogen are taken together with the nitrogen atom to form an optionally substituted 5- to 6-membered saturated heterocycle.

18. A pharmaceutical composition comprising a compound of claim 1 and a pharmaceutically acceptable carrier or diluent.

19. A method for treating a subject suffering from a neurodegenerative disorder or cancer comprising administering to the subject in need thereof a composition of claim 18.

20. A method of detecting sirtuin-dependence in a biological signal comprising comparing the biological signal in the presence of a sirtuin inhibitor compound of claim 1 to the biological signal in the absence of the sirtuin inhibitory compound, wherein an increase or decrease in the biological signal in the presence of the sirtuin inhibitor compound as compared to the biological signal in the absence of the sirtuin inhibitor compound indicates that the biological signal is sirtuin-dependent.
